(19) United States
(12) Patent Application Publication
(10) Pub. No.: US 2025/0382290 A1
(43) Pub. Date: Dec. 18, 2025

(54) SALTS OF SOS1 INHIBITORS

(71) Applicant: MIRATI THERAPEUTICS, INC., San Diego, CA (US)

(72) Inventors: MATTHEW ARNOLD MARX, Chula Vista, CA (US); JOHN MICHAEL KETCHAM, Cary, NC (US); CHRISTOPHER RONALD SMITH, San Diego, CA (US); JOHN DAVID LAWSON, Carlsbad, CA (US); AARON CRAIG BURNS, San Diego, CA (US); XIAOLUN WANG, San Diego, CA (US); SVITLANA KULYK, San Diego, CA (US); ANTHONY IVETAC, San Diego, CA (US)

(73) Assignee: MIRATI THERAPEUTICS, INC., PRINCETON, NJ (US)

(21) Appl. No.: 18/878,118

(22) PCT Filed: Jun. 23, 2023
(86) PCT No.: PCT/US2023/026118

§ 371 Date: Dec. 23, 2024

Related U.S. Application Data
(60) Provisional application No. 63/355,567, filed on Jun. 24, 2022.

(51) Int. Cl. C07D 471/04; A61K 31/5377; C07C 57/15

(52) U.S. Cl. CPC C07D 471/04; A61K 31/5377; C07C 57/15; C07B 2200/13

(57) ABSTRACT

The present invention relates to compounds that inhibit Son of sevenless homolog 1 (SOS1) activity. In particular, the present invention relates to compounds, pharmaceutical compositions and methods of use, such as methods of treating cancer using the compounds and pharmaceutical compositions of the present invention.

### FIELD OF THE INVENTION

The present invention relates to compounds that inhibit Son of sevenless homolog 1 (SOS1) GTP-mediated nucleotide exchange. In particular, the present invention relates to compounds, their salts, pharmaceutical compositions comprising the compounds and methods for use therefor.

### BACKGROUND OF THE INVENTION

The Ras family comprises v-Ki-ras2 Kirsten rat sarcoma viral oncogene homolog (KRAS), neuroblastoma RAS viral oncogene homolog (NRAS), and Harvey murine sarcoma virus oncogene (HRAS) and critically regulates cellular division, growth and function in normal and altered states including cancer (see e.g., Simanshu et al. Cell, 2017. 170(1): p. 17-33; Matikas et al., Crit Rev Oncol Hematol, 2017. 110: p. 1-12). RAS proteins are activated by upstream signals, including receptor tyrosine kinases (RTKs), and transduce signals to several downstream signaling pathways such as the mitogen-activated protein kinase (MAPK)/extracellular signal-regulated kinases (ERK) pathway. Hyperactivation of RAS signaling is frequently observed in cancer as a result of mutations or alterations in RAS genes or other genes in the RAS pathway. The identification of strategies to inhibit RAS and RAS signaling are predicted to be useful for the treatment of cancer and RAS-regulated disease states.

RAS proteins are guanosine triphosphatases (GTPases) that cycle between an inactive, guanosine diphosphate (GDP)-bound state and an active guanosine triphosphate (GTP)-bound state. Son of sevenless homolog 1 (SOS1) is a guanine nucleotide exchange factor (GEF) that mediates the exchange of GDP for GTP, thereby activating RAS proteins. RAS proteins hydrolyze GTP to GDP through their intrinsic GTPase activity which is greatly enhanced by GTPase-activating proteins (GAPs). This regulation through GAPs and GEFs is the mechanism whereby activation and deactivation are tightly regulated under normal conditions. Mutations at several residues in all three RAS proteins are frequently observed in cancer and result in RAS remaining predominantly in the activated state (Sanchez-Vega et al., Cell, 2018. 173: p. 321-337 Li et al., Nature Reviews Cancer, 2018. 18: p. 767-777). Mutations at codon 12 and 13 are the most frequently mutated RAS residues and prevent GAP-stimulated GTP hydrolysis by blocking the interaction of GAP proteins and RAS. Recent biochemical analyses however, demonstrated these mutated proteins still require nucleotide cycling for activation based on their intrinsic GTPase activity and/or partial sensitivity to extrinsic GTPases. As such, mutant RAS proteins are sensitive to inhibition of upstream factors such as SOS1 or SHP2, another upstream signaling molecule required for RAS activation (Hillig, 2019; Patricelli, 2016; Lito, 2016; Nichols, 2018).

The three main RAS-GEF families that have been identified in mammalian cells are SOS, RAS-GRF and RAS-GRP (Rojas, 2011). RAS-GRF and RAS-GRP are expressed in the cells of the central nervous system and hematopoietic cells, respectively, while the SOS family is ubiquitously expressed and is responsible for transducing RTK signaling. The SOS family comprises SOS1 and SOS2 and these proteins share approximately 70% sequence identity. SOS1 appears to be much more active than SOS2 due to the rapid degradation of SOS2. The mouse SOS2 knockout is viable whereas the SOS1 knockout is embryonic lethal. A tamoxifen-inducible SOS1 knockout mouse model was used to interrogate the role of SOS1 and SOS2 in adult mice and demonstrated the SOS1 knockout was viable but the SOS1/2 double knockout was not viable (Baltanas, 2013) suggesting functional redundancy and that selective inhibition of SOS1 may have a sufficient therapeutic index for the treatment of SOS1-RAS activated diseases.

SOS proteins are recruited to phosphorylated RTKs through an interaction with growth factor receptor bound protein 2 (GRB2). Recruitment to the plasma membrane places SOS in close proximity to RAS and enables SOS-mediated RAS activation. SOS proteins bind to RAS through a binding site that promotes nucleotide exchange as well as through an allosteric site that binds GTP-bound RAS-family proteins and increases the function of SOS (Freedman et al., Proc. Natl. Acad. Sci, USA 2006. 103(45): p. 16692-97). Binding to the allosteric site relieves steric occlusion of the RAS substrate binding site and is therefore required for nucleotide exchange. Retention of the active conformation at the catalytic site following interaction with the allosteric site is maintained in isolation due to strengthened interactions of key domains in the activated state. SOS1 mutations are found in Noonan syndrome and several cancers including lung adenocarcinoma, embryonal rhabdomyosarcoma, Sertoli cell testis tumor and granular cell tumors of the skin (see e.g., Denayer, E., et al, Genes Chromosomes Cancer, 2010. 49(3): p. 242-52).

GTPase-activating proteins (GAPs) are proteins that stimulate the low intrinsic GTPase activity of RAS family members and therefore converts active GTP-bound RAS proteins into inactive, GDP-bound RAS proteins (e.g., see Simanshu, D. K., Cell, 2017, Ras Proteins and their Regulators in Human Disease). While activating alterations in the GEF SOS1 occur in cancers, inactivating mutations and loss-of-function alterations in the GAPs neurofibromin 1 (NF-1) or neurofibromin 2 (NF-2) also occur creating a state where SOS1 activity is unopposed and activity downstream of the pathway through RAS proteins is elevated.

Thus, the compounds of the present invention that block the interaction between SOS1 and Ras-family members prevent the recycling of KRas into the active GTP-bound form and, therefore, may provide therapeutic benefit for a wide range of cancers, particularly Ras family member-associated cancers. The compounds of the present invention offer potential therapeutic benefit as inhibitors of SOS1-KRas interaction that may be useful for negatively modulating the activity of KRas through blocking SOS1-KRas interaction in a cell for treating various forms of cancer, including Ras-associated cancer, SOS1-associated cancer and NF1/NF2-associated cancer.

### SUMMARY OF THE INVENTION

There is a need to develop new SOS1 inhibitors that are capable of blocking the interaction between SOS1 and Ras-family members, prevent the recycling of KRas into the active GTP-bound form and, therefore, may provide therapeutic benefit for a wide range of cancers, particularly including Ras-associated cancers, SOS1-associated cancers and NF1/NF2-associated cancers.

In one aspect of the invention, a compound is provided represented by the following formula:

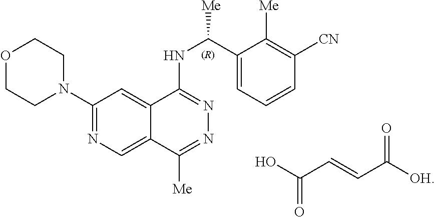

This representation of a fumarate salt is meant to also depict and include salt forms where the fumaric acid moiety is deprotonated and the MRTX-0902 moiety is protonated.

This compound is a fumarate salt of MRTX-0902, the species of Example 12-10 of WO 2021/127429A1, the contents of which are herein incorporated by reference in their entirety.

In another aspect of the invention, the invention provides the maleate salt of MRTX-0902 which has the following structure:

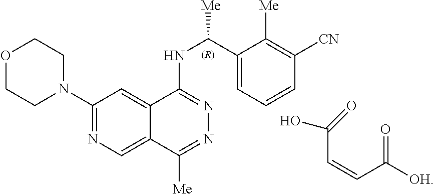

This representation of the maleate salt is meant to also depict and include salt forms where the maleic acid moiety is deprotonated and the MRTX-0902 moiety is protonated.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect, the invention provides methods for inhibiting the activity of a Ras-family member by inhibiting the association between the Ras-family member and SOS1 in a cell, comprising contacting the cell with provided compounds. In one embodiment, the contacting is in vitro. In one embodiment, the contacting is in vivo.

Also provided herein is a method of inhibiting cell proliferation, in vitro or in vivo, the method comprising contacting a cell with an effective amount of provided compounds or a pharmaceutically acceptable salt thereof, or a pharmaceutical composition thereof as defined herein.

Also provided herein are methods for treating cancer in a subject in need thereof, the method comprising (a) determining that cancer is associated with a Ras-family member mutation (e.g., a KRas G12C-associated cancer) (e.g., as determined using a regulatory agency-approved, e.g., FDA-approved, assay or kit); and (b) administering to the patient a therapeutically effective amount of provided compounds, or pharmaceutically acceptable salts or pharmaceutical compositions thereof.

Also provided herein are methods for treating cancer in a subject in need thereof, the method comprising (a) determining that cancer is associated with a SOS1 mutation (e.g., a SOS1-associated cancer) (e.g., as determined using a regulatory agency-approved, e.g., FDA-approved, assay or kit); and (b) administering to the patient a therapeutically effective amount of provided compounds, or pharmaceutically acceptable salts or pharmaceutical compositions thereof.

Also provided herein are methods for treating cancer in a subject in need thereof, the method comprising (a) determining that cancer is associated with a NF-1 or NF-2 loss-of-function mutation (e.g., a NF1/NF2-associated cancer) (e.g., as determined using a regulatory agency-approved, e.g., FDA-approved, assay or kit); and (b) administering to the patient a therapeutically effective amount of provided compounds, or pharmaceutically acceptable salts or pharmaceutical compositions thereof.

Also provided herein is a use of provided compounds, or a pharmaceutically acceptable salt or solvate thereof, as defined herein in the manufacture of a medicament for the inhibition of activity of SOS1.

Also provided herein is the use of provided compounds, or a pharmaceutically acceptable salt or solvate thereof, as defined herein, in the manufacture of a medicament for the treatment of a SOS1-associated disease or disorder.

### DETAILED DESCRIPTION OF THE INVENTION

The present invention relates to SOS1 inhibitors. In particular, the present invention relates to compounds that inhibit SOS1 activity, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly12Cys.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variant p.Gly12Asp.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variant p.Gly12Ser.

As used herein, “KRas G12A” refers to a mutant form of a mammalian KRas protein that contains an amino acid substitution of an alanine for a glycine at amino acid position 12. The assignment of amino acid codon and residue positions for human KRas is based on the amino acid sequence identified by UniProtKB/Swiss-Prot P01116: Variant p.Glyl2Ala.

As used herein, “KRas G13D” refers to a mutant form of a mammalian KRas protein that contains an amino acid substitution of an aspartic acid for a glycine at amino acid position 13. The assignment of amino acid codon and residue positions for human KRas is based on the amino acid sequence identified by UniProtKB/Swiss-Prot P01116: Variant p.Gly13Asp.

As used herein, “KRas G13C” refers to a mutant form of a mammalian KRas protein that contains an amino acid substitution of a cysteine for a glycine at amino acid position 13. The assignment of amino acid codon and residue positions for human KRas is based on the amino acid sequence identified by UniProtKB/Swiss-Prot P01116: Variant p.Glyl3Cys.

As used herein, “KRas Q61L” refers to a mutant form of a mammalian KRas protein that contains an amino acid substitution of a leucine for a glutamine at amino acid position 41. The assignment of amino acid codon and residue positions for human KRas is based on the amino acid sequence identified by UniProtKB/Swiss-Prot P01116: Variant p.Gln61Leu.

As used herein, “KRas A146T” refers to a mutant form of a mammalian KRas protein that contains an amino acid substitution of a threonine for an alanine at amino acid position 146. The assignment of amino acid codon and residue positions for human KRas is based on the amino acid sequence identified by UniProtKB/Swiss-Prot P01116: Variant p.Ala146Thr.

As used herein, “KRas A146V” refers to a mutant form of a mammalian KRas protein that contains an amino acid substitution of a valine for an alanine at amino acid position 146. The assignment of amino acid codon and residue positions for human KRas is based on the amino acid sequence identified by UniProtKB/Swiss-Prot P01116: Variant p.Ala146Val.

As used herein, “KRas A146P” refers to a mutant form of a mammalian KRas protein that contains an amino acid substitution of a proline for an alanine at amino acid position 146. The assignment of amino acid codon and residue positions for human KRas is based on the amino acid sequence identified by UniProtKB/Swiss-Prot P01116: Variant p.Ala146Pro.

As used herein, “HRas G12C” refers to a mutant form of a mammalian HRas protein that contains an amino acid substitution of a cysteine for a glycine at amino acid position 12. The assignment of amino acid codon and residue positions for human HRas is based on the amino acid sequence identified by UniProtKB/Swiss-Prot P01112: Variant p.Glyl2Cys.

As used herein, “HRas G12D” refers to a mutant form of a mammalian HRas protein that contains an amino acid substitution of an aspartic acid for a glycine at amino acid position 12. The assignment of amino acid codon and residue positions for human HRas is based on the amino acid sequence identified by UniProtKB/Swiss-Prot P01112: Variant p.Gly12Asp.

As used herein, “HRas G12S” refers to a mutant form of a mammalian HRas protein that contains an amino acid substitution of a serine for a glycine at amino acid position 12. The assignment of amino acid codon and residue positions for human HRas is based on the amino acid sequence identified by UniProtKB/Swiss-Prot P01112: Variant p.Gly12Ser.

As used herein, “HRas G12A” refers to a mutant form of a mammalian HRas protein that contains an amino acid substitution of an alanine for a glycine at amino acid position 12. The assignment of amino acid codon and residue positions for human KRas is based on the amino acid sequence identified by UniProtKB/Swiss-Prot P01112: Variant p.Glyl2Ala.

As used herein, “HRas G13D” refers to a mutant form of a mammalian HRas protein that contains an amino acid substitution of an aspartic acid for a glycine at amino acid position 13. The assignment of amino acid codon and residue positions for human HRas is based on the amino acid sequence identified by UniProtKB/Swiss-Prot P01112: Variant p.Gly13 Asp.

As used herein, “HRas G13C” refers to a mutant form of a mammalian HRas protein that contains an amino acid substitution of a cysteine for a glycine at amino acid position 13. The assignment of amino acid codon and residue positions for human HRas is based on the amino acid sequence identified by UniProtKB/Swiss-Prot P01112: Variant p.Glyl3Cys.

As used herein, “HRas Q61L” refers to a mutant form of a mammalian HRas protein that contains an amino acid substitution of a leucine for a glutamine at amino acid position 41. The assignment of amino acid codon and residue positions for human HRas is based on the amino acid sequence identified by UniProtKB/Swiss-Prot P01112: Variant p.Gln61Leu.

As used herein, “HRas A146T” refers to a mutant form of a mammalian HRas protein that contains an amino acid substitution of a threonine for an alanine at amino acid position 146. The assignment of amino acid codon and residue positions for human NRas is based on the amino acid sequence identified by UniProtKB/Swiss-Prot P01112: Variant p.Ala146Thr.

As used herein, “HRas A146V” refers to a mutant form of a mammalian HRas protein that contains an amino acid substitution of a valine for an alanine at amino acid position 146. The assignment of amino acid codon and residue positions for human NRas is based on the amino acid sequence identified by UniProtKB/Swiss-Prot P01112: Variant p.Ala146Val.

As used herein, “HRas A146P” refers to a mutant form of a mammalian HRas protein that contains an amino acid substitution of a proline for an alanine at amino acid position 146. The assignment of amino acid codon and residue positions for human NRas is based on the amino acid sequence identified by UniProtKB/Swiss-Prot P01112: Variant p.Ala146Pro.

As used herein, “NRas G12C” refers to a mutant form of a mammalian NRas protein that contains an amino acid substitution of a cysteine for a glycine at amino acid position 12. The assignment of amino acid codon and residue positions for human NRas is based on the amino acid sequence identified by UniProtKB/Swiss-Prot P01111: Variant p.Glyl2Cys.

As used herein, “NRas G12D” refers to a mutant form of a mammalian NRas protein that contains an amino acid substitution of an aspartic acid for a glycine at amino acid position 12. The assignment of amino acid codon and residue positions for human NRas is based on the amino acid sequence identified by UniProtKB/Swiss-Prot P01111: Variant p.Gly12Asp.

As used herein, “NRas G12S” refers to a mutant form of a mammalian NRas protein that contains an amino acid substitution of a serine for a glycine at amino acid position 12. The assignment of amino acid codon and residue positions for human NRas is based on the amino acid sequence identified by UniProtKB/Swiss-Prot P01111: Variant p.Gly12Ser.

As used herein, “NRas G12A” refers to a mutant form of a mammalian NRas protein that contains an amino acid substitution of an alanine for a glycine at amino acid position 12. The assignment of amino acid codon and residue positions for human KRas is based on the amino acid sequence identified by UniProtKB/Swiss-Prot P01111: Variant p.Glyl2Ala.

As used herein, “NRas G13D” refers to a mutant form of a mammalian NRas protein that contains an amino acid substitution of an aspartic acid for a glycine at amino acid position 13. The assignment of amino acid codon and residue positions for human NRas is based on the amino acid sequence identified by UniProtKB/Swiss-Prot P01111: Variant p.Gly13Asp.

As used herein, “HNRas G13C” refers to a mutant form of a mammalian NRas protein that contains an amino acid substitution of a cysteine for a glycine at amino acid position 13. The assignment of amino acid codon and residue positions for human NRas is based on the amino acid sequence identified by UniProtKB/Swiss-Prot P01111: Variant p.Gly13Cys.

As used herein, “HRas Q61L” refers to a mutant form of a mammalian HRas protein that contains an amino acid substitution of a leucine for a glutamine at amino acid position 41. The assignment of amino acid codon and residue positions for human HRas is based on the amino acid sequence identified by UniProtKB/Swiss-Prot P01112: Variant p.Gln61Leu.

As used herein, “NRas A146T” refers to a mutant form of a mammalian NRas protein that contains an amino acid substitution of a threonine for an alanine at amino acid position 146. The assignment of amino acid codon and residue positions for human NRas is based on the amino acid sequence identified by UniProtKB/Swiss-Prot P01111: Variant p.Ala146Thr.

As used herein, “NRas A146V” refers to a mutant form of a mammalian NRas protein that contains an amino acid substitution of a valine for an alanine at amino acid position 146. The assignment of amino acid codon and residue positions for human NRas is based on the amino acid sequence identified by UniProtKB/Swiss-Prot P01111: Variant p.Ala146Val.

As used herein, “NRas A146P” refers to a mutant form of a mammalian NRas protein that contains an amino acid substitution of a proline for an alanine at amino acid position 146. The assignment of amino acid codon and residue positions for human NRas is based on the amino acid sequence identified by UniProtKB/Swiss-Prot P01111: Variant p.Ala146Pro.

As used herein, “a Ras family member” or “Ras family” refers to KRas, HRas, NRas, and activating mutants thereof, including at positions G12, G13, Q61 and A146.

A “Ras family-associated disease or disorder” as used herein refers to diseases or disorders associated with or mediated by or having an activating Ras mutation, such as one at position G12, G13, Q61 or A146. Non-limiting examples of Ras family-associated disease or disorder are a KRas, HRas or NRas G12C-associated cancer, a KRas, HRas or NRas G12D-associated cancer, a KRas, HRas or NRas G12S-associated cancer, a KRas, HRas or NRas G12A-associated cancer, a KRas, HRas or NRas G13D-associated cancer, a KRas, HRas or NRas G13C-associated cancer, a KRas, HRas or NRas Q61X-associated cancer, a KRas, HRas or NRas A146T-associated cancer, a KRas, HRas or NRas A146V-associated cancer or a KRas, HRas or NRas A146P-associated cancer.

As used herein, “SOS1” refers to a mammalian Son of sevenless homolog 1 (SOS1) enzyme.

A “SOS1-associated disease or disorder” as used herein refers to diseases or disorders associated with or mediated by or having an activating SOS1 mutation. Examples of activating SOS1 mutations include SOS1 N233S and SOS1 N233Y mutations.

As used herein, “SOS1 N233S” refers to a mutant form of a mammalian SOS1 protein that contains an amino acid substitution of a serine for a glutamine at amino acid position 233. The assignment of amino acid codon and residue positions for human SOS1 is based on the amino acid sequence identified by UniProtKB/Swiss-Prot Q07889: Variant p.Gln233Ser.

As used herein, “SOS1 N233Y” refers to a mutant form of a mammalian SOS1 protein that contains an amino acid substitution of a tyrosine for a glutamine at amino acid position 233. The assignment of amino acid codon and residue positions for human SOS1 is based on the amino acid sequence identified by UniProtKB/Swiss-Prot Q07889: Variant p.Gln233Tyr.

As used herein, an “SOS1 inhibitor” refers to compounds of the present invention that are represented by the structures as described herein. These compounds are capable of negatively inhibiting all or a portion of the interaction of SOS1 with Ras family mutant or SOS1 activating mutation thereby reducing and/or modulating the nucleotide exchange activity of Ras family member-SOS1 complex.

As used herein, a “NF-1/NF-2-associated disease or disorder” refers to diseases or disorders associated with or mediated by or having a loss-of-function mutation in the neurofibromin (NF-1) gene or neurofibromin 2 (NF-2) gene.

As used herein, a “loss-of-function mutation” refers to any point mutation(s), splice site mutation(s), fusions, nonsense mutations (an amino acid is mutated to a stop codon), in-frame or frame-shifting mutations, including insertions and deletions, and a homozygous deletion of the genes encoding the protein in a target cell or cancer cell that results in a partial or complete loss of the presence, activity and/or function of the encoded protein.

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to—OC1-C6 alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NRK, wherein Rx is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C1-C6)alkyl(C6-C10) aryl, including, without limitation, benzyl, phenethyl, and naphthylmethyl.

A “heterocyclyl” or “heterocyclic” group is a mono- or bicyclic (fused, spiro or bridged) ring structure having from 3 to 12 atoms (3, 4, 5, 6, 7, 8, 9, 10, 11 or 12 atoms), or having from 3 to 12 atoms (3, 4, 5, 6, 7, 8, 9, 10, 11, 12 or 13 atoms), for example 4 to 8 atoms, wherein one or more ring atoms are independently-C(O)—, N, NR⁴, O, S or S(O)₂, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl.

As used herein, “heterocyclyl” refers to a heterocyclyl group covalently linked to another group via a bond.

As used herein, the term “heteroaryl” refers to a group having 5 to 14 ring atoms, preferably 5, 6, 10, 13 or 14 ring atoms; having 6, 10, or 14 π electrons shared in a cyclic array, which may include 1, 2 or 3 rings, and having, in addition to carbon atoms, from one to three heteroatoms that are each independently N, O, or S. “Heteroaryl” also includes fused multicyclic (e.g., bicyclic, tricyclic) ring systems in which one or more of the fused rings is non-aromatic (regardless of which ring is attached), provided that at least one ring is aromatic and at least one ring contains an N, O, or S ring atom.

Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “heteroaralkyl” or “heteroarylalkyl” group comprises a heteroaryl group covalently linked to another group via a bond. Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is an bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12 or G13 mutation (e.g., as determined using a regulatory agency-approved, e.g., FDA-approved, assay or kit). In some embodiments, the subject has a tumor that is positive for a KRas G12C mutation, a KRas G12D mutation, a KRas G12S mutation, a KRas G12A mutation, a KRas G13D mutation or a KRas G13C mutation (e.g., as determined using a regulatory agency-approved assay or kit). The subject can be a subject with a tumor(s) that is positive for a a KRas G12C mutation, a KRas G12D mutation, a KRas G12S mutation, a KRas G12A mutation, a KRas G13D mutation or a KRas G13C mutation (e.g., identified as positive using a regulatory agency-approved, e.g., FDA-approved, assay or kit). The subject can be a subject whose tumors have a KRas G12C mutation, a KRas G12D mutation, a KRas G12S mutation, a KRas G12A mutation, a KRas G13D mutation or a KRas G13C mutation (e.g., where the tumor is identified as such using a regulatory agency-approved, e.g., FDA-approved, kit or assay). In some embodiments, the subject is suspected of having a KRas G12 or G13 gene-associated cancer. In some embodiments, the subject has a clinical record indicating that the subject has a tumor that has a KRas G12C mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

As used herein, “an effective amount” of a compound is an amount that is sufficient to negatively modulate or inhibit the activity of SOS1 enzyme.

As used herein, a “therapeutically effective amount” of a compound is an amount that is sufficient to ameliorate or in some manner reduce a symptom or stop or reverse progression of a condition, or negatively modulate or inhibit the activity of SOS1. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect of the invention, a compound is provided represented by the following formula:

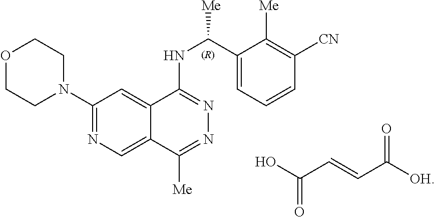

This compound is known as a fumarate salt of MRTX-0902.

In another aspect of the invention, the invention provides a maleate salt of MRTX-0902 which has the following structure:

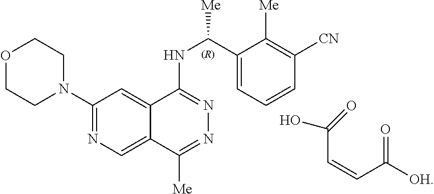

The provided compounds may be formulated into pharmaceutical compositions.

The fumaric salt of MRTX-0902,

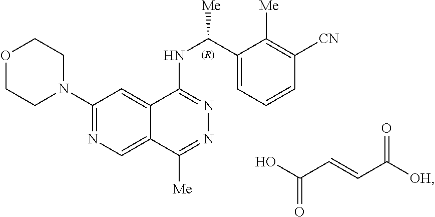

can be prepared as follows:
reacting

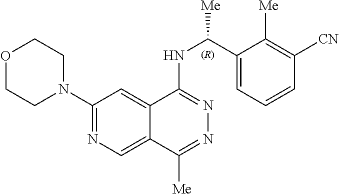

with fumaric acid in the presence of a solvent to produce a compound with the following structure:

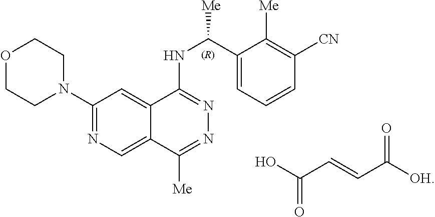

In one embodiment, the solvent is selected from the group consisting of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), N-methylpyrrolidone (NMP), toluene and an alcohol with a formula R—OH, wherein R is alkyl, allyl or aryl. In a preferred embodiment, the solvent is ethanol.

A maleate salt of MRTX-0902,

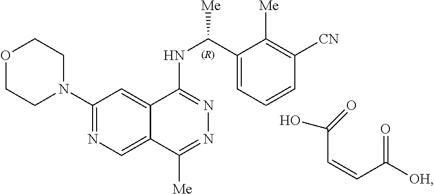

can be prepared using similar techniques (e.g., reacting MRTX-0902 with maleic acid in the presence of a solvent).

### Pharmaceutical Compositions

In another aspect, the invention provides pharmaceutical compositions comprising a SOS1 inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, maleic acid, malic acid, ascorbic acid, benzoic acid, tannic acid, pamoic acid, alginic acid, polyglutamic acid, naphthalenesulfonic acid, naphthalenedisulfonic acid, fumaric acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In yet another aspect, the invention provides for methods for inhibiting SOS1 activity in a cell, comprising contacting the cell in which inhibition of SOS1 activity is desired in vitro with an effective amount of a provided compounds, pharmaceutically acceptable salts thereof or pharmaceutical compositions containing the compound or pharmaceutically acceptable salt thereof.

The compositions and methods provided herein are particularly deemed useful for inhibiting SOS1 activity in a cell. In one embodiment, a cell in which inhibition of SOS1 activity is desired is contacted in vivo with a therapeutically effective amount of a provided compounds to negatively modulate the activity of SOS1. In other embodiments, a therapeutically effective amount of pharmaceutically acceptable salt or pharmaceutical compositions containing the provided compounds may be used. In one embodiment, the cell harbors an activating mutation in a Ras family member, such as KRas, HRas, or NRas. In one embodiment, the cell has aberrant SOS1 activity. In one embodiment, the aberrant SOS1 activity is the result of a SOS1 activating mutation. In one embodiment, the SOS1 activating mutation is a N233S or N233Y mutation. In one embodiment, the cell has aberrant NF-1 or NF-2 activity. In one embodiment, the aberrant NF-1 or NF-2 activity is the result of a NF-1 or NF-2 activating mutation.

By negatively modulating the activity of SOS1, the methods are designed to block the interaction between SOS1 and the Ras family member and increased GTP-loading of RAS proteins thereby decreasing or inhibiting the GTP nucleotide exchange and locking the Ras family member in the GDP-bound, inactive form resulting in the inhibition of downstream Ras-mediated signaling. The cells may be contacted in a single dose or multiple doses in accordance with a particular treatment regimen to affect the desired negative modulation of SOS1.

In another aspect, methods of treating cancer comprising administering to a patient having cancer a therapeutically effective amount of a provided compounds, pharmaceutically acceptable salts thereof or pharmaceutical compositions comprising the compound or pharmaceutically acceptable salts thereof are provided. In one embodiment, the cancer is a Ras family-associated cancer. In one embodiment, the cancer is a SOS-1-associated cancer. In one embodiment, the cancer is a NF-1/NF-2-associated cancer.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is a Ras family-associated cancer, such as a KRas, NRas or HRas-associated cancer. In certain embodiments, the Ras family-associated cancer is non-small cell lung cancer or pancreatic cancer. In one embodiment, the cancer is a SOS1-associated cancer. In certain embodiments, the SOS1-associated cancer is lung adenocarcinoma, embryonal rhabdomyosarcoma, Sertoli cell testis tumor and granular cell tumors of the skin. In one embodiment, the cancer is a NF-1-associated cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### GENERAL REACTION SCHEME, INTERMEDIATES AND EXAMPLES

### General Reaction Schemes

The compounds of the present invention may be prepared using commercially available reagents and intermediates in the synthetic methods and reaction schemes described herein, or may be prepared using other reagents and conventional methods well known to those skilled in the art.

For instance, intermediates for preparing the compounds of the present invention may be prepared according to General Reaction Scheme III:

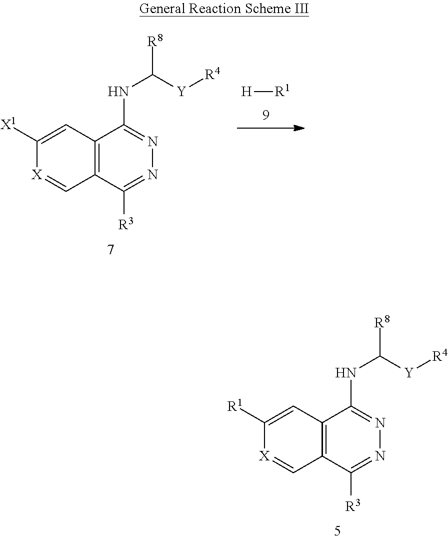

In this General Reaction Scheme III, Compound 7 can either undergo a metal catalyzed reaction or a nucleophilic substitution with a coupling partner, such as an alcohol or amine, H—R¹ 9 in the presence of a suitable base, e.g., cesium carbonate, to form title compound 5.

In this General Reaction Scheme III,
R¹ is hydrogen, hydroxyl, C1-C6 alkyl, alkoxy, —N(R⁶)₂, —NR⁶C(O)R⁶, —C(O)N(R⁶)₂,—SO₂alkyl,—SO₂NR⁶alkyl, cycloalkyl, -Q-heterocyclyl, aryl, or heteroaryl, wherein the cycloalkyl, the heterocyclyl, the aryl, and the heteroaryl are each optionally substituted with one or more R² or L-R²;
each Q is independently a bond, O or NR⁶;
X is N or CR⁷;
each R² is independently C1-C3 alkyl, oxo (i.e., C═O), hydroxy, halogen, cyano, hydroxyalkyl, haloalkyl, alkoxy, —C(O)N(R⁶)₂, —N(R⁶)₂,—SO₂alkyl, —NR⁶C(O)C1-C3 alkyl, —C(O)cycloalkyl, —C(O)C1-C3 alkyl, —C(O)heterocyclyl, aryl, heteroaryl or heterocyclyl, wherein the cycloalkyl, the heterocyclyl, the aryl, the heteroaryl or the heterocyclyl are each optionally substituted with one or more R¹¹;
R³ is hydrogen, C1-C6 alkyl, alkoxy, —N(R¹⁰)₂, -L-N(R¹⁰)₂, cycloalkyl, haloalkyl or heterocyclyl, wherein the C1-C6 alkyl, the cycloalkyl and the heterocyclyl are each optionally substituted with one or more R⁹;
Y is a bond or heteroarylene;
R⁴ is aryl or heteroaryl, each optionally substituted with one or more R⁵;
each R⁵ is independently hydroxy, halogen, cyano, hydroxyalkyl, alkoxy, C1-C3 alkyl, haloalkyl, haloalkyl-OH, —N(R⁶)₂, -L-N(R⁶)₂ or —SO₂alkyl;
L is C1-C3 alkylene;
each R⁶ is independently hydrogen, C1-C3 alkyl, haloalkyl, or cycloalkyl;
R⁷ is hydrogen, cyano, or alkoxy;
R⁸ is C1-C2 alkyl or haloC1-C2 alkyl;
each R⁹ is independently hydroxy, halogen, amino, cyano, alkoxy, or C1-C3 alkyl;
each R¹⁰ is independently hydrogen, C1-C3 alkyl or cycloalkyl;
each R¹¹ is independently C1-C3 alkyl, halogen or haloalkyl; and
R¹² is hydrogen, halogen or C1-C3 alkyl.

The following intermediates may be used to prepare compounds of the present invention.

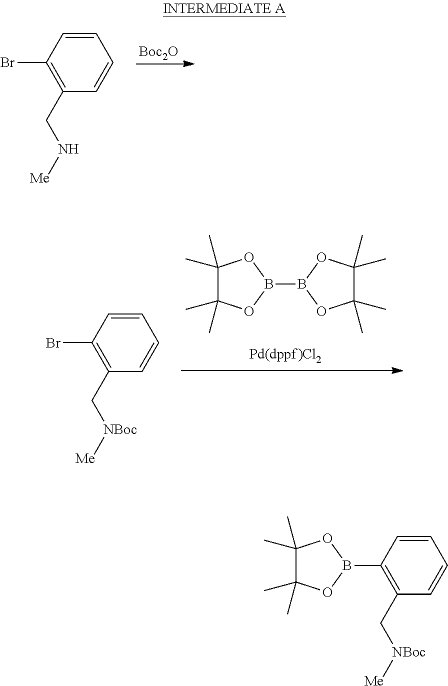

Step A: To a mixture of 1-(2-bromophenyl)-N-methylmethanamine (6.50 g, 32.5 mmol, 1 eq.) in THF (70.0 mL) was added Boc₂O (7.80 g, 35.7 mmol, 8.21 mL, 1.10 eq.) dropwise at 25° C., and the mixture was stirred at 25° C. for 1 hour. The reaction mixture was directly concentrated in vacuo to give a residue. The residue was purified by column chromatography (SiO₂, petroleum ether/ethyl acetate=20/1 to 10/1) to give tert-butyl(2-bromobenzyl)(methyl)carbamate (7.50 g, 25.0 mmol, 76.9% yield) as a colorless oil.

¹H NMR (400 MHZ, CDCl₃) δ 7.55 (br d, J=8.0 Hz, 1H), 7.34-7.28 (m, 1H), 7.22-7.08 (m, 2H), 4.61-4.42 (m, 2H), 2.94-2.78 (m, 3H), 1.60-1.33 (m, 9H).

Step B: A mixture of tert-butyl(2-bromobenzyl)(methyl)carbamate (7.00 g, 23.3 mmol, 1.00 eq.), bis(pinacolato)diboron (8.88 g, 35.0 mmol, 1.50 eq.), Pd (dppf) Cl₂ (1.71 g, 2.33 mmol, 0.10 eq.) and potassium acetate (5.72 g, 58.3 mmol, 2.50 eq.) in dioxane (80.0 mL) was degassed and purged with nitrogen for 3 times, and then the mixture was stirred at 110° C. for 12 hours under a nitrogen atmosphere. The reaction mixture was concentrated under reduced pressure to give a residue, and the residue was purified by column chromatography (SiO₂, petroleum ether/ethyl acetate=1/0 to 10/1) to give tert-butyl methyl(2-(4,4,5,5-tetramethyl-1,3,2-dioxaborolan-2-yl)benzyl)carbamate (8.00 g, 23.0 mmol, 98.8% yield) as a colorless oil.

¹H NMR (400 MHZ, CDCl₃) δ 7.82 (br d, J=7.2 Hz, 1H), 7.48-7.37 (m, 1H), 7.27-7.21 (m, 2H), 4.85-4.63 (m, 2H), 2.92-2.73 (m 3H), 1.54-1.41 (m, 9H), 1.35 (s, 12H).

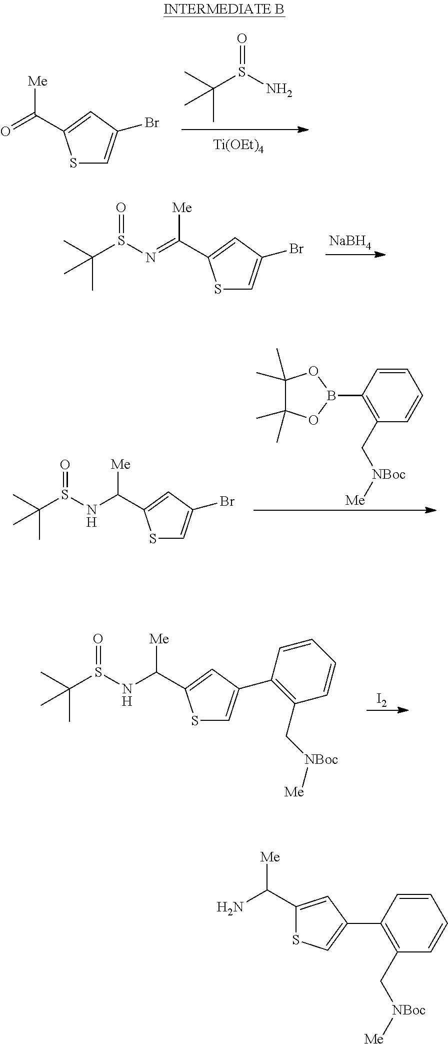

Step A: To a solution of 1-(4-bromothiophen-2-yl)ethan-1-one (4.00 g, 19.5 mmol, 1.10 eq.) and 2-methylpropane-2-sulfinamide (2.15 g, 17.7 mmol, 1.00 eq.) in THF (56.0 mL) was added Ti(OEt)₄ (8.09 g, 35.5 mmol, 7.35 mL, 2.00 eq.). The mixture was stirred at 70° C. for 2 hours. The mixture was poured into water (15.0 mL) and stirred for 5 minutes. The suspension was filtered, and filtrate was concentrated in vacuo to give a residue. The residue was washed with petroleum ether/ethyl acetate=5/1 (10 mL), filtered, and filter cake was collected and dried in vacuo to give N-(1-(4-bromothiophen-2-yl)ethylidene)-2-methylpropane-2-sulfinamide (3.00 g, 9.73 mmol, 54.9% yield) as a yellow solid.

¹H NMR (400 MHZ, CDCl₃) δ 7.43 (d, J=1.2 Hz, 1H), 7.41 (d, J=1.2 Hz, 1H), 2.72 (s, 3H), 1.30 (s, 9H).

Step B: To a solution of N-(1-(4-bromothiophen-2-yl)ethylidene)-2-methylpropane-2-sulfinamide (3.70 g, 12.0 mmol, 1.00 eq.) in THF (40.0 mL) was added sodium borohydride (1.36 g, 36.0 mmol, 3.00 eq.) at 0° C. The reaction mixture was warmed slowly to 25° C. and stirred for 2 hours. The mixture was poured into ice-water (15.0 mL) and stirred for 5 minutes at 0° C. The aqueous phase was extracted with ethyl acetate (30.0 mL×3). The combined organic phases were washed with brine (30.0 mL×3), dried over anhydrous sodium sulfate, filtered, and concentrated in vacuo to give N-(1-(4-bromothiophen-2-yl)ethyl)-2-methylpropane-2-sulfinamide (3.60 g, 9.51 mmol, 79.3% yield, 82.0% purity) as yellow oil.

¹H NMR (400 MHZ, CDCl₃) δ 7.15 (s, 1H), 6.98-6.96 (s, 1H), 4.81-4.75 (m, 1H), 3.55 (br d, J=3.6 Hz, 1H), 1.59 (d, J=6.4 Hz, 3H), 1.24 (s, 9H).

Step C: To a solution of N-(1-(4-bromothiophen-2-yl)ethyl)-2-methylpropane-2-sulfinamide (3.00 g, 9.67 mmol, 1.00 eq.) and tert-butyl methyl(2-(4,4,5,5-tetramethyl-1,3,2-dioxaborolan-2-yl)benzyl)carbamate (5.04 g, 14.5 mmol, 1.50 eq.) in dioxane (35.0 mL) and water (8.00 mL) was added Pd(PPh₃)₄ (1.12 g, 967 μmol, 0.10 eq.) and cesium carbonate (9.45 g, 29.01 mmol, 3.00 eq.) under a nitrogen atmosphere. The mixture was stirred at 110° C. for 2 hours under a nitrogen atmosphere. The mixture was filtered, and the filtrate was concentrated in vacuo to give a residue. The residue was purified by column chromatography (SiO₂, petroleum ether/ethyl acetate=10/1 to 1/1) to give tert-butyl(2-(5-(1-((tert-butylsulfinyl)amino)ethyl)thiophen-3-yl)benzyl)(methyl)carbamate (1.40 g, 3.11 mmol, 32.1% yield) as yellow oil. LCMS [M+1]: 451.2.

Step D: To a solution of tert-butyl(2-(5-(1-((tert-butylsulfinyl)amino)ethyl)thiophen-3-yl)benzyl)(methyl)carbamate (1.40 g, 4.88 mmol, 1.00 eq.) in THF (15.0 mL) and water (5.00 mL) was added iodine (232 mg, 1.46 mmol, 295 μL, 0.30 eq.). The mixture was stirred at 50° C. for 30 minutes. The residue was poured into saturated sodium sulfite aqueous solution (30.0 mL) and stirred for 5 minutes. The aqueous phase was extracted with ethyl acetate (15.0 mL×2). The combined organic phases were washed with brine (30.0 mL×2), dried over anhydrous sodium sulfate, filtered and concentrated in vacuo to give tert-butyl(2-(5-(1-aminoethyl)thiophen-3-yl)benzyl)(methyl)carbamate (1.20 g, crude) as yellow oil.

¹H NMR (400 MHZ, CDCl₃) δ 7.36-7.28 (m, 3H), 7.26-7.22 (m, 1H), 7.01 (s, 1H), 6.91 (br s, 1H), 4.49 (br d, J=19.2 Hz, 2H), 4.40 (q, J=6.4 Hz, 1H), 2.72 (br d, J=19.2 Hz, 3H), 1.53 (d, J=6.4 Hz, 3H), 1.51-1.40 (m, 9H).

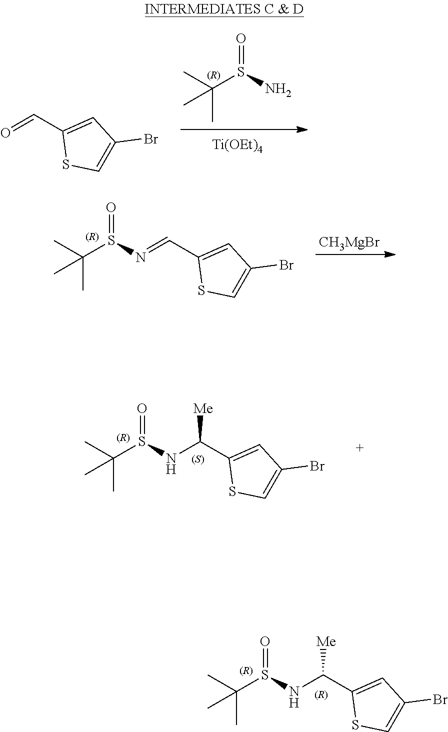

Step A: To a solution of 4-bromothiophene-2-carbaldehyde (20.0 g, 104 mmol, 1.00 eq.) and (R)-2-methylpropane-2-sulfinamide (12.1 g, 99.5 mmol, 0.95 eq.) in THF (200 mL) was added titanium (IV) ethoxide (47.8 g, 209 mmol, 43.4 mL, 2.00 eq.). The reaction mixture was stirred at 25° C. for 1 hour. The mixture was then poured into water (20.0 mL) and stirred for 5 minutes to give a suspension. The suspension was filtered and the filtered liquor was concentrated in vacuo to give (R,E)-N-((4-bromothiophen-2-yl)methylene)-2-methylpropane-2-sulfinamide (20.0 g, crude) as yellow oil. LCMS [M+1]: 295.8.

Step B: To a solution of (R,E)-N-((4-bromothiophen-2-yl)methylene)-2-methylpropane-2-sulfinamide (600 mg, 2.04 mmol, 1.00 eq.) in THF (200 mL) was added methyl magnesium bromide (3.00 M, 2.04 mL, 3.00 eq.) dropwise at 0° C. Then the reaction mixture was stirred at 25° C. for 1 hour. Saturated ammonium chloride aqueous solution (3.00 mL) was added to the reaction mixture and stirred for 5 minutes. The aqueous phase was extracted with ethyl acetate (3.00 mL×2), and the combined organic phases were washed with brine (3.00 mL×2), dried over anhydrous sodium sulfate, filtered and concentrated in vacuo to give a residue. The residue was purified by prep-TLC (SiO₂, petroleum ether/ethyl acetate=1/1) to give (R)—N—((S)-1-(4-bromothiophen-2-yl)ethyl)-2-methylpropane-2-sulfinamide (first eluting, Intermediate C) (120 mg, 19.0% yield) as yellow oil and (R)—N—((R)-1-(4-bromothiophen-2-yl)ethyl)-2-methylpropane-2-sulfinamide (2^{nd} eluting, Intermediate D) (150 mg, 483 μmol, 23.7% yield) as yellow oil.

Intermediate C: ¹H NMR (400 MHZ, CDCl₃) δ 7.15 (d, J=1.6 Hz, 1H), 6.97 (s, 1H), 4.81-4.75 (m, 1H), 3.51 (br d, J=3.2 Hz, 1H), 1.59 (d, J=6.8 Hz, 3H), 1.24 (s, 9H).

Intermediate D: ¹H NMR (400 MHZ, CDCl₃) δ 7.14 (d, J=1.6 Hz, 1H), 6.89 (s, 1H), 4.81-4.74 (m, 1H), 3.39 (br d, J=5.6 Hz, 1H), 1.65 (d, J=6.8 Hz, 3H), 1.25 (s, 9H).

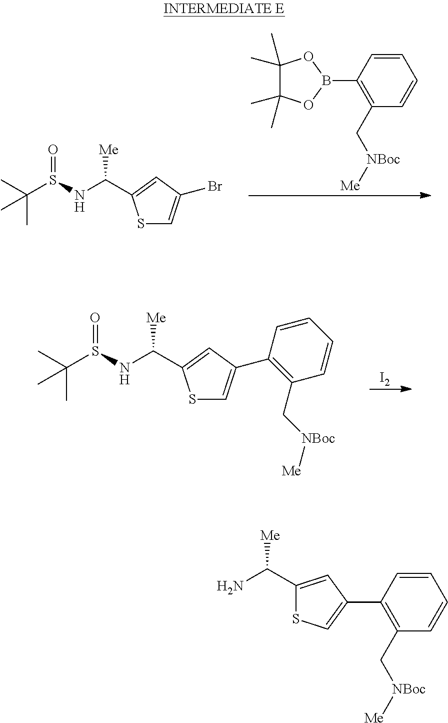

Step A: To a solution of (R)—N—((R)-1-(4-bromothiophen-2-yl)ethyl)-2-methylpropane-2-sulfinamide (150 mg, 483 μmol, 1.00 eq.) and tert-butyl methyl(2-(4,4,5,5-tetramethyl-1,3,2-dioxaborolan-2-yl)benzyl)carbamate (168 mg, 483 μmol, 1.00 eq.) in dioxane (1.00 mL) and water (0.20 mL) was added Pd(PPh₃)₄ (55.9 mg, 48.3 μmol, 0.10 eq) and cesium carbonate (473 mg, 1.45 mmol, 3.00 eq.) under a nitrogen atmosphere. The reaction mixture was stirred at 110° C. for 2 hours under a nitrogen atmosphere, then to 25° C. and concentrated in vacuo to give a residue. The residue was purified by prep-TLC (SiO₂, petroleum ether/ethyl acetate=1/1) to give tert-butyl(2-(5-((R)-1-(((R)-tert-butylsulfinyl)amino)ethyl)thiophen-3-yl)benzyl)(methyl)carbamate (120 mg, 266 μmol, 55.1% yield) as a white solid. LCMS [M+1]=451.1.

¹H NMR (400 MHZ, CDCl₃) δ 7.37-7.29 (m, 3H), 7.25 (s, 1H), 7.06 (s, 1H), 6.95 (br s, 1H), 4.88-4.81 (m, 1H), 4.48 (br d, J=16.0 Hz, 2H), 3.44 (br d, J=6.0 Hz, 1H), 2.73 (br d, J=12.8 Hz, 3H), 1.71 (d, J=6.4 Hz, 3H), 1.27 (s, 9H), 1.25 (s, 9H).

Step B: To a solution of tert-butyl(2-(5-((R)-1-(((R)-tert-butylsulfinyl)amino)ethyl)thiophen-3-yl)benzyl)(methyl)carbamate (120 mg, 266 μmol, 1.00 eq.) in THF (1.00 mL) and water (0.20 mL) was added iodine (20.3 mg, 79.9 μmol, 16.1 μL, 0.30 eq.), and the reaction mixture was stirred at 50° C. for 1 hour. The reaction mixture was then cooled to 25° C., poured into saturated sodium sulfite aqueous solution (2.00 mL) and stirred for 5 minutes. The aqueous phase was extracted with ethyl acetate (3.00 mL×3), and the combined organic phases were washed with brine (3.00 mL×3), dried over anhydrous sodium sulfate, filtered, and concentrated in vacuo to give a residue. The residue was purified by prep-HPLC (column: Phenomenex Gemini-NX C18 75×30 mm×3 um; mobile phase: [water (0.1% TFA)-ACN]; B %: 28%-38%) to give tert-butyl (R)-(2-(5-(1-aminoethyl)thiophen-3-yl)benzyl)(methyl)carbamate (40.0 mg, 113 μmol, 42.3% yield, 97.5% purity) as white oil.

¹H NMR (400 MHZ, CD₃OD) δ 7.41-7.23 (m, 6H), 4.84-4.79 (m, 1H), 4.48 (s, 2H), 2.73 (s, 3H), 1.76 (d, J=6.8 Hz, 3H), 1.51-1.36 (m, 9H).

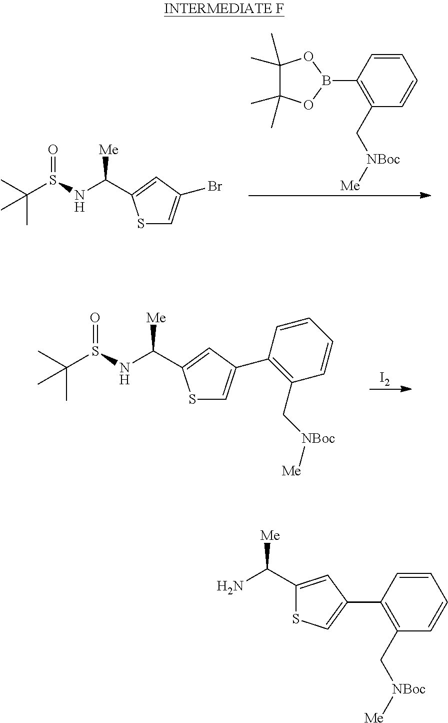

Step A: To a solution of (R)—N—((S)-1-(4-bromothiophen-2-yl)ethyl)-2-methylpropane-2-sulfinamide (100 mg, 322 μmol, 1.00 eq.) and tert-butyl methyl(2-(4,4,5,5-tetramethyl-1,3,2-dioxaborolan-2-yl)benzyl)carbamate (112 mg, 322 μmol, 1.00 eq.) in dioxane (1.00 mL) and water (0.20 mL) was added Pd(PPh₃)₄ (37.2 mg, 32.2 μmol, 0.10 eq.) and cesium carbonate (315 mg, 967 ummol, 3.00 eq.) under a nitrogen atmosphere. The reaction mixture was stirred at 110° C. for 2 hours, then cooled to 25° C. and concentrated in vacuo to give a residue. The residue was purified by prep-TLC (SiO₂, petroleum ether/ethyl acetate=1/1) to give tert-butyl(2-(5-((S)-1-(((R)-tert-butylsulfinyl)amino)ethyl)thiophen-3-yl)benzyl)(methyl)carbamate (100 mg, 266 μmol, 68.9% yield) as yellow oil. LCMS [M+1]=451.1.

¹H NMR (400 MHZ, CDCl₃) δ 7.37-7.28 (m, 3H), 7.26-7.22 (m, 1H), 7.07 (d, J=1.2 Hz, 1H), 7.03 (br s, 1H), 4.90-4.83 (m, 1H), 4.55-4.41 (m, 2H), 3.71-3.55 (m, 1H), 2.80-2.65 (m, 3H), 1.64 (d, J=6.8 Hz, 3H), 1.52-1.41 (m, 9H), 1.26 (s, 9H).

Step B: To a solution of tert-butyl(2-(5-((S)-1-(((R)-tert-butylsulfinyl)amino)ethyl)thiophen-3-yl)benzyl)(methyl)carbamate (100 mg, 266 μmol, 1.00 eq.) in THF (1.00 mL) and water (0.20 mL) was added iodine (16.9 mg, 66.6 μmol, 13.4 μL, 0.30 eq.). The reaction mixture was stirred at 50° C. for 1 hour, thens cooled to 25° C. and poured into saturated aqueous sodium sulfite (2.00 mL) solution and stirred for 5 minutes. The aqueous phase was extracted with ethyl acetate (3.00 mL×3), and the combined organic phases were washed with brine (3.00 mL×3), dried over anhydrous sodium sulfate, filtered, and concentrated in vacuo to give a residue. The residue was purified by prep-HPLC (column: Phenomenex Luna C18 150×25 mm×10 um; mobile phase: [water (0.1% TFA)-ACN]; B %: 24%-54%) to give tert-butyl (S)-(2-(5-(1-aminoethyl)thiophen-3-yl)benzyl)(methyl)carbamate (45.0 mg, 97.7 μmol, 44.0% yield, TFA salt) as white oil. LCMS [M+1]=347.2.

¹H NMR (400 MHZ, CD₃OD) δ 7.40 (d, J=1.2 Hz, 1H), 7.38-7.22 (m, 5H), 4.82-4.80 (br s, 1H), 4.48 (s, 2H), 2.73 (s, 3H), 1.75 (d, J=6.8 Hz, 3H), 1.50-1.35 (m, 9H).

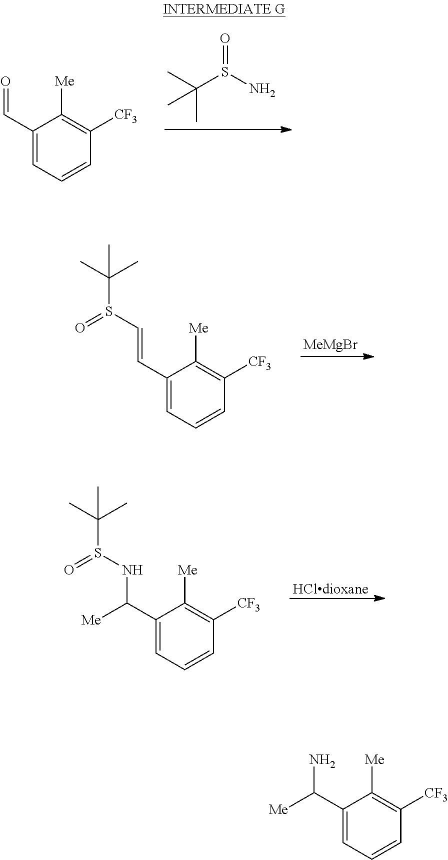

Step A: To a solution of 2-methyl-3-(trifluoromethyl)benzaldehyde (300 mg, 1.59 mmol, 1.00 eq.) and 2-methylpropane-2-sulfinamide (213 mg, 1.75 mmol, 1.10 eq.) in THF (5.00 mL) was added titanium (IV) ethoxide (727 mg, 3.19 mmol, 661 μL, 2.00 eq). The reaction mixture was stirred at 25° C. for 12 hours. The reaction mixture was poured into water (2.00 mL) and stirred for 5 minutes to give a suspension. The suspension was filtered and concentrated in vacuo to give 2-methyl-N-(2-methyl-3-(trifluoromethyl)benzylidene) propane-2-sulfinamide (360 mg, 1.24 mmol, 77.5% yield) as a white solid.

¹H NMR (400 MHZ, CDCl₃) δ=8.98 (s, 1H), 8.13 (d, J=7.6 Hz, 1H), 7.78 (d, J=7.6 Hz, 1H), 7.40 (t, J=7.6 Hz, 1H), 2.70 (d, J=0.8 Hz, 3H), 1.29 (s, 9H).

Step B: To a solution of 2-methyl-N-(2-methyl-3-(trifluoromethyl)benzylidene) propane-2-sulfinamide (185 mg, 635 μmol, 1.00 eq.) in THF (5.00 mL) was added dropwise methyl magnesium bromide (227 mg, 3.00 M, 635 μL, 3.00 eq.) at 0° C. under a nitrogen atmosphere. The reaction mixture was stirred at 25° C. for 3 hours then treated with saturated ammonium chloride solution (10.0 mL) slowly. The organic layer and aqueous phase were separated, and the aqueous phase was extracted with ethyl acetate (5.00 mL×3). The combined organic layers were washed with brine (10.0 mL), dried over anhydrous sodium sulfate, filtered, and concentrated to give a residue. The residue was purified by column chromatography (SiO₂, petroleum ether/ethyl acetate=10/1 to 1/1) to give 2-methyl-N-(1-(2-methyl-3-(trifluoromethyl)phenyl)ethyl)propane-2-sulfinamide (150 mg, 488.0 μmol, 76.8% yield) as a yellow solid.

¹H NMR (400 MHZ, CDCl₃) δ=7.65-7.54 (m, 4H), 7.35-7.28 (m, 2H), 5.00-4.87 (m, 2H), 2.49 (s, 6H), 1.54-1.50 (m, 6H), 1.26-1.24 (m, 9H), 1.22 (s, 9H).

Step C: To a solution of 2-methyl-N-(1-(2-methyl-3-(trifluoromethyl)phenyl)ethyl)propane-2-sulfinamide (150 mg, 488.0 μmol, 1.00 eq.) in HCl (4.0 M in dioxane, 1.00 mL) was stirred at 25° C. for 1 hour. The reaction mixture was filtered and filter cake was concentrated in vacuo to give 1-(2-methyl-3-(trifluoromethyl)phenyl)ethan-1-amine (45.0 mg, 38.5% yield) as a red solid. LCMS [M+1]=204.3.

¹H NMR (400 MHZ, CD₃OD) δ=7.78-7.65 (m, 2H), 7.56-7.48 (m, 1H), 4.93-4.89 (m, 1H), 2.52 (d, J=0.8 Hz, 3H), 1.63 (d, J=6.8 Hz, 3H).

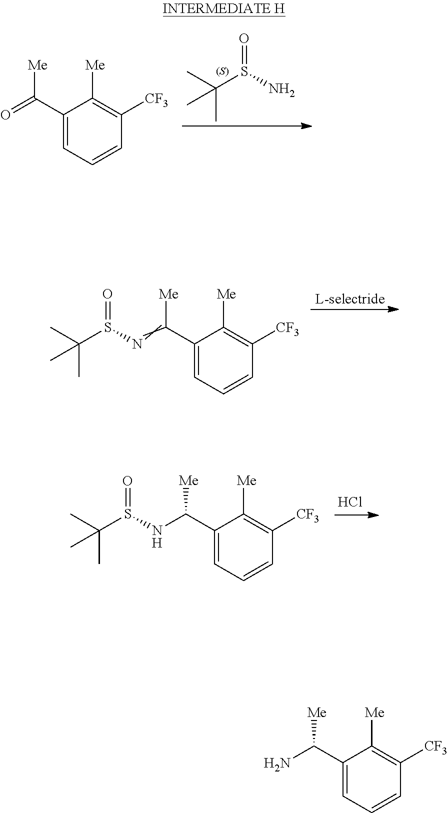

Step A: To a solution of 1-(2-methyl-3-(trifluoromethyl)phenyl)ethan-1-one (8.00 g, 39.6 mmol, 1.00 eq.) and (S)-2-methylpropane-2-sulfinamide (5.28 g, 43.5 mmol, 1.10 eq.) in THF (80.0 mL) was added titanium (IV) ethoxide (18.1 g, 79.1 mmol, 16.4 mL, 2.00 eq.). The reaction mixture was stirred at 70° C. for 2 hours. The reaction mixture was cooled at 25° C. and poured into ice-water (w/w=1/1) (80.0 mL) and stirred for 15 minutes to give a suspension. The suspension was filtered, the filtrate was extracted with ethyl acetate (50.0 mL×3). The combined organic phases were washed with brine (30.0 mL×3), dried over anhydrous sodium sulfate, filtered, and concentrated in vacuo to give a residue. The residue was purified by column chromatography (SiO₂, petroleum ether/ethyl acetate=20/1 to 3/1) to give (S)-2-methyl-N-(1-(2-methyl-3-(trifluoromethyl)phenyl)ethylidene) propane-2-sulfinamide (8.00 g, 26.2 mmol, 66.2% yield) as yellow oil. LCMS [M+1]: 306.2.

¹H NMR (400 MHZ, CD₃OD) δ 7.74 (br t, J=7.2 Hz, 2H), 7.57-7.51 (m, 1H), 7.46 (br t, J=7.6 Hz, 2H), 7.43-7.30 (m, 1H), 2.72 (s, 3H), 2.54 (J=6.8 Hz, 3H), 2.48 (s, 3H), 2.40 (br d, J=16.0 Hz, 3H), 1.31 (s, 9H), 1.24 (br d, J=12.4 Hz, 9H).

Step B: To a solution of S)-2-methyl-N-(1-(2-methyl-3-(trifluoromethyl)phenyl)ethylidene) propane-2-sulfinamide (8.00 g, 26.2 mmol, 1.00 eq.) in THF (80.0 mL) was added L-selectride (7.47 g, 39.3 mmol, 8.59 mL, 1.50 eq.) dropwise at −78° C. The reaction mixture was stirred at −78° C. for 2 hours. Water was added dropwise to the reaction mixture (10.0 mL) at 0° C. and the resulting mixture was stirred for 5 minutes. The aqueous phase was extracted with ethyl acetate (30.0 mL×3). The combined organic phases were washed with brine (30.0 mL×2), dried over anhydrous sodium sulfate, filtered, and concentrated in vacuo to give a residue. The residue was purified by column chromatography (SiO₂, petroleum ether/ethyl acetate=20/1 to 3/1) to give (S)-2-methyl-N—((R)-1-(2-methyl-3-(trifluoromethyl)phenyl)ethyl)propane-2-sulfinamide (3.50 g, 11.4 mmol, 43.5% yield) as yellow oil. LCMS [M+1]: 308.0.

¹H NMR (400 MHZ, CD₃OD) δ=7.70 (d, J=8.0 Hz, 1H), 7.57 (d, J=7.6 Hz, 1H), 7.39-7.33 (m, 1H), 4.94-4.88 (m, 1H), 2.48 (d, J=1.2 Hz, 3H), 1.54 (d, J=6.4 Hz, 3H), 1.20 (s, 9H).

Step C: A solution of S)-2-methyl-N—((R)-1-(2-methyl-3-(trifluoromethyl)phenyl)ethyl)propane-2-sulfinamide (1.30 g, 4.23 mmol, 1.00 eq.) in HCl (4M in dioxane, 15.0 mL) was stirred at 25° C. for 30 minutes. The reaction mixture was filtered and filter cake dried in vacuo to give (R)-1-(2-methyl-3-(trifluoromethyl)phenyl)ethan-1-amine (700 mg, 2.89 mmol, 68.4% yield, 99.1% purity, hydrochloride) as a white solid. LCMS [M+H]: 204.0.

¹H NMR (400 MHZ, CD₃OD) δ=7.73 (t, J=7.6 Hz, 2H), 7.54-7.49 (m, 1H), 4.92-4.88 (m, 1H), 2.52 (d, J=0.8 Hz, 3H), 1.62 (d, J=6.8 Hz, 3H).

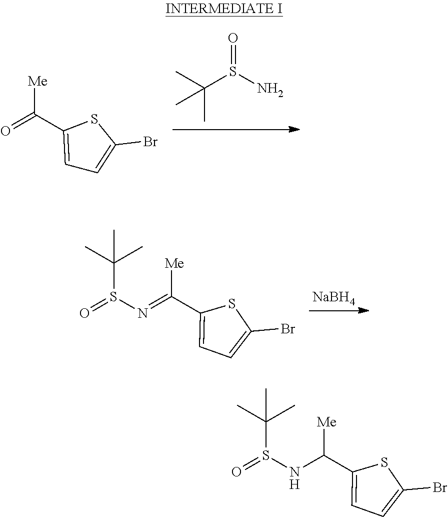

Step A: To a solution of 1-(5-bromothiophen-2-yl)ethan-1-one (11.0 g, 53.6 mmol, 1.00 eq.) in THF (120 mL) was added 2-methylpropane-2-sulfinamide (8.45 g, 69.7 mmol, 1.30 eq.) and titanium (IV) ethoxide (24.5 g, 107 mmol, 22.3 mL, 2.00 eq.), the reaction mixture was stirred at 75° C. for 12 hours under a nitrogen atmosphere. The reaction mixture was cooled to 25° C. and concentrated in vacuo to give a residue, the residue was diluted with water (200 mL) and ethyl acetate (200 mL), filtered, and the filtrate was extracted with ethyl acetate (100 mL×3). The combined organic layers were washed with brine (300 mL), dried over anhydrous sodium sulfate, filtered, and concentrated under reduced pressured to give N-(1-(5-bromothiophen-2-yl)ethylidene)-2-methylpropane-2-sulfinamide (16.0 g, crude) as a yellow solid. LCMS [M+1]: 308.0.

Step B: To a solution of N-(1-(5-bromothiophen-2-yl)ethylidene)-2-methylpropane-2-sulfinamide (16.0 g, 51.9 mmol, 1.00 eq.) in THF (150 mL) was added sodium borohydride (3.93 g, 104 mmol, 2.00 eq.) at 0° C., the reaction mixture was stirred at 20° C. for 1 hour. Saturated sodium bicarbonate aqueous solution (20.0 mL) was added to the reaction mixture dropwise, then the mixture was diluted with water (200 mL) and extracted with ethyl acetate (100 mL×3). The combined organic layers were dried over anhydrous sodium sulfate, filtered, and concentrated in vacuo to give a residue. The residue was purified by column chromatography (SiO₂, petroleum ether/ethyl acetate=30/1 to 2/1) to give N-(1-(5-bromothiophen-2-yl)ethyl)-2-methylpropane-2-sulfinamide (12.0 g, 38.7 mmol, 74.5% yield) as a yellow oil. LCMS [M+1]: 309.9.

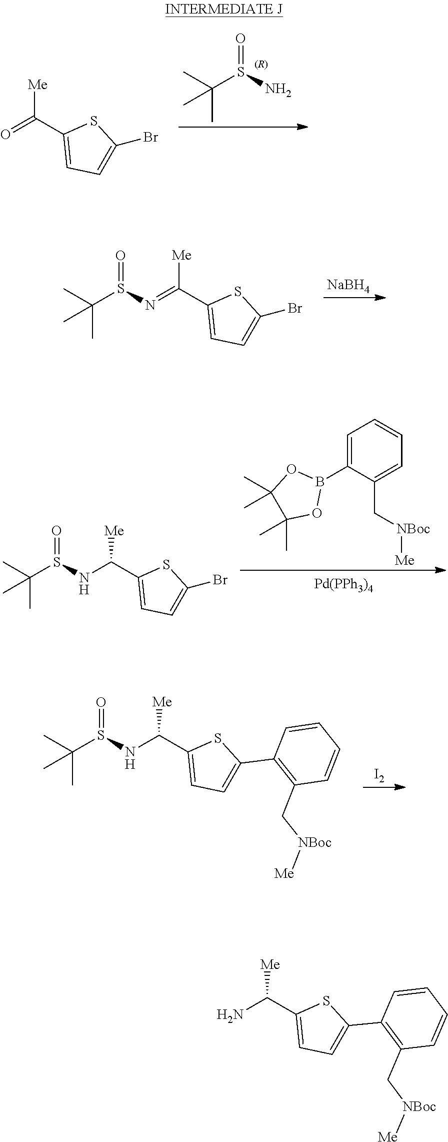

Step A: To a solution of 1-(5-bromothiophen-2-yl)ethan-1-one (10.0 g, 48.8 mmol, 1.00 eq.) and (R)-2-methylpropane-2-sulfinamide (7.68 g, 63.4 mmol, 1.30 eq.) in THF (120 mL) was added titanium (IV) ethoxide (22.3 g, 97.5 mmol, 20.2 mL, 2.00 eq.), the reaction mixture was stirred at 70° C. for 12 hours under a nitrogen atmosphere. The reaction mixture was cooled to 25° C., diluted with water (200 mL) and ethyl acetate (100 mL) to give a suspension, the suspension was filtered and the filtrate was extracted with ethyl acetate (100 mL×3). The combined organic layers were dried over sodium sulfate, filtered, and concentrated under reduced pressure to give (R, E)-N-(1-(5-bromothiophen-2-yl)ethylidene)-2-methylpropane-2-sulfinamide (13.0 g, crude) as a brown oil. LCMS [M+1]: 308.2.

¹H NMR (400 MHz, CDCl₃) δ=7.23 (d, J=4.0 Hz, 1H), 7.04 (d, J=4.0 Hz, 1H), 2.67 (s, 3H), 1.28 (s, 9H).

Step B: To a solution of (R, E)-N-(1-(5-bromothiophen-2-yl)ethylidene)-2-methylpropane-2-sulfinamide (13.0 g, 42.2 mmol, 1.00 eq.) in THF (150 mL) was added sodium borohydride (4.79 g, 127 mmol, 3.00 eq.) at 0° C. The reaction mixture was stirred at 20° C. for 2 hours under a nitrogen atmosphere. Saturate sodium bicarbonate aqueous solution (20.0 mL) was added to the mixture dropwise and diluted with water (200 mL), the resulting aqueous solution was extracted with ethyl acetate (100 mL×3), the combined organic layers were dried over sodium sulfate, filtered, and concentrated under vacuum to give a residue. The residue was purified by column chromatography (SiO₂, petroleum ether/ethyl acetate=30/1 to 2/1) to give (R)—N—((R)-1-(5-bromothiophen-2-yl)ethyl)-2-methylpropane-2-sulfinamide (6.00 g, 17.4 mmol, 41.3% yield, 90.0% purity) as a brown solid. LCMS [M+1]: 309.9.

¹H NMR (400 MHz, CDCl₃) δ=6.90 (d, J=3.6 Hz, 1H), 6.80 (d, J=3.6 Hz, 1H), 4.84-4.66 (m, 1H), 3.50 (d, J=2.8 Hz, 1H), 1.57 (d, J=6.4 Hz, 3H), 1.23 (s, 9H).

Step C: To a solution of (R)—N—((R)-1-(5-bromothiophen-2-yl)ethyl)-2-methylpropane-2-sulfinamide (2.00 g, 6.45 mmol, 1.00 eq.) and tert-butyl methyl(2-(4,4,5,5-tetramethyl-1,3,2-dioxaborolan-2-yl)benzyl)carbamate (2.69 g, 7.74 mmol, 1.20 eq.) in dioxane (20.0 mL) and water (2.00 mL) was added cesium carbonate (6.30 g, 19.3 mmol, 3.00 eq.) and Pd(PPh₃)₄ (745 mg, 645 μmol, 0.10 eq.) under a nitrogen atmosphere. The reaction mixture was stirred at 110° C. for 2 hours under a nitrogen atmosphere. The reaction mixture was then cooled to 25° C., diluted with water (100 mL), and extracted with ethyl acetate (50.0 mL×3). The combined organic layers were dried over sodium sulfate, filtered, and concentrated under reduced pressure to give a residue. The residue was purified by column chromatography (SiO₂, petroleum ether/ethyl acetate=20/1 to 1/1) to give tert-butyl(2-(5-((R)-1-(((R)-tert-butylsulfinyl)amino)ethyl)thiophen-2-yl)benzyl)(methyl)carbamate (2.60 g, 5.19 mmol, 80.6% yield, 90.0% purity) as a yellow oil. LCMS [M+1]: 451.4.

¹H NMR (400 MHZ, CDCl₃) δ=7.40-7.32 (m, 2H), 7.31-7.27 (m, 1H), 7.26-7.22 (m, 1H), 7.01 (s, 1H), 6.83 (s, 1H), 4.95-4.79 (m, 1H), 4.67-4.44 (m, 2H), 3.56 (d, J=3.2 Hz, 1H), 2.93-2.56 (m, 3H), 1.64 (d, J=6.4 Hz, 3H), 1.56-1.36 (m, 9H), 1.26 (s, 9H).

Step D: To a solution of tert-butyl(2-(5-((R)-1-(((R)-tert-butylsulfinyl)amino)ethyl)thiophen-2-yl)benzyl)(methyl)carbamate (2.60 g, 5.77 mmol, 1.00 eq.) in THF (20.0 mL) and water (4.00 mL) was added iodine (439 mg, 1.73 mmol, 349 μL, 0.30 eq.), the reaction mixture was stirred at 50° C. for 2 hours. The reaction mixture was cooled to 25° C., diluted with saturate sodium bicarbonate (50.0 mL) and extracted with ethyl acetate (20.0 mL×3). The combined organic layers were dried over sodium sulfate, filtered, and concentrated under reduced pressure to give a residue. The residue was purified by column chromatography (SiO₂, petroleum ether/ethyl acetate=10/1 to 0/1) to give (R)-tert-butyl (R)-(2-(5-(1-aminoethyl)thiophen-2-yl)benzyl)(methyl)carbamate (1.50 g, 3.68 mmol, 63.8% yield, 85.0% purity) as a yellow oil. LCMS [2M+1]: 693.3.

¹H NMR (400 MHZ, CDCl₃) δ=7.39-7.31 (m, 2H), 7.30-7.20 (m, 2H), 7.01 (d, J=2.8 Hz, 1H), 6.81 (d, J=3.2 Hz, 1H), 4.61-4.48 (m, 3H), 4.04 (s, 2H), 2.73 (s, 3H), 1.64 (d, J=6.4 Hz, 3H), 1.57-1.33 (m, 9H).

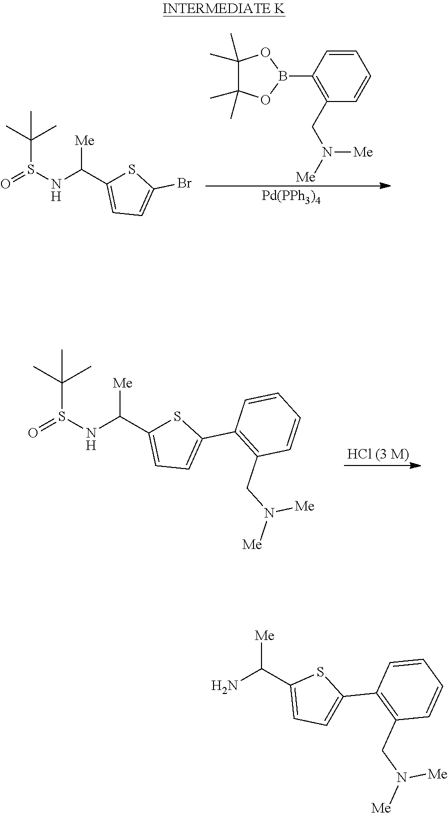 [SMILES: CC(NS(=O)C(C)(C)C)c1ccc(Br)s1 ; CN(C)Cc1ccccc1B1OC(C)(C)C(C)(C)O1 ; CC(NS(=O)C(C)(C)C)c1ccc(-c2ccccc2CN(C)C)s1 ; "HCl (3 M)" ; CC(N)c1ccc(-c2ccccc2CN(C)C)s1 ; "Pd(PPh3)4"]

Step A: To a solution of N-(1-(5-bromothiophen-2-yl)ethyl)-2-methylpropane-2-sulfinamide (0.50 g, 1.61 mmol, 1.00 eq.) and N, N-dimethyl-1-(2-(4,4,5,5-tetramethyl-1,3,2-dioxaborolan-2-yl)phenyl) methanamine (505 mg, 1.93 mmol, 1.20 eq.) in dioxane (5.00 mL) and water (0.50 mL) was added cesium carbonate (1.58 g, 4.83 mmol, 3.00 eq.) and Pd(PPh₃)₄ (186 mg, 161 μmol, 0.10 eq.), then degassed and purged with nitrogen 3 times. The reaction mixture was stirred at 110° C. for 2 hours under a nitrogen atmosphere. Upon completion, the reaction mixture was cooled to 25° C., diluted with water (50.0 mL) and extracted with ethyl acetate (20.0 mL×3). The combined organic layers were dried over anhydrous sodium sulfate, filtered, and concentrated under reduced pressure to give a residue. The residue was purified by column chromatography (SiO₂, petroleum ether/ethyl acetate=20/1 to 0/1) to give N-(1-(5-(2-((dimethylamino)methyl)phenyl)thiophen-2-yl)ethyl)-2-methylpropane-2-sulfinamide (450 mg, 1.15 mmol, 71.3% yield, 93.0% purity) as a brown oil. LCMS [M+1]: 365.2.

Step B: To a solution of N-(1-(5-(2-((dimethylamino)methyl)phenyl)thiophen-2-yl)ethyl)-2-methylpropane-2-sulfinamide (410 mg, 1.12 mmol, 1.00 eq.) in THF (4.00 mL) was added hydrochloric acid (3.00 M, 375 μL, 1.00 eq.), the reaction mixture was stirred at 20° C. for 2 hours. Upon completion, the reaction mixture was diluted with saturated sodium bicarbonate (50.0 mL) and extracted with ethyl acetate (20.0 mL×3). The combined organic layers were dried over anhydrous sodium sulfate, filtered, and concentrated under reduced pressure to give a residue. The residue was purified by column chromatography (SiO₂, petroleum ether/ethyl acetate=10/1 to dichloromethane/methanol=10/1) to give 1-(5-(2-((dimethylamino)methyl)phenyl)thiophen-2-yl)ethanamine (200 mg, 691 μmol, 61.5% yield, 90.0% purity) as a yellow oil.

¹H NMR (400 MHZ, DMSO-d₆) δ=7.48-7.42 (m, 1H), 7.41-7.36 (m, 1H), 7.34-7.28 (m, 2H), 7.13 (d, J=3.6 Hz, 1H), 6.96-6.92 (m, 1H), 4.29-4.21 (m, 1H), 3.39 (s, 2H), 2.14 (s, 6H), 1.38 (d, J=6.4 Hz, 3H).

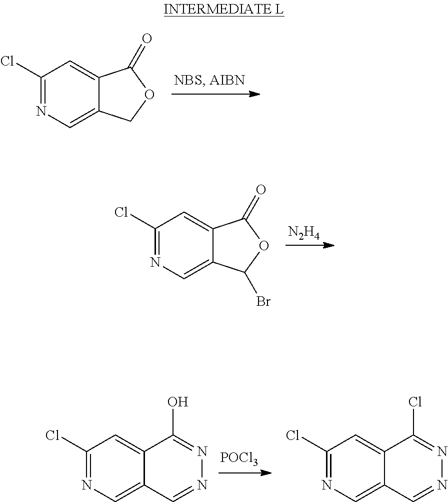

Step A: To a solution of 6-chlorofuro[3,4-c]pyridin-1(3H)-one (1.50 g, 8.85 mmol, 1.00 eq.) in carbon tetrachloride (10.0 mL) was added AIBN (145 mg, 884 μmol, 0.10 eq.) and NBS (1.42 g, 7.96 mmol, 0.9 eq.). The reaction mixture was stirred at 80° C. for 12 hours. The reaction was filtered and the filtrate was concentrated under vacuum to give a residue. The residue was purified by column chromatography (SiO₂, petroleum ether/ethyl acetate=50/1 to 10/1) to give 3-bromo-6-chlorofuro[3,4-c]pyridin-1(3H)-one (1.20 g, 4.83 mmol, 54.6% yield) as yellow oil. LCMS [M+3]: 249.8.

¹H NMR (400 MHZ, CDCl₃) δ=8.84-8.80 (m, 1H), 7.84 (s, 1H), 7.47 (s, 1H).

Step B: To a solution of 3-bromo-6-chlorofuro[3,4-c]pyridin-1(3H)-one (1.20 g, 4.83 mmol, 1.00 eq.) in ethanol (20.0 mL) was added hydrazine hydrate (370 mg, 7.24 mmol, 359 μL, 1.50 eq.) at 0° C. The reaction mixture was stirred at 80° C. for 30 minutes. The reaction was cooled to 25° C., poured into ice water (1.00 mL) to give a suspension. The suspension was filtered, and the filter cake was collected and dried under vacuum to give 7-chloropyrido[3,4-d]pyridazin-1-ol (800 mg, 4.41 mmol, 91.2% yield) as a yellow solid. LCMS [M+1]⁺: 182.0.

¹H NMR (400 MHZ, DMSO-d₆) δ=13.08 (br s, 1H), 9.20 (s, 1H), 8.53 (s, 1H), 8.10 (s, 1H).

Step C: To a solution of 7-chloropyrido[3,4-d]pyridazin-1-ol (78.0 mg, 430 μmol, 1.00 eq.) in acetonitrile (2.00 mL) was added phosphorus (V) oxychloride (231 mg, 1.50 mmol, 139 μL, 3.50 eq.) at 25° C. The reaction mixture was stirred at 80° C. for 2 hours. The reaction was cooled at 25° C., poured into saturated sodium bicarbonate aqueous solution (2.00 mL) and stirred for 5 minutes at 0° C. The aqueous phase was extracted with ethyl acetate (3.00 mL×3). The combined organic phases were washed with brine (2.00 mL×3), dried over anhydrous sodium sulfate, filtered, and concentrated in vacuo to give 1,7-dichloropyrido[3,4-d]pyridazine (65.0 mg, crude) as a red solid. LCMS [M+1]: 199.8.

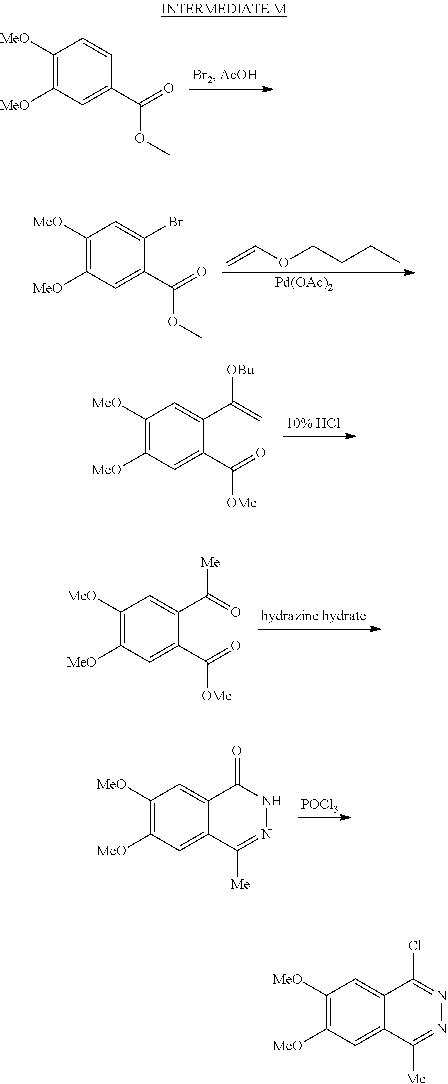

Step A: To a mixture of methyl 3,4-dimethoxybenzoate (10.0 g, 51.0 mmol, 1.00 eq.) in acetic acid (50.0 mL) was added bromine (8.96 g, 56.1 mmol, 2.89 mL, 1.10 eq.) in acetic acid (50.0 mL) at 0° C. over 1.5 hours. The mixture was then slowly brought to room temperature and stirred for 45 minutes. Upon completion, the reaction was quenched by pouring into water (700 mL) and stirred for 30 minutes, then stirring was stopped and the mixture was filtered after 1 hr of sitting. The collected solid was washed with water (100 mL) and washed with sodium sulfite aqueous solution (100 mL). The solid was partially dried, dissolved in hot methanol (300 mL), and the resultant solution was cooled. The cool methanolic solution was treated with water (200 mL) to give a suspension, the suspension was filtered, the filter cake was collected and dried in vacuo to give methyl 2-bromo-4,5-dimethoxybenzoate (9.00 g, 32.7 mmol, 64.2% yield) as a white powder. LCMS [M+1]: 275.3.

¹H NMR (400 MHZ, DMSO-d₆) δ=7.36 (s, 1H), 7.24 (s, 1H), 3.84 (s, 3H), 3.82 (s, 3H), 3.79 (s, 3H).

Step B: A mixture of methyl 2-bromo-4,5-dimethoxy-benzoate (6.00 g, 21.8 mmol, 1.00 eq.), 1-(vinyloxy) butane (10.9 g, 109 mmol, 14.0 mL, 5.00 eq.), Pd(OAc)2 (490 mg, 2.18 mmol, 0.10 eq.), triphenylphosphine (1.14 g, 4.36 mmol, 0.20 eq.) and triethylamine (2.65 g, 26.2 mmol, 3.64 mL, 1.20 eq.) in acetonitrile (60.0 mL) was degassed and purged with nitrogen 3 times, and then the reaction mixture was stirred at 100° C. for 16 hours under a nitrogen atmosphere. The mixture was then cooled to 25° C., filtered, and the filtrate concentrated under reduced pressure to give methyl methyl 2-(1-butoxyvinyl)-4,5-dimethoxybenzoate (6.00 g, crude) was obtained as a yellow oil which was used in the next step directly.

Step C: A mixture of methyl 2-(1-butoxyvinyl)-4,5-dimethoxybenzoate (6.00 g, 20.4 mmol, 1.00 eq.) in hydrochloric acid (10% in water, 61.2 g, 168 mmol, 60.0 mL, 8.23 eq.) and THF (60.0 mL) was stirred at 20° C. for 1 hour. The reaction mixture was diluted with water (100 mL) and extracted with ethyl acetate (50.0 mL×3). The combined organic layers were brought to pH=7 with a saturated sodium bicarbonate aqueous solution, then the organic layers were washed with brine (100 mL), dried over anhydrous sodium sulfate, filtered, and concentrated under reduced pressure to give a residue. The residue was triturated with petroleum ether/ethyl acetate=5/1 (50.0 mL) at 20° C. for 20 minutes to give a suspension, the suspension was filtered, the filter cake was collected and dried in vacuo to give methyl 2-acetyl-4,5-dimethoxybenzoate (3.00 g, 12.6 mmol, 61.8% yield) as a white solid.

¹H NMR (400 MHZ, DMSO-d6) δ=7.26 (s, 1H), 7.17 (s, 1H), 3.86 (s, 3H), 3.84 (s, 3H), 3.77 (s, 3H), 2.46 (s, 3H).

Step D: To a solution of methyl 2-acetyl-4,5-dimethoxybenzoate (3.00 g, 12.6 mmol, 1.00 eq.) in ethanol (30.0 mL) was added hydrazine hydrate (2.22 g, 37.8 mmol, 2.16 mL, 3.00 eq.) at room temperature, and then the reaction mixture was stirred at 95° C. for 30 minutes. The reaction mixture was diluted with water (100 mL) and extracted with ethyl acetate several times. The combined organic layers were washed with brine (100 mL), dried over anhydrous sodium sulfate, filtered, and concentrated under reduced pressure to give a residue. The residue was triturated with ethyl acetate (50.0 mL) at 20° C. for 20 minutes to give a suspension, the suspension was filtered, the filter cake was collected and dried in vacuo to give 6,7-dimethoxy-4-methylphthalazin-1(2H)-one (2.00 g, 9.08 mmol, 72.1% yield) as a off-white solid. LCMS [M+1]: 221.4.

¹H NMR (400 MHZ, DMSO-d6) δ=12.25 (s, 1H), 7.58 (s, 1H), 7.21 (s, 1H), 3.96 (s, 3H), 3.92 (s, 3H), 2.48 (s, 3H).

Step E: A mixture of 6,7-dimethoxy-4-methylphthalazin-1(2H)-one (1.30 g, 5.90 mmol, 1.00 eq.) in phosphorus (V) oxychloride (13.0 mL) was stirred at 120° C. for 12 hours. The reaction mixture was concentrated under reduced pressure to give 1-chloro-6,7-dimethoxy-4-methylphthalazine (1.20 g, crude) as a yellow solid. LCMS [M+1]: 239.0.

¹H NMR (400 MHZ, DMSO-d6) δ=7.80 (s, 1H), 7.64 (s, 1H), 4.13 (s, 3H), 4.12 (s, 3H), 3.08 (s, 3H).

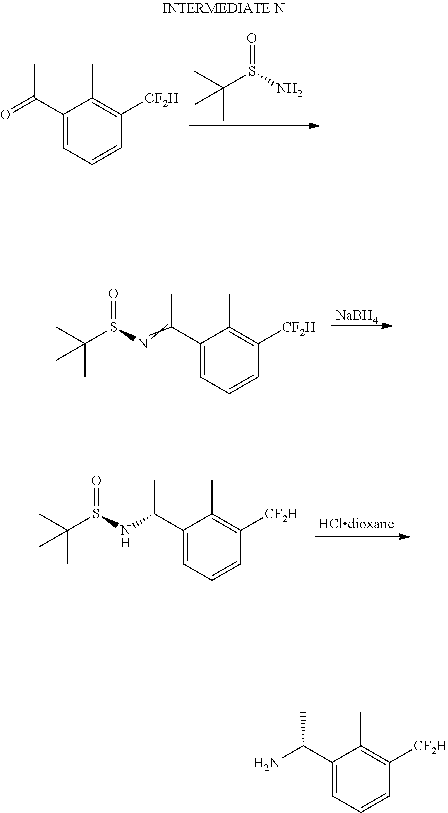

Step A: To a solution of 1-(3-(difluoromethyl)-2-methylphenyl)ethan-1-one (0.37 g, 1.99 mmol, 1.00 eq.) in tetrahydrofuran (10.0 mL) was added titanium (IV) ethoxide (2.27 g, 9.95 mmol, 2.06 mL, 5.00 eq.) and (R)-2-methylpropane-2-sulfinamide (724 mg, 5.97 mmol, 3.00 eq.). The mixture was stirred at 75° C. for 16 hours. The reaction mixture was quenched by addition saturated aqueous sodium bicarbonate 20.0 mL at 25° C. The mixture was filtered, and filtrate was extracted with ethyl acetate 45.0 mL (15.0 mL×3). The combined organic layers were washed with brine 20.0 mL (20.0 mL×1), dried over anhydrous sodium sulfate, filtered, and concentrated under reduced pressure to give a residue. The residue was purified by flash silica gel chromatography (0˜12% Ethyl acetate/Petroleum ether) to give (R,E)-N-(1-(3-(difluoromethyl)-2-methylphenyl)ethylidene)-2-methylpropane-2-sulfinamide (0.36 g, 1.19 mmol, 59.8% yield, 95.0% purity) as a colorless oil.

¹H NMR (400 MHZ, CD₃OD) δ=7.55-7.62 (m, 1H), 7.16-7.51 (m, 2H), 6.79-7.13 (m, 1H), 2.48-2.73 (m, 3H), 2.27-2.47 (m, 3H), 1.19-1.30 (m, 9H).

Step B: To a solution of (R,E)-N-(1-(3-(difluoromethyl)-2-methylphenyl)ethylidene)-2-methylpropane-2-sulfinamide (340 mg, 1.18 mmol, 1.00 eq.) in tetrahydrofuran (5.00 mL) was added sodium borohydride (89.5 mg, 2.37 mmol, 2.00 eq.). The mixture was stirred at 0° C. for 1 hour. The reaction mixture was quenched by addition water 10.0 mL at 25° C., and then extracted with ethyl acetate 30.0 mL (10.0 mL×3). The combined organic layers were washed with brine (10.0 mL×1) dried over anhydrous sodium sulfate, filtered, and concentrated under reduced pressure to give a residue. The residue was purified by flash silica gel chromatography (0-13% Ethyl acetate/Petroleum ether) to give (R)—N—((R)-1-(3-(difluoromethyl)-2-methylphenyl)ethyl)-2-methylpropane-2-sulfinamide (190 mg, 643 μmol, 54.4% yield, 98.0% purity) as a yellow oil. LCMS [M+1]⁺=290.1.

Step C: A mixture of (R)—N—((R)-1-(3-(difluoromethyl)-2-methylphenyl)ethyl)-2-methylpropane-2-sulfinamide (140 mg, 484 μmol, 1.00 eq.) in dioxane hydrochloride (4.00 M, 7.00 mL, 57.9 eq) was stirred at 25° C. for 1 h. The reaction mixture was concentrated under reduced pressure to give crude product (R)-1-(3-(difluoromethyl)-2-methylphenyl)ethan-1-amine (110 mg, 475 μmol, 98.2% yield, 80.0% purity) as a white solid, which was used without further purification. LCMS [M+1]⁺=186.0.

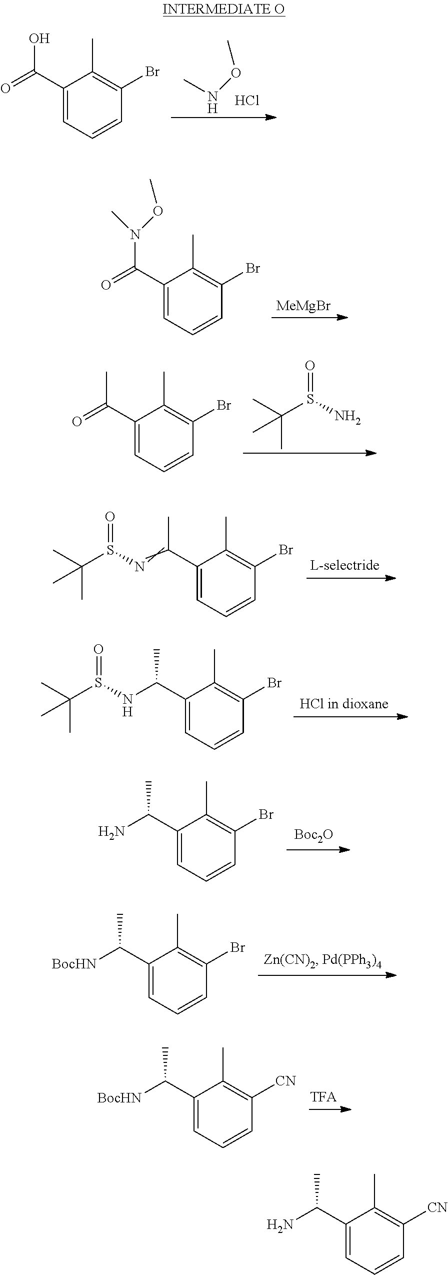

Step A: To a solution of 3-bromo-2-methylbenzoic acid (100 g, 465 mmol, 1.00 eq.) and N,O-dimethylhydroxylamine hydrochloride (68.6 g, 512 mmol, 1.10 eq., HCl) in DMF (1000 mL) was added 1-[Bis(dimethylamino)methylene]-1H-1,2,3-triazolo[4,5-b]pyridinium 3-oxid hexafluorophosphate (195 g, 512 mmol, 1.10 eq.) and N,N-diisopropylethylamine (180 g, 1.40 mol, 243 mL, 3.00 eq.). The mixture was stirred at 25° C. for 2 hours, then poured into water (1000 mL) and stirred for 15 minutes. The aqueous phase was extracted with ethyl acetate (1000 mL×3). The combined organic phases were washed with brine (1000 mL×5), dried over anhydrous sodium sulfate, filtered and concentrated in vacuo to give 3 3-bromo-N-methoxy-N,2-dimethylbenzamide (120 g, crude) as yellow oil. LCMS [M+1]⁺: 258.0.

Step B: To a solution of 33-bromo-N-methoxy-N,2-dimethylbenzamide (120 g, 465 mmol, 1.00 eq.) in THF (100 mL) was added methyl magnesium bromide (3.0 M, 180 mL, 1.16 eq.) at 0° C. The mixture was stirred between 0-40° C. for 3 hours, then the mixture was cooled to 0° C. and hydrochloric acid (6.0 N) (450 mL) was added dropwise, and stirred for 2 hours between 40-45° C. Then the mixture was cooled to 25° C. and poured into a saturated ammonium chloride solution (9000 mL). The aqueous phase was extracted with ethyl acetate (1500 mL×3). The combined organic phase was washed with brine (1000 mL×3), dried over anhydrous sodium sulfate, filtered, and concentrated under vacuum to give 1-(3-bromo-2-methylphenyl)ethan-1-one (90.0 g, 422 mmol, 90.9% yield) as yellow oil.

¹H NMR (400 MHZ, CD₃OD) δ=7.70 (dd, J=1.2, 8.0 Hz, 1H), 7.62 (dd, J=0.8, 7.6 Hz, 1H), 7.19 (t, J=8.0 Hz, 1H), 2.56 (s, 3H), 2.46 (s, 3H).

Step C: To a solution of 1-(3-bromo-2-methylphenyl)ethan-1-one (88.0 g, 413 mmol, 1.00 eq.) and (S)-2-methylpropane-2-sulfinamide (60.1 g, 496 mmol, 1.20 eq.) in THF (100 mL) was added titanium (IV) ethoxide (471 g, 2.07 mol, 428 mL, 5.00 eq.) and diglyme (55.4 g, 413 mmol, 59.1 mL, 1.00 eq.). The mixture was stirred at 80° C. for 2 hours then poured into water (300 mL) and stirred for 15 minutes. The mixture was then filtered and concentrated in vacuo to give a residue. The residue was purified by column chromatography (SiO₂, petroleum ether/ethyl acetate=100/1 to 40/1) to give (S)—N-(1-(3-bromo-2-methylphenyl)ethylidene)-2-methylpropane-2-sulfinamide (110 g, 348 mmol, 84.2% yield) as yellow oil.

¹H NMR (400 MHZ, CD₃OD) δ=7.63 (br t, J=6.8 Hz, 2H), 7.28 (br d, J=7.6 Hz, 1H), 7.17 (t, J=8.0 Hz, 2H), 7.14-7.02 (m, 1H), 2.67 (s, 3H), 2.50 (br d, J=4.8 Hz, 3H), 2.42 (s, 3H), 2.31 (br d, J=17.2 Hz, 3H), 1.31-1.26 (m, 9H), 1.24-1.16 (m, 9H)

Step D: To a solution of (S)—N-(1-(3-bromo-2-methylphenyl)ethylidene)-2-methylpropane-2-sulfinamide (109 g, 345 mmol, 1.00 eq.) in THF (1100 mL) was added L-selectride (1.0 M, 689 mL, 2.00 eq.) at −78° C. The mixture was stirred at −78° C. for 2 hours then poured into a saturated aqueous solution of ammonium chloride (1000 mL) and stirred for 60 minutes at 25° C. The aqueous phase was extracted with ethyl acetate (1000 mL×3). The combined organic phase were washed with brine (500 mL×3), dried over anhydrous sodium sulfate, filtered, and concentrated under vacuum to give a residue. The residue was purified by column chromatography (SiO₂, petroleum ether/ethyl acetate=100/1 to 2/1) to give a residue. The residue was further washed with petroleum ether to give (S)—N—((R)-1-(3-bromo-2-methylphenyl)ethyl)-2-methylpropane-2-sulfinamide (70.0 g, 220 mmol, 63.8% yield) as a white solid. LCMS [M+1]⁺: 318.1.

Step E: To a solution of (S)—N—((R)-1-(3-bromo-2-methylphenyl)ethyl)-2-methylpropane-2-sulfinamide (71.0 g, 223 mmol, 1.00 eq.) in an HCl/dioxane solution (300 mL) and MeOH (300 mL) was stirred at 0° C. for 30 minutes. The mixture was concentrated in vacuo to give a (R)-1-(3-bromo-2-methylphenyl)ethan-1-amine (55.0 g, crude, HCl) as a white solid. LCMS [M+1]⁺: 214.1.

Step F: To a solution of (R)-1-(3-bromo-2-methylphenyl)ethan-1-amine (55.0 g, 220 mmol, 1.00 eq., HCl) and Boc₂O (48.4 g, 222 mmol, 50.9 mL, 1.01 eq.) in dichloromethane (500 mL) was added N,N-diisopropylethylamine (56.7 g, 439 mmol, 76.5 mL, 2.00 eq.). The mixture was stirred between 0-25° C. for 30 minutes, then concentrated under vacuum to give a residue. The residue was purified by column chromatography (SiO₂, petroleum ether/ethyl acetate=1/0 to 100/1) to give a residue. The residue was further washed with petroleum ether to give tert-butyl (R)-(1-(3-bromo-2-methylphenyl)ethyl)carbamate (51.0 g, 162 mmol, 73.9% yield) as a white solid. LCMS [M−55]⁻: 258.0.

¹H NMR (400 MHZ, CD₃OD) δ=7.43 (d, J=8.0 Hz, 1H), 7.32 (d, J=8.0 Hz, 1H), 7.10-7.03 (m, 1H), 4.93 (br d, J=6.4 Hz, 2H), 2.45 (s, 3H), 1.41 (br s, 9H), 1.33 (d, J=6.8 Hz, 3H).

Step G: To a solution of tert-butyl (R)-(1-(3-bromo-2-methylphenyl)ethyl)carbamate (51.0 g, 162 mmol, 1.00 eq.) in DMF (540 mL) was added zinc cyanide (22.9 g, 195 mmol, 12.4 mL, 1.20 eq.) and Pd(PPh₃)₄ (18.8 g, 16.2 mmol, 0.10 eq.). The mixture was stirred at 110° C. for 3 hours, then cooled to 25° C. and poured into water (500 mL). The aqueous phase was extracted with ethyl acetate (100 mL×3). The combined organic phases were washed with brine (1000 mL×3), dried over anhydrous sodium sulfate, filtered, and concentrated under vacuum to give a residue. The residue was purified by column chromatography (SiO₂, petroleum ether/ethyl acetate=100/1 to 5/1) to give tert-butyl (R)-(1-(3-cyano-2-methylphenyl)ethyl)carbamate (37.0 g, 142.1 mmol, 87.6% yield) as a white solid. LCMS [M−55]⁺: 205.0.

¹H NMR (400 MHZ, CD₃OD) δ=7.63 (d, J=7.6 Hz, 1H), 7.54 (d, J=7.2 Hz, 1H), 7.39-7.30 (m, 1H), 4.93 (br d, J=6.8 Hz, 1H), 2.58 (s, 3H), 1.40 (br s, 9H), 1.34 (d, J=7.2 Hz, 3H).

Step H: To a solution of tert-butyl (R)-(1-(3-cyano-2-methylphenyl)ethyl)carbamate (49.0 g, 188 mmol, 1.00 eq.) in dichloromethane (400 mL) was added TFA (133 mL). The mixture was stirred at 0° C. for 30 minutes then poured into saturated sodium bicarbonate solution (200 mL) and stirred for and additional 30 minutes. The aqueous phase was extracted with ethyl acetate (1000 mL×3). The combined organic phases were washed with brine (200 mL×3), dried over anhydrous sodium sulfate, filtered, and concentrated under vacuum to give (R)-3-(1-aminoethyl)-2-methylbenzonitrile (26.0 g, 162 mmol, 86.2% yield) as yellow oil. LCMS [M−16]⁺: 144.1.

¹H NMR (400 MHZ, DMSO-d₆) δ=8.36 (br s, 2H), 7.86 (d, J=8.0 Hz, 1H), 7.80 (dd, J=0.8, 7.6 Hz, 1H), 7.51 (t, J=8.0 Hz, 1H), 4.68 (q, J=6.8 Hz, 1H), 2.55 (s, 3H), 1.48 (d, J=6.8 Hz, 3H).

SFC conditions: Column: Chiralpak IC-3 50×4.6 mm I.D., 3 μm Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35° C.; Back Pressure: 100 Bar.

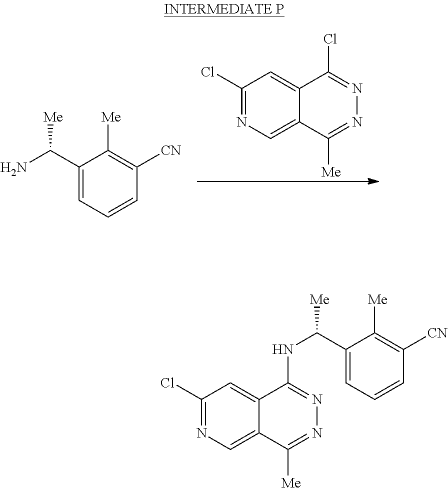 [SMILES: Cc1c(C#N)cccc1[C@@H](C)N ; Cc1nnc(Cl)c2cc(Cl)ncc12 ; Cc1c(C#N)cccc1[C@@H](C)Nc1nnc(C)c2cnc(Cl)cc12]

To a solution of (R)-3-(1-aminoethyl)-2-methylbenzonitrile (16.0 g, 99.9 mmol, 1.00 eq.) and 1,7-dichloro-4-methylpyrido[3,4-d]pyridazine (21.4 g, 99.9 mmol, 1.00 eq.) in DMSO (130 mL) was added cesium fluoride (22.8 g, 150 mmol, 5.52 mL, 1.50 eq.), and the mixture was stirred at 130° C. for 2 hours. The mixture was then cooled to 25° C., diluted with water (200 mL), and extracted with ethyl acetate (200 mL×3). The combined organic phases were washed with brine (100 mL×3), dried over anhydrous sodium sulfate, filtered, and concentrated under reduced pressure. The residue was purified by prep-HPLC [column: Kromasil Eternity XT 250×80 mm×10 μm; mobile phase: phase A: water (0.1% TFA), phase B: acetonitrile; B %: 25%-55%]. To the combined fractions were combined and the pH was adjusted to pH=8 using aqueous sodium bicarbonate. The suspension was extracted with ethyl acetate (1000 mL×3), and the combined organic phases were washed with brine (100 mL×3), dried over anhydrous sodium sulfate, filtered, and concentrated under reduced pressure to give (R)-3-(1-((7-chloro-4-methylpyrido[3,4-d]pyridazin-1-yl)amino)ethyl)-2-methylbenzonitrile (14.5 g, 42.9 mmol, 43.0% yield) as a yellow solid.

¹H NMR (400 MHZ, CDCl₃) δ=9.19 (d, J=0.4 Hz, 1H), 7.74 (s, 1H), 7.63 (d, J=8.0 Hz, 1H), 7.50 (dd, J=1.2, 7.6 Hz, 1H), 7.23 (t, J=7.6 Hz, 1H), 5.72 (quin, J=6.8 Hz, 1H), 5.40 (br d, J=6.0 Hz, 1H), 2.86 (s, 3H), 2.69 (s, 3H), 1.63 (s, 3H).

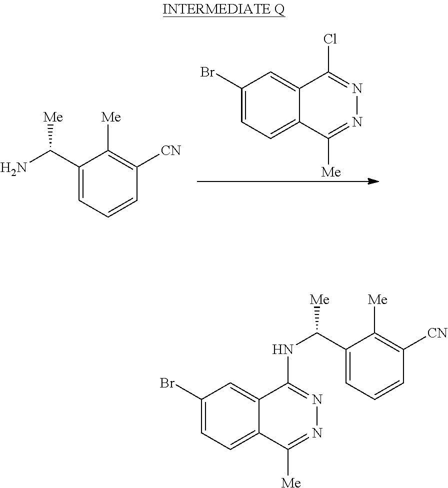 [SMILES: Cc1c(C#N)cccc1[C@@H](C)N ; Cc1nnc(Cl)c2cc(Br)ccc12 ; Cc1c(C#N)cccc1[C@@H](C)Nc1nnc(C)c2ccc(Br)cc12]

To a solution of (R)-3-(1-aminoethyl)-2-methylbenzonitrile (5.32 g, 19.4 mmol, 1.00 eq., TFA) and 6-bromo-4-chloro-1-methylphthalazine (5.00 g, 19.4 mmol, 1.00 eq.) in DMSO (30.0 mL) was added cesium fluoride (5.90 g, 38.8 mmol, 1.43 mL, 2.00 eq.) and N,N-diisopropylethylamine (5.02 g, 38.8 mmol, 6.76 mL, 2.00 eq.), and the mixture was stirred at 130° C. for 2 hours. The mixture was then cooled to 25° C., diluted with water (10.0 mL), and the aqueous phase was extracted with ethyl acetate (100 mL×3). The combined organic phases were washed with brine (100 mL×3), dried over anhydrous sodium sulfate, filtered, and concentrated under reduced pressure. The residue was purified by column chromatography (SiO₂, petroleum ether/ethyl acetate=20/1 to 2/1) to give (R)-3-(1-((7-bromo-4-methylphthalazin-1-yl)amino)ethyl)-2-methylbenzonitrile (5.20 g, 13.6 mmol, 70.2% yield) as a yellow solid. LCMS [M+1]⁺: 381.1.

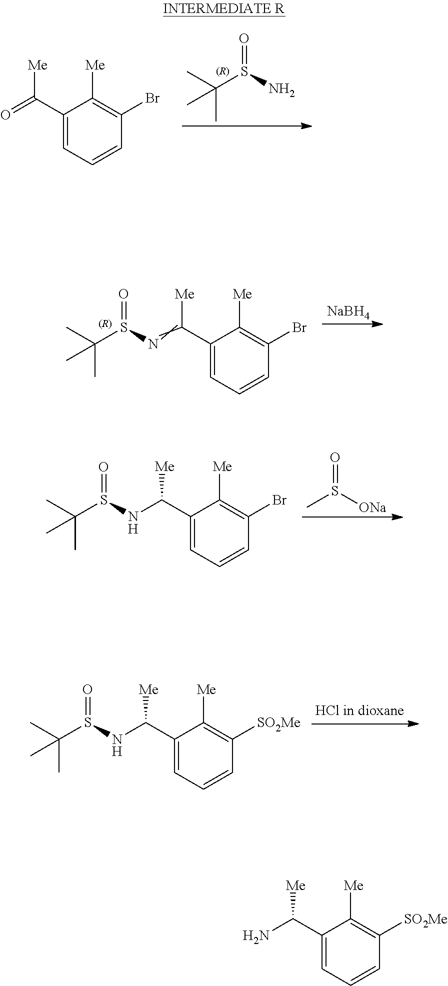

Step A: A mixture of (R)-2-methylpropane-2-sulfinamide (5.12 g, 42.2 mmol, 1.00 eq.), 1-(3-bromo-2-methylphenyl)ethan-1-one (9.00 g, 42.2 mmol, 1.00 eq.), titanium (IV) isopropoxide (60.0 g, 211 mmol, 62.3 mL, 5.00 eq.) in THF (90.0 mL) was degassed and purged with nitrogen 3 times, and stirred at 80° C. for 12 hours. The mixture was cooled to 25° C., quenched by addition of water (100 mL), filtered, and the filtrate was partitioned between ethyl acetate (300 mL) and water (300 mL). The organic phase was dried over sodium sulfate, filtered, and concentrated under reduced pressure to give a residue. The residue was purified by column chromatography (SiO₂, petroleum ether/ethyl acetate=1/0 to 5/1) (R)—N-(1-(3-bromo-2-methylphenyl)ethylidene)-2-methylpropane-2-sulfinamide (7.23 g, 22.8 mmol, 54.1% yield) as a yellow solid. LCMS [M+3]⁺: 318.0.

¹H NMR (400 MHZ, CD₃OD) δ=7.67-7.58 (m, 2H), 7.28 (br d, J=7.6 Hz, 1H), 7.17 (t, J=8.0 Hz, 2H), 7.14-7.01 (m, 1H), 2.67 (s, 3H), 2.50 (br d, J=4.0 Hz, 3H), 2.42 (s, 3H), 2.31 (br d, J=17.2 Hz, 3H), 1.28 (s, 9H), 1.21 (br d, J=11.2 Hz, 9H). (the ratio of E/Z isomers was ˜1/1).

Step B: To a solution of (R)—N-(1-(3-bromo-2-methylphenyl)ethylidene)-2-methylpropane-2-sulfinamide (400 mg, 1.26 mmol, 1.00 eq.) in THF (5.00 mL) was added sodium borohydride (239 mg, 6.32 mmol, 5.00 eq.) at 0° C. portionwise, then the reaction was stirred at 25° C. for 1 hour. The reaction mixture was poured into water (30.0 mL) and stirred for 5 minutes. The resulting aqueous phase was extracted with ethyl acetate (150 mL×3), and the combined organic phases were washed with brine (150 mL×3), dried over anhydrous sodium sulfate, filtered, and concentrated under reduced pressure. The residue was purified by column chromatography (SiO₂, petroleum ether/ethyl acetate=10/1 to 1/1) to give (R)—N—((R)-1-(3-bromo-2-methylphenyl)ethyl)-2-methylpropane-2-sulfinamide (200 mg, 628 μmol, 49.7% yield) as a brown oil.

Step C: To a mixture of (R)—N—((R)-1-(3-bromo-2-methylphenyl)ethyl)-2-methylpropane-2-sulfinamide (250 mg, 786 μmol, 1.00 eq.), sodium methanesulfinate (176 mg, 1.73 mmol, 2.20 eq.), potassium carbonate (326 mg, 2.36 mmol, 3.00 eq.) and L-proline (18.1 mg, 157 μmol, 0.20 eq.) in dimethyl sulfoxide (3.00 mL) was added copper (I) iodide (15.0 mg, 78.6 μmol, 0.10 eq.) at 20° C., the mixture was stirred at 130° C. for 3 hours under a nitrogen atmosphere. To the mixture was added water (15.0 mL), and the mixture was extracted with ethyl acetate (20.0 mL×3). The combined organic phases were washed with brine (30.0 mL×3), dried over anhydrous sodium sulfate, filtered, and concentrated under reduced pressure to give a residue. The residue was purified by prep-TLC (silica gel plate, petroleum ether/ethyl acetate=1/1) to give (R)-2-methyl-N—((R)-1-(2-methyl-3-(methylsulfonyl)phenyl)ethyl)propane-2-sulfinamide (120 mg, 378 μmol, 48.1% yield) as a yellow oil. LCMS [M+1]⁺: 318.1.

¹H NMR (400 MHZ, DMSO-d₆)>=7.85 (dd, J=8.0, 1.2 Hz, 1H), 7.78 (d, J=7.6 Hz, 1H), 7.46 (t, J=8.0 Hz, 1H), 5.42-5.50 (m, 1H), 4.71-4.80 (m, 1H), 3.22 (s, 3H), 2.65 (s, 3H), 1.46 (d, J=6.8 Hz, 3H), 1.09 (s, 9H).

Step D: A mixture of (R)-2-methyl-N—((R)-1-(2-methyl-3-(methylsulfonyl)phenyl)ethyl)propane-2-sulfinamide (120 mg, 378 μmol, 1.00 eq.) in hydrochloric acid (4.0 M in dioxane, 2.00 mL, 21.2 eq.) was stirred at 20° C. for 1 hour. The mixture was concentrated under reduced pressure to give (R)-1-(2-methyl-3-(methylsulfonyl)phenyl)ethan-1-amine (91.0 mg, crude, HCl) as a white solid.

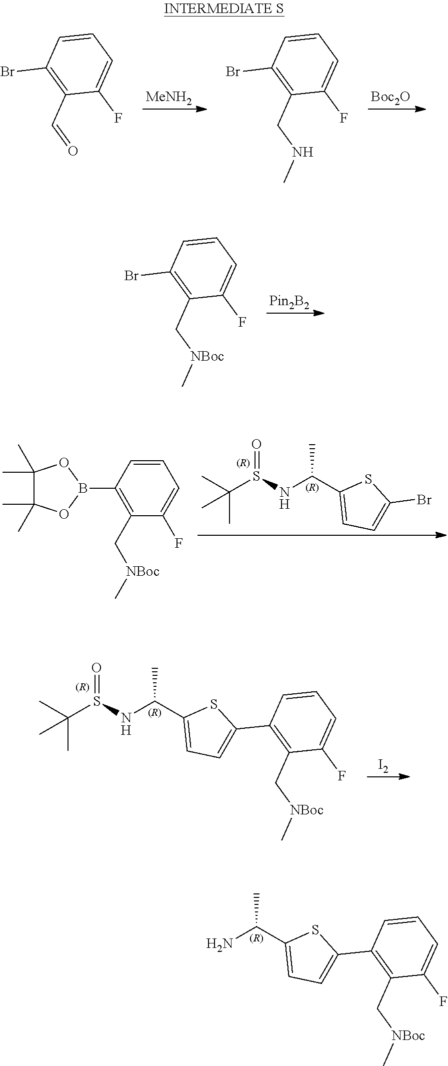

Step A: To a solution of methyl amine (100 g, 1.48 mol, 3.01 eq. HCl salt) in THF (1.00 L) was added N, N-diisopropylethylamine (237 g, 1.84 mol, 3.73 eq.), 2-bromo-6-fluorobenzaldehyde (100 g, 493 mmol, 1.00 eq.), acetic acid (9.00 g, 150 mmol, 0.30 eq.) and sodium cyanoborohydride (62.0 g, 987 mmol, 2.00 eq.). The reaction mixture was stirred at 25° C. for 3 hours, then diluted with water (500 mL) and extracted with ethyl acetate (1.00 L×2). The combined organic phases were washed with brine (500 mL), dried over sodium sulfate, filtered, and concentrated under vacuum to give 1-(2-bromo-6-fluorophenyl)-N-methylmethanamine (120 g, 484 mmol, 88% purity) as off white solid which was used in the next step directly. LCMS [M+1]⁺: 218.0.

Step B: To a solution of 1-(2-bromo-6-fluorophenyl)-N-methylmethanamine (120 g, 484 mmol, 88% purity, 1.00 eq.) in THF (1.00 L) was added di-tert-butyl dicarbonate (211 g, 968 mmol, 2.00 eq.), and the mixture was stirred at 25° C. for 2 hours. The mixture was then concentrated in vacuo to give a residue. The residue was purified by column chromatography (SiO₂, petroleum ether/ethyl acetate=1/0 to 100/1) to give tert-butyl N-[(2-bromo-6-fluoro-phenyl)methyl]-N-methyl-carbamate (70.0 g, 220 mmol) as a brown oil. LCMS [M−55]⁺: 261.9

¹H NMR (400 MHZ, DMSO-d₆) δ=7.49 (d, J=7.6 Hz, 1H), 7.33-7.26 (m, 2H), 4.57 (s, 2H), 2.64 (s, 3H), 1.38 (s, 9H).

Step C: To a solution of tert-butyl(2-bromo-6-fluorobenzyl)(methyl)carbamate (60.0 g, 189 mmol, 1.00 eq.) and 4,4,4′,4′,5,5,5′,5′-octamethyl-2,2′-bi (1,3,2-dioxaborolane) (60.0 g, 236 mmol, 1.25 eq.) in dioxane (600 mL) was added Pd (dppf) Cl₂·CH₂Cl₂ (15.0 g, 18.4 mmol, 0.10 eq.) and potassium acetate (72.0 g, 734 mmol, 3.89 eq.). The reaction mixture was degassed with nitrogen (3 times) and stirred at 100° C. for 12 hours under a nitrogen atmosphere. The mixture was cooled to 25° C. and concentrated under vacuum to give a residue. The residue was purified by column chromatography (SiO₂, petroleum ether/ethyl acetate=1/0 to 100/1) to give tert-butyl(2-fluoro-6-(4,4,5,5-tetramethyl-1,3,2-dioxaborolan-2-yl)benzyl)(methyl)carbamate (80.0 g, 160 mmol, 73% purity) as a yellow oil. LCMS [M−55]⁺: 266.1.

Step D: To a solution of tert-butyl(2-fluoro-6-(4,4,5,5-tetramethyl-1,3,2-dioxaborolan-2-yl)benzyl)(methyl)carbamate (80.0 g, 160 mmol, 73% purity, 1.00 eq.) and (R)—N-[(1R)-1-(5-bromo-2-thienyl)ethyl]-2-methyl-propane-2-sulfinamide (56.0 g, 180 mmol, 1.13 eq.) in dioxane (500 mL) and water (100 mL) was added cesium carbonate (150 g, 460 mmol, 2.88 eq.) and Pd(PPh₃)₄ (20.0 g, 17.3 mmol, 0.10 eq.) under a nitrogen atmosphere and the mixture was stirred at 100° C. for 3 hours under a nitrogen atmosphere. The mixture was diluted with water (500 mL), extracted with ethyl acetate (1.00 L×2), the organic phase was washed with brine (200 mL), dried over sodium sulfate, filtered and concentrated in vacuo to give a residue. The residue was purified by column chromatography (SiO₂, petroleum ether/ethyl acetate=0/1 to 5/1) to give tert-butyl(2-(5-((R)-1-(((R)-tert-butylsulfinyl)amino)ethyl)thiophen-2-yl)-6-fluorobenzyl)(methyl)carbamate (84.0 g, 152 mmol, 85% purity) as a yellow oil. LCMS [M−100]⁺: 369.1.

¹H NMR (400 MHZ, DMSO-d₆) δ=7.44-7.36 (m, 1H), 7.27-7.17 (m, 2H), 7.08 (br d, J=2.8 Hz, 1H), 6.96 (d, J=3.6 Hz, 1H), 5.88 (br d, J=6.8 Hz, 1H), 4.65 (quin, J=6.4 Hz, 1H), 4.56 (s, 2H), 2.48 (s, 3H), 1.55 (br d, J=6.8 Hz, 3H), 1.33 (br s, 9H), 1.13 (s, 9H).

Step E: To a solution of tert-butyl(2-(5-((R)-1-(((R)-tert-butylsulfinyl)amino)ethyl)thiophen-2-yl)-6-fluorobenzyl)(methyl)carbamate (80.0 g, 145 mmol, 85% purity, 1.00 eq.) in THF (240 mL) and water (48.0 mL) was added iodine (6.80 g, 26.8 mmol, 0.19 eq.). The reaction was heated 50° C. for 2 hours, then diluted with water (500 mL) and extracted with ethyl acetate (500 mL×2). The organic phases were washed with brine (200 mL), dried over sodium sulfate, filtered and concentrated in vacuo to give a residue. The residue was purified by column chromatography (SiO₂, dichloromethane/methanol=300/1 to 10/1) to give tert-butyl (R)-(2-(5-(1-aminoethyl)thiophen-2-yl)-6-fluorobenzyl)(methyl)carbamate (40.0 g, 110 mmol) as yellow oil. LCMS [M−16]⁺: 348.1.

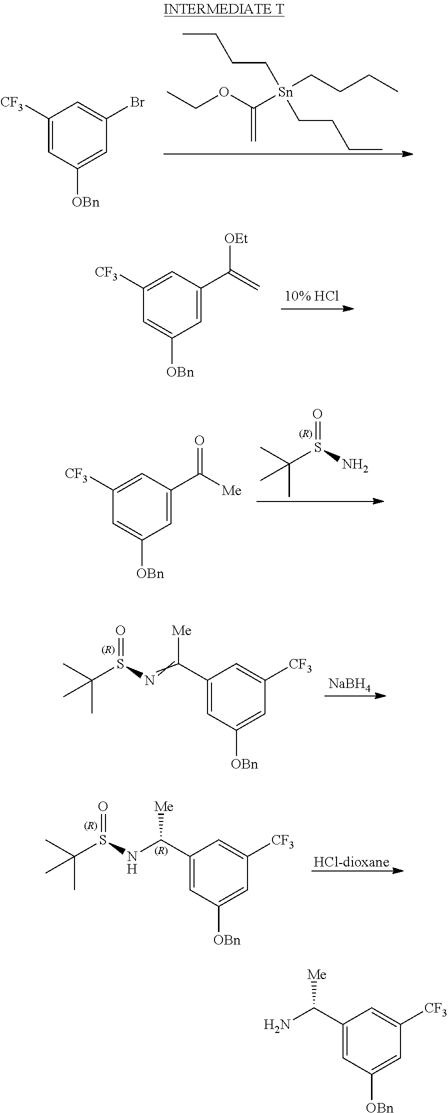

Step A: To a mixture of 1-(benzyloxy)-3-bromo-5-(trifluoromethyl)benzene (3.00 g, 9.06 mmol, 1.00 eq.) and Pd(dppf)Cl₂ (663 mg, 906 μmol, 0.10 eq.) in dioxane (50.0 mL) was added tributyl(1-ethoxyvinyl) tin (5.00 g, 13.8 mmol, 4.67 mL, 1.53 eq.) at 20° C., and the mixture was stirred at 80° C. for 12 hours under a nitrogen atmosphere. To this mixture was then added saturated potassium fluoride solution (100 mL) and the solution was stirred at 20° C. for 1 hour. The mixture was extracted with ethyl acetate (100 mL×3), and the combined organic phases were washed with brine (100 mL×3), dried over anhydrous sodium sulfate, filtered, and concentrated under reduced pressure to give a crude 1-(benzyloxy)-3-(1-ethoxyvinyl)-5-(trifluoromethyl)benzene (2.90 g, crude) as a yellow oil. This crude oil was used in the next step without further purification.

Step B: To a solution of 1-(benzyloxy)-3-(1-ethoxyvinyl)-5-(trifluoromethyl)benzene (2.90 g, 9.00 mmol, crude, 1.00 eq.) in tetrahydrofuran (30.0 mL) was added hydrochloric acid (3.0 M in THF, 10.0 mL, 3.33 eq.), and the solution was stirred at 20° C. for 1 hour. The mixture was then diluted with water (60.0 mL), extracted with ethyl acetate (20.0 mL×3), dried over anhydrous sodium sulfate, filtered, and concentrated under reduced pressure. The residue was purified by column chromatography (silica gel, petroleum ether/ethyl acetate=50/1 to 10/1) to give 1-(3-(benzyloxy)-5-(trifluoromethyl)phenyl)ethan-1-one (2.60 g, 8.84 mmol, 98.2% yield) as a yellow oil.

¹H NMR (400 MHZ, CDCl₃) δ=7.79 (s, 1H), 7.74 (s, 1H), 7.45-7.39 (m, 6H), 5.16 (s, 2H), 2.63 (s, 3H).

Step C: To a solution of 1-(3-(benzyloxy)-5-(trifluoromethyl)phenyl)ethan-1-one (2.60 g, 8.84 mmol, 1.00 eq.) and (R)-2-methylpropane-2-sulfinamide (1.39 g, 11.5 mmol, 1.30 eq.) in tetrahydrofuran (40.0 mL) was added titanium (IV) ethoxide (5.02 g, 17.7 mmol, 5.22 mL, 2.00 eq.) under a nitrogen atmosphere, and the solution was stirred at 70° C. for 12 hours. The mixture was then concentrated under reduced pressure, and the residue was purified by column chromatography (silica gel, petroleum ether/ethyl acetate=20/1 to 10/1) to give (R)—N-(1-(3-(benzyloxy)-5-(trifluoromethyl)phenyl)ethylidene)-2-methylpropane-2-sulfinamide (2.20 g, 5.03 mmol, 57.0% yield) as a yellow oil.

¹H NMR (400 MHZ, CDCl₃) δ=7.45 (d, J=10.0 Hz, 2H), 7.24-7.13 (m, 6H), 4.94 (s, 2H), 2.56 (s, 3H), 1.10 (s, 9H).

Step D: To a mixture of (R)—N-(1-(3-(benzyloxy)-5-(trifluoromethyl)phenyl)ethylidene)-2-methylpropane-2-sulfinamide (2.20 g, 5.54 mmol, 1.00 eq.) in tetrahydrofuran (30.0 mL) was added sodium borohydride (270 mg, 7.14 mmol, 1.29 eq.) at 0° C., and the mixture was stirred at 20° C. for 3 hours. To the mixture was added saturated aqueous ammonium chloride solution (80.0 mL) and the resulting mixture was stirred at 20° C. for 30 minutes. The mixture was then extracted with ethyl acetate (80.0 mL×3), and the combined organic phases were washed with brine (80.0 mL×3), dried over anhydrous sodium sulfate, filtered, and concentrated under reduced pressure. The residue was purified by column chromatography on silica gel (petroleum ether/ethyl acetate=50/1 to 3/1) to give (R)—N—((R)-1-(3-(benzyloxy)-5-(trifluoromethyl)phenyl)ethyl)-2-methylpropane-2-sulfinamide (1.20 g, 3.00 mmol, 54.3% yield) as a yellow oil.

¹H NMR (400 MHZ, CDCl₃) δ=7.53-7.32 (m, 5H), 7.23-7.12 (m, 3H), 5.12 (s, 2H), 4.62-4.53 (m, 1H), 3.43 (d, J=2.8 Hz, 1H), 1.53 (d, J=6.4 Hz, 3H), 1.25 (s, 9H).

Step E: To a solution of (R)—N—((R)-1-(3-(benzyloxy)-5-(trifluoromethyl)phenyl)ethyl)-2-methylpropane-2-sulfinamide (1.20 g, 3.00 mmol, 1.00 eq.) was added hydrochloric acid (4.0 M in dioxane, 751 μL, 1.00 eq.), and the solution was stirred at 20° C. for 20 minutes. The mixture was concentrated under reduced pressure to remove to give (R)-1-(3-(benzyloxy)-5-(trifluoromethyl)phenyl)ethan-1-amine (1.20 g, crude, HCl) as a white solid, which was used without further purification.

¹H NMR (400 MHZ, CDCl₃) δ=8.82 (s, 2H), 7.44-7.31 (m, 8H), 5.09 (s, 2H), 4.42 (s, 1H), 1.43 (s, 3H).

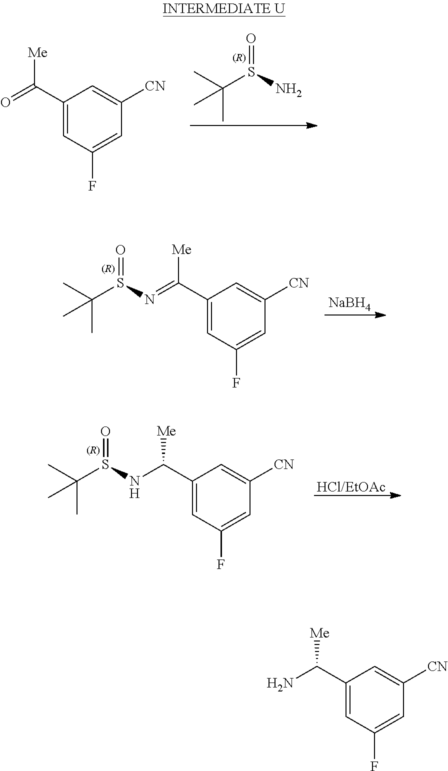

Step A: To a solution of 3-acetyl-5-fluorobenzonitrile (2.00 g, 12.3 mmol, 1.00 eq.) in tetrahydrofuran (20.0 mL) was added titanium ethoxide (5.59 g, 24.5 mmol, 5.08 mL, 2.00 eq.) and (R)-2-methylpropane-2-sulfinamide (1.93 g, 15.9 mmol, 1.30 eq.). The mixture was degassed and purged with nitrogen 3 times, then t stirred at 70° C. for 12 hours under a nitrogen atmosphere. The mixture was diluted with water (20.0 mL) and filtered. The filtrate was extracted with ethyl acetate (30.0 mL×3), dried over anhydrous sodium sulfate, filtered, and concentrated under reduced pressure to give a residue. The residue was purified by column chromatography (SiO₂, petroleum ether/ethyl acetate=50/1 to 1/1) to give (R,E)-N-(1-(3-cyano-5-fluorophenyl)ethylidene)-2-methylpropane-2-sulfinamide (1.01 g, 3.68 mmol, 30.0% yield, 97.5% purity) as a yellow oil. LCMS [M+1]⁺: 267.1.

¹H NMR (400 MHZ, CDCl₃) δ=7.93 (s, 1H), 7.82-7.79 (m, 1H), 7.45-7.52 (m, 1H), 2.79 (s, 3H), 1.35 (s, 9H).

Step B: To a solution of (R,E)-N-(1-(3-cyano-5-fluorophenyl)ethylidene)-2-methylpropane-2-sulfinamide (900 mg, 3.38 mmol, 1.00 eq.) in tetrahydrofuran (10.0 mL) was added sodium borohydride (383 mg, 10.1 mmol, 3.00 eq.) at 0° C. Then the mixture was warmed to 20° C. and stirred for 2 hours. The mixture was quenched with saturated ammonium chloride aqueous solution (20.0 mL) at 25° C., extracted with ethyl acetate (20.0 mL×3), dried over anhydrous sodium sulfate, filtered, and concentrated under reduced pressure to give a residue. The residue was purified by column chromatography (SiO₂, petroleum ether/ethyl acetate=20/1 to 0/1) to give (R)—N—((R)-1-(3-cyano-5-fluorophenyl)ethyl)-2-methylpropane-2-sulfinamide (711 mg, 2.52 mmol, 74.5% yield, 95.3% purity) as a yellow oil. LCMS [M+1]⁺: 269.1.

¹H NMR (400 MHZ, CDCl₃) δ=7.46 (t, J=1.2 Hz, 1H), 7.46-7.33 (m, 1H), 7.31-7.29 (m, 1H), 4.60-4.55 (m, 1H), 3.47 (d, J=3.6 Hz, 1H), 1.54 (d, J=6.8 Hz, 3H), 1.25 (s, 9H).

Step C: To a solution of (R)—N—((R)-1-(3-cyano-5-fluorophenyl)ethyl)-2-methylpropane-2-sulfinamide (711 mg, 2.65 mmol, 1.00 eq.) in dioxane (3.00 mL) was added hydrochloric acid in ethyl acetate (4.0 M, 9.94 mL, 15.0 eq.). The mixture was stirred at 20° C. for 2 hours. The mixture was neutralized with saturated sodium bicarbonate solution (10.0 mL), extracted with ethyl acetate (10.0 mL×3), dried over anhydrous sodium sulfate, filtered, and concentrated under reduced pressure to give (R)-3-(1-aminoethyl)-5-fluorobenzonitrile (330 mg, crude) as a yellow oil.

¹H NMR (400 MHZ, CD₃OD) δ=7.72-7.71 (m, 1H), 7.67-7.66 (m, 1H), 7.65-7.62 (m, 1H), 4.59 (q, J=6.8 Hz, 1H), 1.65 (d, J=6.8 Hz, 3H).

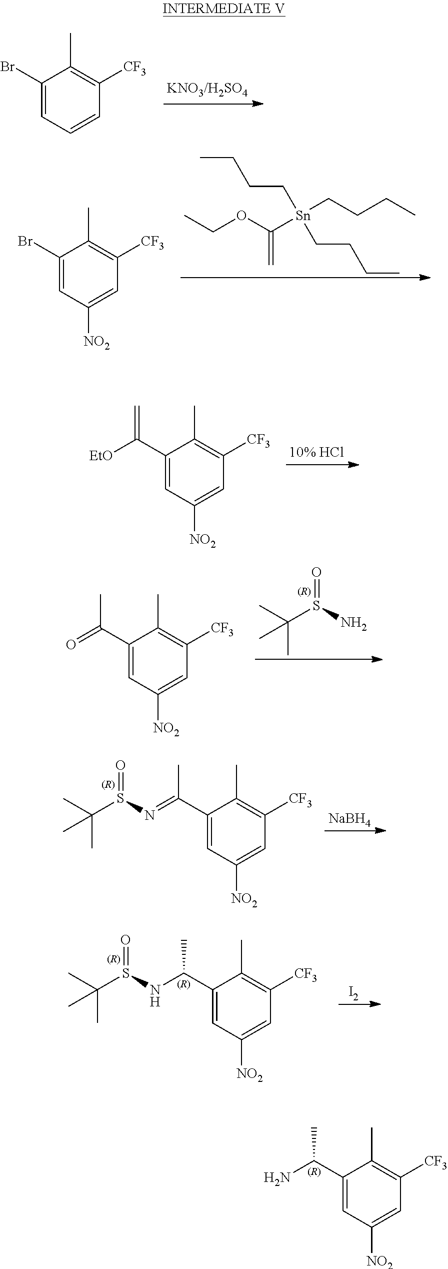

Step A: 1-bromo-2-methyl-3-(trifluoromethyl)benzene (10.0 g, 41.8 mmol, 1.00 eq.) was added the ice-cooled concentrated sulfuric acid (100 mL), then potassium nitrate (12.7 g, 125 mmol, 3.00 eq.) was added slowly at 0° C., then the mixture was stirred at 100° C. for 1 hour. The mixture was then cooled to 25° C., poured into ice-water (500 mL), and extracted with ethyl acetate (300 mL×3). The combined organic layers were washed with brine (400 mL), dried over sodium sulfate, filtered, and concentrated under reduced pressure to give a residue. The residue was purified by column chromatography (SiO₂, petroleum ether/ethyl acetate=1/0 to 1/1) to give 1-bromo-2-methyl-5-nitro-3-(trifluoromethyl)benzene (5.20 g, 16.9 mmol, 40.4% yield) as a white oil.

¹H NMR (400 MHZ, DMSO-d₆) δ=8.72 (d, J=2.0 Hz, 1H), 8.40 (d, J=2.4 Hz, 1H), 2.58-2.62 (m, 3H).

Step B: A mixture of 1-bromo-2-methyl-5-nitro-3-(trifluoromethyl)benzene (5.20 g, 18.3 mmol, 1.00 eq.), tributyl(1-ethoxyvinyl) tin (8.60 g, 23.8 mmol, 8.03 mL, 1.30 eq.) and Pd(PPh₃)₂Cl₂ (385 mg, 549 μmol, 0.03 eq.) in dioxane (60.0 mL) was degassed and purged with nitrogen for 3 times, and then the mixture was stirred at 80° C. for 10 hours under a nitrogen atmosphere. The reaction mixture was quenched with saturated potassium fluoride solution (300 mL) and stirred at 25° C. for 2 hours. Then the suspension extracted with ethyl acetate (180 mL×3). The combined organic layers were washed with brine (200 mL×3), dried over sodium sulfate, filtered, and concentrated under reduced pressure to give 1-(1-ethoxyvinyl)-2-methyl-5-nitro-3-(trifluoromethyl)benzene (6.00 g, crude) as black oil.

¹H NMR (400 MHZ, CD₃OD) δ=8.47 (d, J=2.0 Hz, 1H), 8.32 (d, J=2.0 Hz, 1H), 4.58 (d, J=2.8 Hz, 1H), 4.32 (d, J=2.4 Hz, 1H), 4.00-3.95 (m, 2H), 2.56 (d, J=1.2 Hz, 3H), 1.37 (t, J=7.0 Hz, 3H).

Step C: A mixture of 1-(1-ethoxyvinyl)-2-methyl-5-nitro-3-(trifluoromethyl)benzene (6.00 g, 21.8 mmol, 1.00 eq.) and hydrochloric acid (3.0 M, 20.7 mL, 2.85 eq.) in THF (80.0 mL) was stirred at 20° C. for 1 hour under a nitrogen atmosphere. The reaction mixture was quenched by addition water (100 mL), and then extracted with ethyl acetate (60.0 mL×3). The combined organic layers were washed with brine (70.0 mL), dried over sodium sulfate, filtered, and concentrated under reduced pressure to give a residue. The residue was purified by column chromatography (SiO₂, petroleum ether/ethyl acetate=1/0 to 10/1) to give 1-(2-methyl-5-nitro-3-(trifluoromethyl)phenyl)ethan-1-one (4.10 g, 16.5 mmol, 76.0% yield) as yellow oil.

¹H NMR (400 MHz, CD₃OD) δ=8.67 (s, 1H), 8.57 (s, 1H), 2.66 (s, 3H), 2.60 (s, 3H).

Step D: To a solution of 1-(2-methyl-5-nitro-3-(trifluoromethyl)phenyl)ethan-1-one (2.00 g, 8.09 mmol, 1.00 eq.) and (R)-2-methylpropane-2-sulfinamide (1.27 g, 10.5 mmol, 1.30 eq.) in THF (20.0 mL) was added Ti(OEt)₄ (3.69 g, 16.1 mmol, 3.36 mL, 2.00 eq.), the mixture was stirred at 70° C. for 12 hours under a nitrogen atmosphere. The reaction mixture was diluted with water (70.0 mL) and ethyl acetate (60.0 mL), filtered, and the filtrate was extracted with ethyl acetate (50.0 mL×3). The combined organic layers were dried over sodium sulfate, filtered, and concentrated under reduced pressure to give a residue. The residue was purified by column chromatography (SiO₂, petroleum ether/ethyl acetate=1/0 to 5/1) to give (R,E)-2-methyl-N-(1-(2-methyl-5-nitro-3-(trifluoromethyl)phenyl)ethylidene) propane-2-sulfinamide (2.00 g, 5.71 mmol, 70.5% yield) as yellow oil.

¹H NMR (400 MHZ, CD₃OD) δ=8.43 (s, 1H), 8.30 (s, 1H), 2.75 (s, 3H), 2.58 (s, 3H), 1.30 (m, 9H).

Step E: To a solution of (R,E)-2-methyl-N-(1-(2-methyl-5-nitro-3-(trifluoromethyl)phenyl)ethylidene) propane-2-sulfinamide (2.00 g, 5.71 mmol, 1.00 eq.) in THF (23.0 mL) was added sodium borohydride (647 mg, 17.1 mmol, 3.00 eq.) at 0° C. The mixture was then stirred at 20° C. for 2 hours, and saturated sodium bicarbonate was added, then diluted with water (100 mL). The mixture was extracted with ethyl acetate (60.0 mL×3), the combined organic layers were dried over sodium sulfate, filtered, and concentrated under reduced pressure to give a residue. The residue was purified by column chromatography (SiO₂, petroleum ether/ethyl acetate=1/0 to 0/1) to give (R)-2-methyl-N—((R)-1-(2-methyl-5-nitro-3-(trifluoromethyl)phenyl)ethyl)propane-2-sulfinamide (700 mg, 1.75 mmol, 30.6% yield) as black brown oil. LCMS [M+1]⁺: 353.0.

¹H NMR (400 MHZ, DMSO-d₆) δ=8.67 (d, J=2.4 Hz, 1H), 8.31 (d, J=2.0 Hz, 1H), 6.09 (d, J=7.2 Hz, 1H), 4.83-4.79 (m, 1H), 2.54 (s, 3H), 1.43 (d, J=6.8 Hz, 1H), 1.11 (m, 9H).

Step F: A mixture of (R)-2-methyl-N—((R)-1-(2-methyl-5-nitro-3-(trifluoromethyl)phenyl)ethyl)propane-2-sulfinamide (700 mg, 1.99 mmol, 1.00 eq.) and iodine (151 mg, 595 μmol, 120 μL, 0.30 eq.) in tetrahydrofuran (8.00 mL) and water (2.00 mL) was degassed and purged with nitrogen 3 times, and then the mixture was stirred at 50° C. for 2 hour under nitrogen atmosphere. The reaction was quenched saturated sodium bicarbonate (50.0 mL) and then extracted with ethyl acetate (30.0 mL×3). The combined organic phases were washed with brine (20.0 mL), dried over anhydrous sodium sulfate, filtered, and concentrated under reduced pressure to give a residue. The residue was purified by column chromatography (SiO₂, petroleum ether/ethyl acetate=10/1 to 0/1) to give (R)-1-(2-methyl-5-nitro-3-(trifluoromethyl)phenyl)ethan-1-amine (250 mg, 1.01 mmol, 50.7% yield) as a yellow solid.

¹H NMR (400 MHZ, DMSO-d₆) δ=8.76 (d, J=2.4 Hz, 1H), 8.30 (d, J=2.4 Hz, 1H), 4.54-4.49 (m, 1H), 2.57 (s, 3H), 1.46 (d, J=6.4 Hz, 1H).

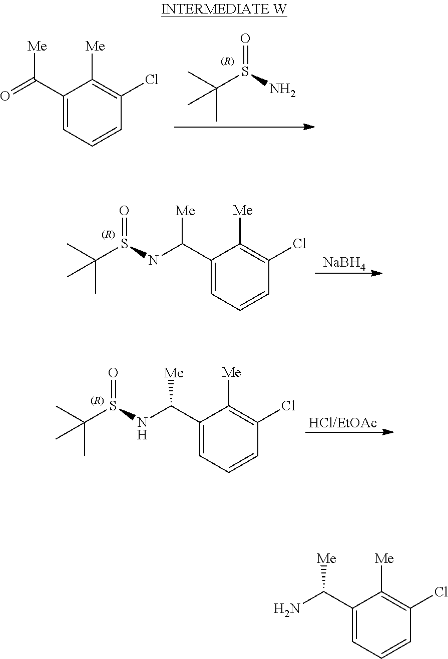

Step A: To a solution of 1-(3-chloro-2-methylphenyl)ethan-1-one (1.50 g, 8.90 mmol, 1.00 eq.) in tetrahydrofuran (30.0 mL) was added titanium ethoxide (6.09 g, 26.7 mmol, 5.53 mL, 3.00 eq.) and (R)-2-methylpropane-2-sulfinamide (1.40 g, 11.6 mmol, 1.30 eq.). The mixture was stirred at 70° C. for 10 hours. The reaction mixture was quenched by sodium bicarbonate (50.0 mL) at 20° C., and then stirred for 10 minutes. The solid was filtered, and the filtrate was extracted with ethyl acetate (20.0 mL×3). The combined organic layers were washed with brine, dried over anhydrous sodium sulfate, filtered, and concentrated under reduced pressure to give (R,E)-N-(1-(3-chloro-2-methylphenyl)ethylidene)-2-methylpropane-2-sulfinamide (2.40 g, crude) as a yellow oil. LCMS [M+1]⁺: 272.0.

Step B: To a solution of (R,E)-N-(1-(3-chloro-2-methylphenyl)ethylidene)-2-methylpropane-2-sulfinamide (2.30 g, 8.46 mmol, 1.00 eq.) in tetrahydrofuran (30.0 mL) was added sodium borohydride (850 mg, 22.5 mmol, 2.66 eq.) at −40° C., the mixture was stirred at −40° C. for 2 hours. The reaction mixture was quenched with saturated ammonium chloride solution (50.0 mL) at 20° C., and then stirred for 10 mins. The solid was filtered off, the filtration was extracted with ethyl acetate (20.0 mL×3). The combined organic layers were washed with brine, dried over anhydrous sodium sulfate, filtered, and concentrated under reduced pressure to give a residue. The residue was purified by column chromatography (SiO₂, petroleum ether/ethyl acetate=1/0 to 1/1) to give (R)—N—((R)-1-(3-chloro-2-methylphenyl)ethyl)-2-methylpropane-2-sulfinamide (1.50 g, 5.48 mmol, 64.7% yield) as a colourless oil. LCMS [M+1]⁻: 274.1.

Step C: To a solution of (R)—N—((R)-1-(3-chloro-2-methylphenyl)ethyl)-2-methylpropane-2-sulfinamide (1.10 g, 4.02 mmol, 1.00 eq.) in ethyl acetate (20.0 mL) was added hydrochloride in ethyl acetate (4.0 M, 30.0 mL) at 0° C., the mixture was stirred at 20° C. for 2 hours. The reaction mixture was concentrated under reduced pressure to give (R)-1-(3-chloro-2-methylphenyl)ethan-1-amine (700 mg, crude) as a white solid. LCMS [M+1]⁺: 170.1.

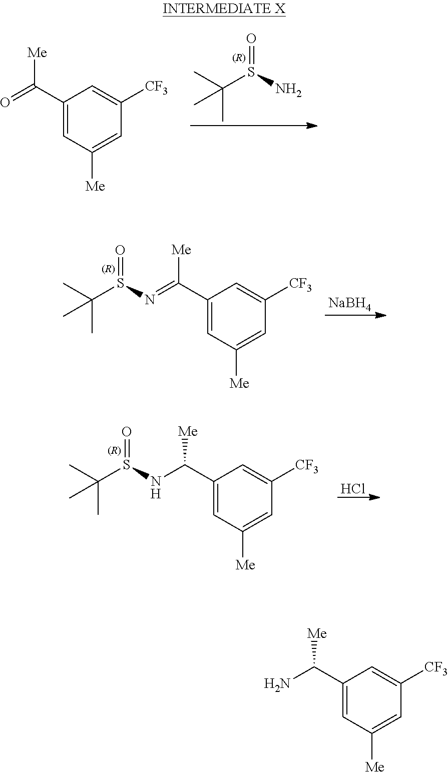

Step A: To a solution of 1-(3-methyl-5-(trifluoromethyl)phenyl)ethan-1-one (500 mg, 2.47 mmol, 1.00 eq.) and (R)-2-methylpropane-2-sulfinamide (689 mg, 5.69 mmol, 2.30 eq.) in THF (7.00 mL) was added Ti(OEt)₄ (1.30 g, 5.69 mmol, 1.18 mL, 2.30 eq.), the mixture was stirred at 70° C. for 12 hours under a nitrogen atmosphere. The reaction mixture was diluted with water (30.0 mL) and ethyl acetate (20.0 mL), filtered and the filtrate was extracted with ethyl acetate (3× 20.0 mL). The combined organic layers were dried over sodium sulfate, filtered, and concentrated under reduced pressure to give a residue. The residue was purified by column chromatography (SiO₂, petroleum ether/ethyl acetate=10/1) to give (R,E)-2-methyl-N-(1-(3-methyl-5-(trifluoromethyl)phenyl)ethylidene) propane-2-sulfinamide (750 mg, 2.46 mmol, 99.3% yield) as a yellow oil. LCMS [M+1]⁺: 306.1.

¹H NMR (400 MHZ, DMSO-d₆) δ=7.99 (s, 1H), 7.95 (s, 1H), 7.75 (s, 1H), 5.75 (s, 1H), 2.76 (s, 3H), 2.46 (s, 3H), 1.22 (s, 9H).

Step B: To a solution of (R,E)-2-methyl-N-(1-(3-methyl-5-(trifluoromethyl)phenyl)ethylidene) propane-2-sulfinamide (650 mg, 2.13 mmol, 1.00 eq.) in THF (15.0 mL) was added sodium borohydride (253 mg, 6.69 mmol, 3.14 eq.) at −40° C. The mixture was stirred at −40° C. for 2 hours. The mixture was added saturated sodium bicarbonate solution and diluted by water (50.0 mL). The mixture was extracted with ethyl acetate (3×50.0 mL), the combined organic layers were dried over sodium sulfate, filtered, and concentrated under reduced pressure to give a residue. The residue was purified by column chromatography (SiO₂, petroleum ether/ethyl acetate=5/1 to 2/1) to give (R)-2-methyl-N—((R)-1-(3-methyl-5-(trifluoromethyl)phenyl)ethyl)propane-2-sulfinamide (320 mg, 1.04 mmol, 48.9% yield) as a light yellow solid. LCMS [M+1]⁺: 308.1.

¹H NMR (400 MHZ, CD₃OD) δ=7.52 (s, 1H), 7.50 (s, 1H), 7.39 (s, 1H), 4.56-4.51 (m, 1H), 2.44 (s, 1H), 1.54-1.53 (d, 3H), 1.25 (s, 9H).

Step C: A solution of (R)-2-methyl-N—((R)-1-(3-methyl-5-(trifluoromethyl)phenyl)ethyl)propane-2-sulfinamide (305 mg, 992 μmol, 1.00 eq.) in hydrochloric acid (4.0 M in ethyl acetate, 10.0 mL), resulting mixture was stirred at 25° C. for 1 hr. Concentrated under reduced pressure to give (R)-1-(3-methyl-5-(trifluoromethyl)phenyl)ethan-1-amine (200 mg, crude) as a light yellow solid. The crude was used directly into next step without further purification. LCMS [M+1]⁺: 204.0.

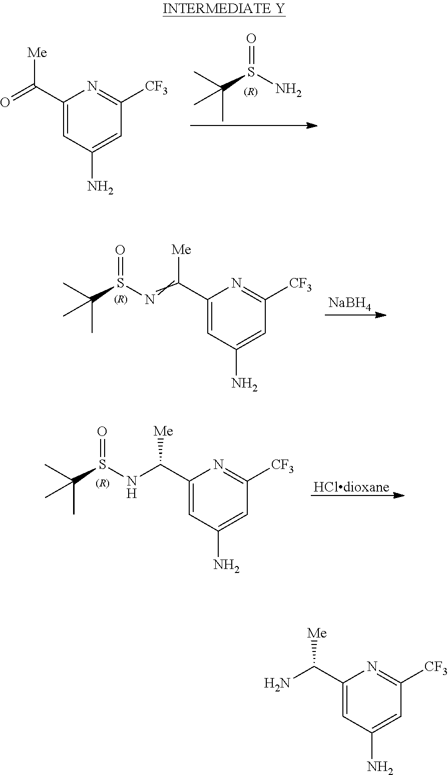

Step A: To a solution of 1-(4-amino-6-(trifluoromethyl)pyridin-2-yl)ethan-1-one (35.6 g, 175 mmol, 1.00 eq.) and (R)-2-methylpropane-2-sulfinamide (25.4 g, 209 mmol, 1.20 eq.) in THF (350 mL) was added titanium (IV) isopropoxide (149 g, 524 mmol, 155 mL, 3.00 eq.), and 1,2-dimethoxyethane (15.7 g, 175 mmol, 18.1 mL, 1.00 eq.). The reaction mixture was stirred at 80° C. for 12 hours, after which point was added water (50.0 mL) to give a suspension. The suspension was filtered, the filtrate was concentrated under reduced pressure to give a residue, the residue was purified by silica gel chromatography (petroleum ether/ethyl acetate=10/1 to 1/1) to give (R)—N-(1-(4-amino-6-(trifluoromethyl)pyridin-2-yl)ethylidene)-2-methylpropane-2-sulfinamide (44.0 g, 143 mmol, 82.0% yield) as brown oil.

¹H NMR (400 MHZ, CDCl₃) δ=7.45 (d, J=2.0 Hz, 1H), 6.97 (d, J=2.0 Hz, 1H), 4.56 (br s, 2H), 2.82 (s, 3H), 1.33 (s, 9H).

Step B: To a solution of (R)—N-(1-(4-amino-6-(trifluoromethyl)pyridin-2-yl)ethylidene)-2-methylpropane-2-sulfinamide (44.0 g, 143 mmol, 1.00 eq.) in THF (400 mL) was added sodium borohydride (16.3 g, 430 mmol, 3.00 eq.) at 0° C. in portionwise, then the reaction was stirred at 0° C. for 1 hour. The mixture was slowly poured into water (200 mL) and stirred for 5 minutes, then extracted with ethyl acetate (300 mL×3). The combined organic phases were washed with brine (200 mL×3), dried with anhydrous sodium sulfate, filtered and concentrated under reduced pressure to give a residue. The residue was purified by column chromatography (SiO₂, petroleum ether/ethyl acetate=10/1 to 1/1) to give (R)—N—((R)-1-(4-amino-6-(trifluoromethyl)pyridin-2-yl)ethyl)-2-methylpropane-2-sulfinamide (24.0 g, 76.2 mmol, 53.2% yield, 98.2% purity) as a brown oil.

¹H NMR (400 MHZ, CDCl₃) δ=6.63 (d, J=2.0 Hz, 1H), 6.56 (d, J=2.0 Hz, 1H), 5.06 (d, J=6.0 Hz, 1H), 4.69 (s, 2H), 4.46-4.39 (m, 1H), 1.45 (d, J=6.8 Hz, 3H), 1.27 (s, 9H).

Step C: To a solution of (R)—N—((R)-1-(4-amino-6-(trifluoromethyl)pyridin-2-yl)ethyl)-2-methylpropane-2-sulfinamide (23.5 g, 76.0 mmol, 1.00 eq) in HCl/dioxane (200 mL) was stirred at 25° C. for 2 hours. The mixture was filtered, and the filter cake was washed with ethyl acetate (100 mL), then the filter cake was collected and dried under vacuum to give (R)-2-(1-aminoethyl)-6-(trifluoromethyl)pyridin-4-amine (hydrochloride salt) as a white solid.

¹H NMR (400 MHZ, DMSO-d₆) δ=8.43 (br s, 3H), 6.93 (br d, J=2.0 Hz, 2H), 6.74 (d, J=1.6 Hz, 1H), 4.34-4.27 (m, 1H), 1.45 (d, J=6.8 Hz, 3H).

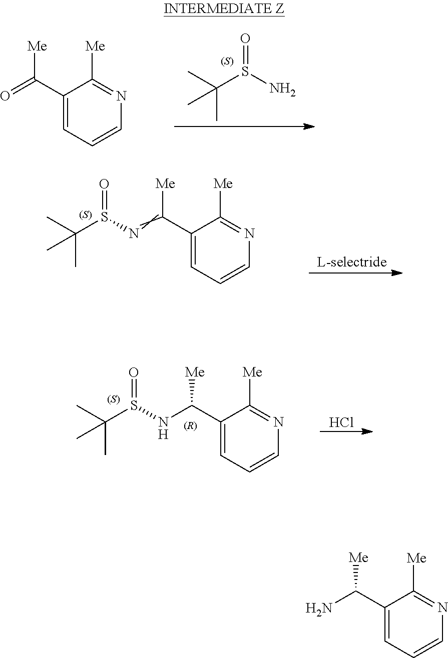

Step A: To a solution of 1-(2-methylpyridin-3-yl)ethan-1-one (800 mg, 5.92 mmol, 1.00 eq.) and (S)-2-methylpropane-2-sulfinamide (933 mg, 7.69 mmol, 1.30 eq.) in tetrahydrofuran (8.00 mL) was added titanium (IV) ethoxide (2.70 g, 11.8 mmol, 2.45 mL, 2.00 eq.) and 1,2-dimethoxyethane (533 mg, 5.92 mmol, 615 μL, 1.00 eq.), and the mixture was stirred at 70° C. for 16 hours. After cooling to 25° C. the mixture was concentrated under reduced pressure and purified by column chromatography (SiO₂, petroleum ether/ethyl acetate=5/1 to 1/1) to give (S)-2-methyl-N-(1-(2-methylpyridin-3-yl)ethylidene) propane-2-sulfinamide (1.25 g, 5.24 mmol, 88.6% yield) as a yellow oil. LCMS [M+1]⁺: 239.2.

Step B: To a solution of (S)-2-methyl-N-(1-(2-methylpyridin-3-yl)ethylidene) propane-2-sulfinamide (1.25 g, 5.24 mmol, 1.00 eq.) in tetrahydrofuran (7.00 mL) was added dropwise L-selectride (1.0 M in THF, 7.87 mL, 1.50 eq.) at −78° C. over 30 minutes, then stirred for an additional 1 hour at −78° C. The reaction mixture was then quenched by addition saturated ammonium chloride solution (in water, 30.0 mL) at 0° C., and stirred for another 1 hour at 25° C. The solution was then extracted with ethyl acetate (50.0 mL×3), and the combined organic layers were washed with brine (30.0 mL×2), dried over anhydrous sodium sulfate, filtered, and concentrated under reduced pressure. The residue was purified twice by column chromatography (SiO₂, petroleum ether/ethyl acetate=5/1 to 0/1) to give (S)-2-methyl-N—((R)-1-(2-methylpyridin-3-yl)ethyl) propane-2-sulfinamide (600 mg, 2.50 mmol, 47.6% yield) as a white solid. LCMS [M+1]⁺: 432.3.

¹H NMR (400 MHZ, CDCl₃) δ=8.36 (dd, J=1.2, 3.6 Hz, 1H), 7.64 (dd, J=1.6, 8.0 Hz, 1H), 7.12 (dd, J=4.8, 7.6 Hz, 1H), 4.81-4.70 (m, 1H), 2.58 (s, 3H), 1.47 (d, J=6.8 Hz, 3H), 1.14 (s, 9H).

SFC conditions: Column: Chiralpak AD-3 50×4.6 mm I.D., 3 μm Mobile phase: Phase A: CO₂, and Phase B: for MeOH (0.05% diethylamine); Gradient elution: MeOH (0.05% diethylamine) in CO₂ from 5% to 40% f Flow rate: 3 mL/min; Detector: PDA Column Temp: 35° C.; Back Pressure: 100 Bar.

Step C: A mixture of (S)-2-methyl-N—((R)-1-(2-methylpyridin-3-yl)ethyl) propane-2-sulfinamide (600 mg, 2.50 mmol, 1.00 eq.) in HCl·dioxane (3.00 mL) was was stirred at 0° C. for 30 minutes under a nitrogen atmosphere. After this time, a white precipitate was formed, and the suspension was filtered. The cake was collected and dried under vacuum, and the residue was further purified by prep-HPLC [column: Waters Xbridge 150×25 mm×5 um; mobile phase: phase A: water (0.05% ammonium hydroxide v/v), phase B: MeCN; B %: 3%-33%] to give (R)-1-(2-methylpyridin-3-yl)ethan-1-amine (370 mg, 2.23 mmol, 89.2% yield, 82% purity) as an colorless oil. LCMS [M−16]⁺: 120.3.

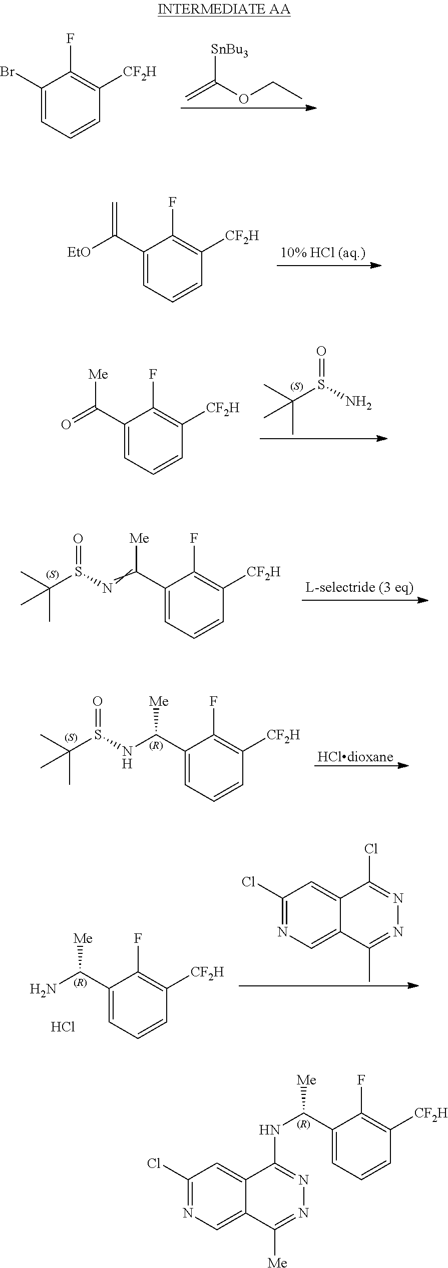

Step A: To a solution of 1-bromo-3-(difluoromethyl)-2-fluorobenzene (commercially available, 4.50 g, 20.0 mmol, 1.00 eq.) in 1,4-dioxane (50.0 mL) was added PdCl₂ (PPh₃) 2 (1.40 g, 2.00 mmol, 0.10 eq.) and tributyl(1-ethoxyvinyl) tin (21.7 g, 60.0 mmol, 20.3 mL, 3.00 eq.), and the mixture was degassed and purged with nitrogen (3 times) then stirred at 100° C. for 3 hours under a nitrogen atmosphere. The mixture was cooled to room temperature, concentrated under reduced pressure, and added potassium fluoride aqueous solution (2.0 M, 100 mL) was added to the residue. The mixture was extracted with ethyl acetate (100 mL×3), dried over anhydrous sodium sulfate, and filtered. The filtrate was concentrated under reduced pressure to give 1-(difluoromethyl)-3-(1-ethoxyvinyl)-2-fluorobenzene (7.50 g, crude) as a brown oil, which was used without further purification.

Step B: To a solution of 1-(difluoromethyl)-3-(1-ethoxyvinyl)-2-fluorobenzene (7.50 g, 34.7 mmol, 1.00 eq.) in tetrahydrofuran (50.0 mL) was added hydrochloric aqueous solution (30.0 mL, 10% purity), and the mixture was stirred at 25° C. for 1 hour. After this time, the pH of the mixture was adjusted to ˜pH to 6-8 with sodium bicarbonate aqueous solution and the mixture was extracted with ethyl acetate (100 mL×3). The combined organic layers were dried over anhydrous sodium sulfate, filtered, and concentrated under reduced pressure. The residue was purified by column chromatography (silica gel, petroleum ether/ethyl acetate=1/0 to 5/1) to give 1-(3-(difluoromethyl)-2-fluorophenyl)ethan-1-one (6.01 g, 31.3 mmol, 90.2% yield, 98.0% purity) as a colorless oil. LCMS [M+1]⁺: 189.1.

¹H NMR (400 MHZ, CDCl₃) δ=8.02-7.97 (m, 1H), 7.80-7.76 (m, 1H), 7.34 (t, J=8.0 Hz, 1H), 6.94 (t, J=14.8 Hz, 1H), 2.66 (d, J=5.2 Hz, 3H).

Step C: A mixture of (S)-2-methylpropane-2-sulfinamide (2.32 g, 19.1 mmol, 1.20 eq.), 1-(3-(difluoromethyl)-2-fluorophenyl)ethan-1-one (3.00 g, 16.0 mmol, 1.00 eq.) and titanium (IV) ethoxide (7.27 g, 31.9 mmol, 6.60 mL, 2.00 eq.) in 2-methyl tetrahydrofuran (30.0 mL) was degassed and purged with nitrogen (3 times), and then stirred at 75° C. for 4 hours under a nitrogen atmosphere. The reaction mixture was then cooled, diluted with water (50.0 mL), extracted with ethyl acetate (50.0 mL×3), and the combined organic layers were washed with brine (100 mL×2), dried over anhydrous sodium sulfate, and filtered. The filtrate was concentrated under reduced pressure, and the residue was purified by column chromatography (silica gel, petroleum ether/ethyl acetate=20/1 to 1/1) to give (S)—N-(1-(3-(difluoromethyl)-2-fluorophenyl)ethylidene)-2-methylpropane-2-sulfinamide (1.80 g, 6.18 mmol, 38.8% yield). LCMS [M+1]⁺: 292.2.

Step D: To a mixture of (S)—N-(1-(3-(difluoromethyl)-2-fluorophenyl)ethylidene)-2-methylpropane-2-sulfinamide (1.80 g, 6.18 mmol, 1.00 eq.) in 2-methyl tetrahydrofuran (30.0 mL) was added L-selectride (3.52 g, 18.5 mmol, 4.10 mL, 3.00 eq.) under a nitrogen atmosphere at −78° C., and then the mixture was stirred at −78° C. for 3 hours under a nitrogen atmosphere. After this time, additional L-selectride (1.76 g, 9.30 mmol, 2.00 mL, 1.50 eq.) was added and the solution was degassed and purged with nitrogen (3 times) and stirred at −78° C. for 9 hours under a nitrogen atmosphere. The mixture was cooled to room temperature, diluted with water (30.0 mL), and extracted with ethyl acetate (30.0 mL×3). The combined organic layers were washed with brine (30.0 mL×2), dried over anhydrous sodium sulfate, filtered, and concentrated under reduced pressure. The residue was purified by column chromatography (silica gel, petroleum ether/ethyl acetate=20/1 to 1/1) to give (S)—N—((R)-1-(3-(difluoromethyl)-2-fluorophenyl)ethyl)-2-methylpropane-2-sulfinamide (1.30 g, 4.34 mmol, 70.3% yield, 98% purity) as a colorless oil. LCMS [M+1]⁺: 294.2.

Step E: To a solution of (S)—N—((R)-1-(3-(difluoromethyl)-2-fluorophenyl)ethyl)-2-methylpropane-2-sulfinamide (1.29 g, 4.43 mmol, 1.00 eq.) was added hydrochloric acid (4.00 M in 1,4-dioxane, 15.0 mL, 14.0 eq.), and the mixture was stirred at 25° C. for 30 minutes. The mixture was then diluted with water (30.0 mL), extracted with ethyl acetate (30.0 mL×3), and the combined organic layers were washed with brine (30.0 mL×2), dried over anhydrous sodium sulfate, and filtered. The filtrate was concentrated under reduced pressure to (R)-1-(3-(difluoromethyl)-2-fluorophenyl)ethan-1-amine (480 mg, 2.13 mmol, 48.0% yield, HCl salt) as a yellow oil, which was used without further purification.

¹H NMR (400 MHZ, CDCl₃) δ=7.52-7.47 (m, 2H), 7.24-7.19 (m, 1H), 6.88 (t, J=14.8 Hz, 1H), 4.85-4.92 (m, 1H), 1.57 (d, J=6.8 Hz, 3H).

Step F: A mixture of (R)-1-(3-(difluoromethyl)-2-fluorophenyl)ethan-1-amine (300 mg, 1.59 mmol, 1.00 eq.), 1,7-dichloro-4-methylpyrido[3,4-d]pyridazine (339 mg, 1.59 mmol, 1.00 eq.) and potassium fluoride (461 mg, 7.93 mmol, 186 μL, 5.00 eq.) in dimethyl sulfoxide (6.00 mL) was degassed and purged with nitrogen (3 times), and the mixture was stirred at 130° C. for 12 hours under a nitrogen atmosphere. The mixture was then cooled to 25° C., diluted with water (30.0 mL), and extracted with ethyl acetate (30.0 mL×3). The combined organic layers were washed with brine (30.0 mL×3), dried over sodium sulfate, filtered, and concentrated under reduced pressure. The residue was purified by column chromatography (silica gel, petroleum ether/ethyl acetate=10/1 to 1/1) and prep-HPLC [column: Phenomenex luna C18 150×25 mm×10 μm; mobile phase: phase A: water (0.225% formic acid), phase B: acetonitrile; B %: 20%-50%] to give (R)-7-chloro-N-(1-(3-(difluoromethyl)-2-fluorophenyl)ethyl)-4-methylpyrido[3,4-d]pyridazin-1-amine (250 mg, 629 μmol, 39.7% yield, 92.3% purity) as a yellow solid. LCMS [M+1]⁺: 367.2.

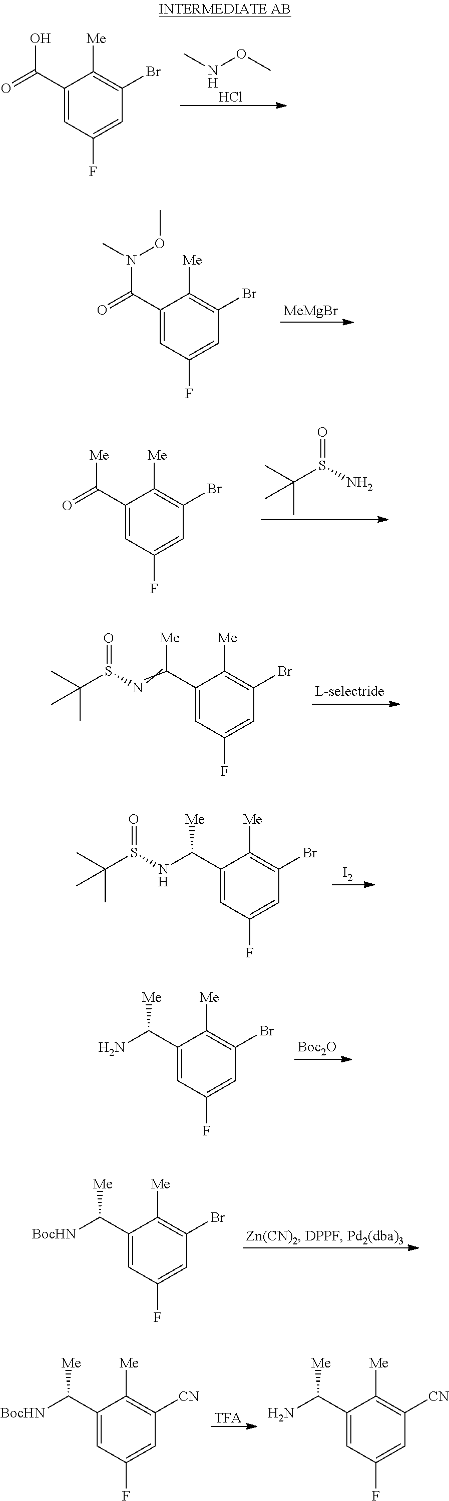

Step A: To a solution of 3-bromo-5-fluoro-2-methylbenzoic acid (4.00 g, 17.2 mmol, 1.00 eq.) and N,O-dimethylhydroxylamine (1.84 g, 18.9 mmol, 1.10 eq., HCl salt) in DMF (50.0 mL) was added N,N-diisopropylethylamine (6.66 g, 51.5 mmol, 8.97 mL, 3.00 eq.) and HATU (7.83 g, 20.6 mmol, 1.20 eq.), and the reaction mixture was stirred at 20° C. for 2 hours. The reaction mixture was diluted with ethyl acetate (50.0 mL), washed with brine (30.0 mL×3), and the combined organic phases were collected, dried over sodium sulfate, filtered, and concentrated under reduced pressure. The residue was purified by column chromatography (SiO₂, petroleum ether/ethyl acetate=5/1 to 2/1) to give 3-bromo-5-fluoro-N-methoxy-N,2-dimethylbenzamide (4.70 g, 17.0 mmol, 99.2% yield) as a white solid.

Step B: To a solution of 3-bromo-5-fluoro-N-methoxy-N,2-dimethyl-benzamide (4.70 g, 17.0 mmol, 1.00 eq.) in THF (100 mL) was added methylmagnesium bromide (3.0 M, 34.1 mL, 6.00 eq.) dropwise at 0° C. After dropwise addition was completed, the reaction mixture was warmed to 45° C. and stirred for 5 hours. The mixture was then cooled to 25° C., quenched by water (20.0 mL), and extracted with ethyl acetate (50.0 mL×3). The combined organic phases were dried over sodium sulfate, filtered, and concentrated under reduced pressure. The residue was purified by column chromatography (SiO₂, petroleum ether/ethyl acetate=5/1) to give 1-(3-bromo-5-fluoro-2-methylphenyl)ethan-1-one (3.80 g, 16.5 mmol, 96.6% yield) as a light yellow solid.

¹H NMR (400 MHZ, CDCl₃) δ=7.43 (dd, J=2.8, 7.6 Hz, 1H), 7.19 (dd, J=2.8, 8.4 Hz, 1H), 2.55 (s, 3H), 2.45 (d, J=0.4 Hz, 3H).

Step C: To a solution of 1-(3-bromo-5-fluoro-2-methylphenyl)ethan-1-one (3.80 g, 16.5 mmol, 1.00 eq.) and (S)-2-methylpropane-2-sulfinamide (2.79 g, 23.0 mmol, 1.40 eq.) in THF (60.0 mL) was added titanium (IV) ethoxide (7.50 g, 32.9 mmol, 6.82 mL, 2.00 eq.) and 1,2-dimethoxyethane (1.48 g, 16.5 mmol, 1.71 mL, 1.00 eq.), and the mixture was stirred at 70° C. for 12 hours. The reaction mixture was then cooled to 25° C., diluted with ethyl acetate (100 mL) and water (10.0 mL) to give a suspension. The suspension was filtered, and the filtrate was concentrated under reduced pressure to remove all volatiles. The residue was purified by column chromatography (SiO₂, petroleum ether/ethyl acetate=30/1 to 20/1) to give (S)—N-(1-(3-bromo-5-fluoro-2-methylphenyl)ethylidene)-2-methylpropane-2-sulfinamide (4.70 g, 14.1 mmol, 85.5% yield) as yellow oil. LCMS [M+3]⁺: 336.0.

¹H NMR (400 MHZ, CDCl₃) δ=7.35 (br dd, J=2.4, 7.6 Hz, 1H), 6.92 (dd, J=2.4, 8.4 Hz, 1H), 2.66 (s, 3H), 2.37 (s, 3H), 1.30 (s, 9H).

Step D: To a solution of (S)—N-(1-(3-bromo-5-fluoro-2-methylphenyl)ethylidene)-2-methylpropane-2-sulfinamide (5.50 g, 16.5 mmol, 1.00 eq.) in THF (80.0 mL) was added L-selectride (1.0 M, 24.7 mL, 1.50 eq.) dropwise at −78° C., and the reaction mixture was warmed to 0° C. and stirred for 2 hours. The mixture was then diluted with ammonium chloride aqueous solution (30.0 mL), and the resulting solution was extracted with ethyl acetate (50.0 mL×2). The combined organic phases were dried over sodium sulfate, filtered, and concentrated under reduced pressure. The residue was triturated with petroleum ether (20.0 mL), filtered, and the filter cake was dried under vacuum to give (S)—N—((R)-1-(3-bromo-5-fluoro-2-methylphenyl)ethyl)-2-methylpropane-2-sulfinamide (3.20 g, 9.52 mmol, 57.8% yield) as a white solid.

¹H NMR (400 MHZ, CDCl₃) δ=7.24 (dd, J=2.4, 7.6 Hz, 1H), 7.10 (dd, J=2.8, 10.0 Hz, 1H), 4.90-4.82 (m, 1H), 3.30 (br d, J=2.8 Hz, 1H), 2.42 (s, 3H), 1.48 (d, J=6.8 Hz, 3H), 1.23 (s, 9H).

Step E: To a solution of (S)—N—((R)-1-(3-bromo-5-fluoro-2-methylphenyl)ethyl)-2-methylpropane-2-sulfinamide (1.60 g, 4.76 mmol, 1.00 eq.) in THF (20.0 mL) and water (5.00 mL) was added iodine (362 mg, 1.43 mmol, 288 μL, 0.30 eq.), and the mixture was stirred at 50° C. for 2 hours. The mixture was then cooled to 25° C., and the pH was adjusted to pH=7 with sodium bicarbonate aqueous solution. The resulting solution was extracted with DCM (20.0 mL×3), and the combined organic phases were dried over sodium sulfate, filtered, and concentrated under reduced pressure to give (R)-1-(3-bromo-5-fluoro-2-methylphenyl)ethan-1-amine (1.20 g, crude) as a light yellow oil. This crude oil was used without further purification.

Step F: To a solution of (R)-1-(3-bromo-5-fluoro-2-methylphenyl)ethan-1-amine (1.20 g, 5.17 mmol, 1.00 eq.) in THF (20.0 mL) was added di-tert-butyl dicarbonate (1.35 g, 6.20 mmol, 1.43 mL, 1.20 eq.), and the mixture was stirred at 20° C. for 3 hours. The mixture was then concentrated under reduced pressure, and the residue was purified by column chromatography (SiO₂, petroleum ether/ethyl acetate=150/1 to 70/1) to give tert-butyl (R)-(1-(3-bromo-5-fluoro-2-methylphenyl)ethyl)carbamate (1.45 g, 4.36 mmol, 84.4% yield) as a white solid.

Step G: A mixture of tert-butyl (R)-(1-(3-bromo-5-fluoro-2-methylphenyl)ethyl)carbamate (1.35 g, 4.06 mmol, 1.00 eq.), zinc cyanide (954 mg, 8.13 mmol, 516 μL, 2.00 eq.), DPPF (451 mg, 813 μmol, 0.20 eq.), zinc powder (26.6 mg, 406 μmol, 0.10 eq.) and Pd₂(dba)₃ (372 mg, 406 μmol, 0.10 eq.) in dimethylacetamide (20.0 mL) was degassed and purged with nitrogen (3 times), and the mixture was stirred at 120° C. for 6 hours under a nitrogen atmosphere. The mixture was then diluted with ethyl acetate (60.0 mL), filtered, and the filtrate was washed with brine (30.0 mL×3), dried over sodium sulfate, and concentrated under reduced pressure. The residue was purified by column chromatography (SiO₂, petroleum ether/ethyl acetate=100/1 to 30/1) to give tert-butyl (R)-(1-(3-cyano-5-fluoro-2-methylphenyl)ethyl)carbamate (1.10 g, 3.95 mmol, 97.3% yield) as a light yellow solid.

Step H: To a solution of tert-butyl (R)-(1-(3-cyano-5-fluoro-2-methylphenyl)ethyl)carbamate (1.10 g, 3.95 mmol, 1.00 eq.) in DCM (5.00 mL) was added TFA (1.88 g, 16.5 mmol, 1.22 mL, 4.18 eq.), and the mixture was stirred at 20° C. for 1 hour. The mixture was then concentrated under reduced pressure, and the residue was adjusted to pH=7 with saturated sodium bicarbonate aqueous solution. The resulting solution was extracted with DCM (50.0 mL), and the organic phase was dried over sodium sulfate, and concentrated in vacuum to give (R)-3-(1-aminoethyl)-5-fluoro-2-methylbenzonitrile (0.80 g, crude) as brown oil which was used without further purification.

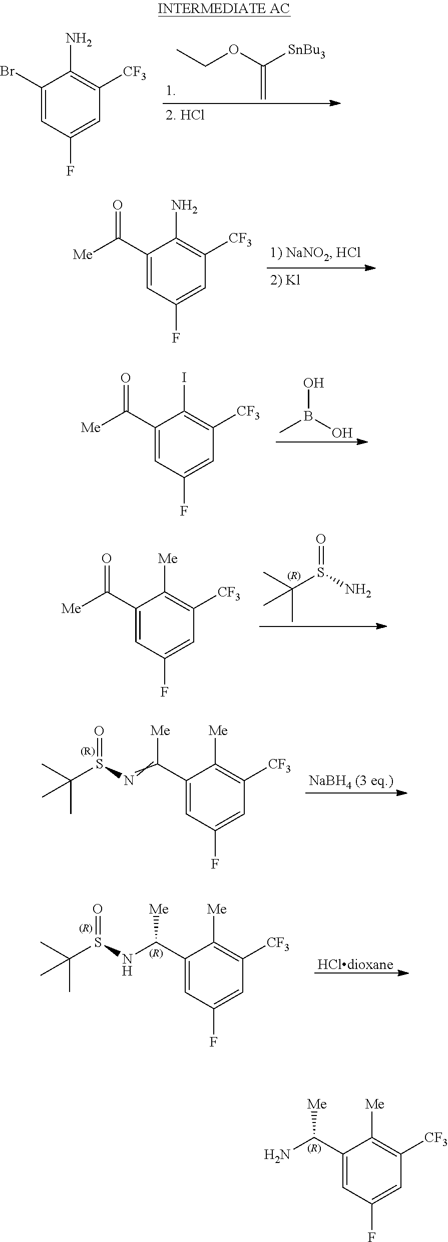

Step A: To a solution of 2-bromo-4-fluoro-6-(trifluoromethyl) aniline (2.00 g, 7.75 mmol, 1.00 eq.) and tributyl(1-ethoxyvinyl) tin (2.80 g, 7.75 mmol, 2.62 mL, 1.00 eq.) in dioxane (20.0 mL) was added PdCl₂ (PPh₃) 2 (544 mg, 775 μmol, 0.10 eq.) under a nitrogen atmosphere, and the mixture was stirred at 80° C. for 12 hours. The reaction mixture was then cooled to 25° C., diluted with potassium fluoride aqueous solution (100 mL) and then extracted with ethyl acetate (100 mL×3). The combined organic layers were washed with brine (100 mL), dried over sodium sulfate, filtered, and concentrated under reduced pressure to give compound 2-(1-ethoxyvinyl)-4-fluoro-6-(trifluoromethyl) aniline (4.00 g, crude) as a yellow oil. To a solution of 2-(1-ethoxyvinyl)-4-fluoro-6-(trifluoromethyl) aniline (4.00 g, crude) in tetrahydrofuran (50.0 mL) was added hydrochloric acid aqueous solution (4.00 M, 20.0 mL, 1.33 eq.) dropwise. Then the mixture was stirred at 25° C. for 1 hour, diluted with water (100 mL) and extracted with ethyl acetate (300 mL×3). The combined organic layers were washed with brine (200 mL), dried over sodium sulfate, filtered, and concentrated under reduced pressure. The residue was purified by column chromatography (petroleum ether/ethyl acetate=30/1 to 3/1) to give compound 1-(2-amino-5-fluoro-3-(trifluoromethyl)phenyl)ethan-1-one (5.60 g, 25.3 mmol, 42.0% yield, 99.9% purity) as a yellow solid.

¹H NMR (400 MHZ, DMSO-d₆) δ=7.99 (d, J=9.2 Hz, 1H), 7.65-7.61 (m, 1H), 7.33 (s, 2H), 2.59 (s, 3H).

Step B: To a solution of 1-(2-amino-5-fluoro-3-(trifluoromethyl)phenyl)ethan-1-one (5.60 g, 25.3 mmol, 1.00 eq.) in hydrochloric acid (50.0 mL) and water (100 mL) was added sodium nitrite (2.27 g, 32.9 mmol, 1.30 eq.) portionwise, then potassium iodide (8.41 g, 50.6 mmol, 2.00 eq.) was added to the mixture at 0° C. After the addition was finished, the reaction mixture was stirred at 25° C. for 12 hours then diluted with water (100 mL), and extracted with ethyl acetate (200 mL×3). The combined organic layers were washed with sodium sulfite (200 mL×3), dried over sodium sulfate, filtered, and concentrated under reduced pressure. The residue was purified by column chromatography (petroleum ether/ethyl acetate=50/1 to 10/1) to give compound 1-(5-fluoro-2-iodo-3-(trifluoromethyl)phenyl)ethan-1-one (5.60 g, 10.3 mmol, 40.8% yield, 61.2% purity) as a yellow solid.

¹H NMR (400 MHZ, DMSO-d₆) δ=7.83-7.76 (m, 1H), 7.74-7.71 (m, 1H), 2.56 (s, 3H).

Step C: To a solution of methylboronic acid (1.62 g, 27.1 mmol, 2.50 eq.) and 1-(5-fluoro-2-iodo-3-(trifluoromethyl)phenyl)ethan-1-one (3.60 g, 10.8 mmol, 1.00 eq.) in dioxane (20.0 mL) was added Pd(dppf)Cl₂ (400 mg, 542 μmol, 0.05 eq.) and potassium carbonate (7.49 g, 54.2 mmol, 5.00 eq.) under a nitrogen atmosphere, and the mixture was stirred at 90° C. for 12 hours. The mixture was then cooled to 25° C., diluted with water (50.0 mL) and extracted with ethyl acetate (100 mL×3). The combined organic layers were washed with brine (100 mL), dried over sodium sulfate, filtered, and concentrated under reduced pressure. The residue was purified by column chromatography (petroleum ether/ethyl acetate=50/1 to 10/1) to give compound 1-(5-fluoro-2-methyl-3-(trifluoromethyl)phenyl)ethan-1-one (1.70 g, 7.72 mmol, 71.2% yield) as a yellow oil.

¹H NMR (400 MHZ, CDCl₃) δ=7.47 (dd, J=2.8, 8.8 Hz, 1H), 7.36-7.30 (m, 1H), 2.58 (s, 3H), 2.47 (s, 3H).

Step D: To a solution of 1-(5-fluoro-2-methyl-3-(trifluoromethyl)phenyl)ethan-1-one (2.20 g, 9.99 mmol, 1.00 eq.) and (R)-2-methylpropane-2-sulfinamide (2.42 g, 20.0 mmol, 2.00 eq.) in tetrahydrofuran (15.0 mL) was added titanium (IV) isopropoxide (5.68 g, 20.0 mmol, 5.90 mL, 2.00 eq.) and 1-methoxy-2-(2-methoxyethoxy)ethane (4.12 g, 30.7 mmol, 4.40 mL, 3.08 eq), and the mixture was stirred at 75° C. for 12 hours. The mixture was then cooled to 25° C., diluted with water (50.0 mL) to give a suspension. The resulting suspension was filtered, and the filtrate was diluted with ethyl acetate (100 mL×3). The combined organic layers were washed with brine (50.0 mL), dried over sodium sulfate, filtered, and concentrated under reduced pressure. The residue was purified by column chromatography (petroleum ether/ethyl acetate=10/1 to 3/1) to give compound (R)—N-(1-(5-fluoro-2-methyl-3-(trifluoromethyl)phenyl)ethylidene)-2-methylpropane-2-sulfinamide (1.50 g, 4.64 mmol, 46.4% yield) as a yellow oil.

¹H NMR (400 MHZ, CDCl₃) δ=7.39 (dd, J=2.2, 8.8 Hz, 1H), 7.10 (dd, J=2.4, 8.4 Hz, 1H), 2.68 (s, 3H), 2.41 (s, 3H), 1.30 (s, 9H).

Step E: To a solution of (R)—N-(1-(5-fluoro-2-methyl-3-(trifluoromethyl)phenyl)ethylidene)-2-methylpropane-2-sulfinamide (1.90 g, 5.88 mmol, 1.00 eq.) in tetrahydrofuran (20.0 mL) was added sodium borohydride (667 mg, 17.6 mmol, 3.00 eq.) portionwise at 0° C. The reaction mixture was stirred at 0° C. for 2 hours, then diluted slowly with saturated aqueous ammonium chloride (50.0 mL) and stirred for 30 minutes. The resulting mixture was extracted with ethyl acetate (100 mL×3), and the combined organic layers were dried over anhydrous sodium sulfate, filtered, and concentrated under reduced pressure. The residue was purified by column chromatography (petroleum ether/ethyl acetate=10/1 to 3/1) to afford (R)—N—((R)-1-(5-fluoro-2-methyl-3-(trifluoromethyl)phenyl)ethyl)-2-methylpropane-2-sulfinamide (1.30 g, 4.00 mmol, 68.0% yield) as a yellow oil.

¹H NMR (400 MHZ, CDCl₃) δ=7.40-7.28 (m, 2H), 4.95-4.84 (m, 1H), 3.40-3.32 (m, 1H), 2.43 (s, 3H), 1.49 (d, J=6.4 Hz, 3H), 1.23 (s, 9H).

Step F: To a solution of (R)—N—((R)-1-(5-fluoro-2-methyl-3-(trifluoromethyl)phenyl)ethyl)-2-methylpropane-2-sulfinamide (1.30 g, 4.00 mmol, 1.00 eq.) in dichloromethane (5.00 mL) was added hydrochloric acid (4.00 M in 1,4-dioxane, 5.00 mL, 5.0 eq.), and the mixture was stirred at 25° C. for 1 hour. The mixture was then concentrated under reduced pressure to give compound (R)-1-(5-fluoro-2-methyl-3-(trifluoromethyl)phenyl)ethan-1-amine (700 mg, 2.81 mmol, 70.4% yield, 88.9% purity, HCl salt) as a yellow oil, which was used directly without further purification.

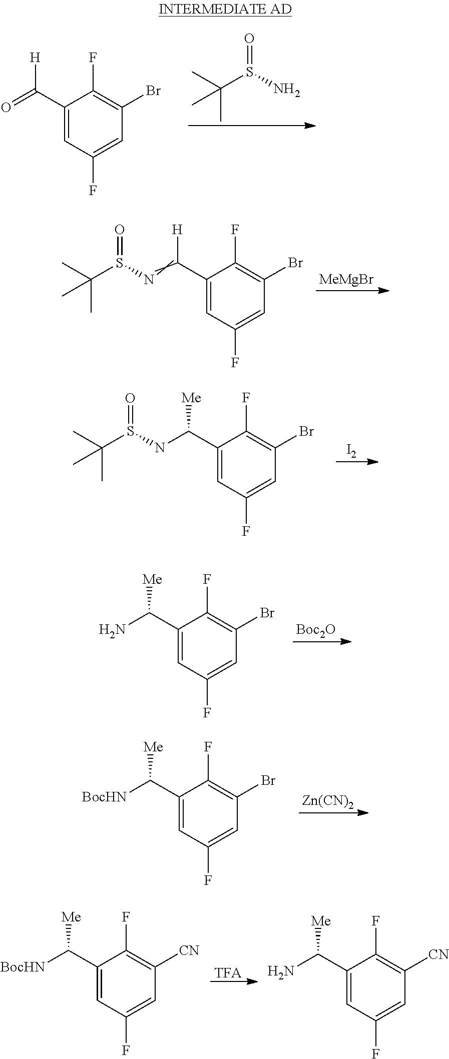

Step A: To a solution of 3-bromo-2,5-difluorobenzaldehyde (4.00 g, 18.1 mmol, 1.00 eq.) and (R)-2-methylpropane-2-sulfinamide (3.07 g, 25.3 mmol, 1.40 eq.) in THF (50.0 mL) was added titanium (IV) ethoxide (8.26 g, 36.2 mmol, 7.51 mL, 2.00 eq.) and 1,2-dimethoxyethane (1.63 g, 18.1 mmol, 1.88 mL, 1.00 eq.), and the mixture was stirred at 70° C. for 12 hours. The mixture was then cooled to 25° C., diluted with ethyl acetate (50.0 mL) and water (5.00 mL) slowly to give a suspension. The suspension was filtered, and the filtrate was concentrated under reduced pressure then purified by column chromatography (SiO₂, petroleum ether/ethyl acetate=20/1 to 10/1) to give (S)—N-(3-bromo-2,5-difluorobenzylidene)-2-methylpropane-2-sulfinamide (5.70 g, 17.6 mmol, 97.1% yield) as a white solid.

¹H NMR (400 MHZ, CDCl₃) δ=8.81 (d, J=2.4 Hz, 1H), 7.74 (dd, J=6.0, 8.4 Hz, 1H), 7.44 (dd, J=5.2, 8.8 Hz, 1H), 1.28 (s, 9H).

Step B: To a solution of (S)—N-(3-bromo-2,5-difluorobenzylidene)-2-methylpropane-2-sulfinamide (5.50 g, 17.0 mmol, 1.00 eq.) in DCM (60.0 mL) was added methylmagnesium bromide (3.0 M, 17.0 mL, 3.00 eq.) dropwise at −60° C., and then the mixture was warmed to 0° C. and stirred for 1 hour. The mixture was diluted with ammonium chloride aqueous solution (50.0 mL), and the resulting aqueous solution was extracted with ethyl acetate (50.0 mL×3). The combined organic phases were dried over sodium sulfate, filtered, and concentrated under reduced pressure. The residue was purified by column chromatography (SiO₂, petroleum ether/ethyl acetate=5/1 to 2/1) to give (S)—N—((R)-1-(3-bromo-2,5-difluorophenyl)ethyl)-2-methylpropane-2-sulfinamide (3.50 g, 10.3 mmol, 60.6% yield) as a white solid.

¹H NMR (400 MHZ, CDCl₃) δ=7.31-7.26 (m, 1H), 7.16 (dd, J=6.4, 8.8 Hz, 1H), 4.89-4.78 (m, 1H), 3.35 (br d, J=4.0 Hz, 1H), 1.56 (d, J=6.8 Hz, 3H), 1.23 (s, 9H).

Step C: To a solution of (S)—N—((R)-1-(3-bromo-2,5-difluorophenyl)ethyl)-2-methylpropane-2-sulfinamide (1.50 g, 4.41 mmol, 1.00 eq.) in THF (20.0 mL) and water (5.00 mL) was added iodine (336 mg, 1.32 mmol, 266 μL, 0.30 eq.), and the mixture was stirred at 50° C. for 2 hours. The mixture was then cooled to 25° C., and the pH was adjusted to pH=7 with sodium bicarbonate aqueous solution. The resulting aqueous solution was extracted with DCM (20.0 mL×3), and the combined organic phases were dried over sodium sulfate, filtered, and concentrated under reduced pressure to give (R)-1-(3-bromo-2,5-difluorophenyl)ethan-1-amine (1.20 g, crude) as a light yellow oil. This crude oil was used directly without further purification.

Step D: To a solution of (R)-1-(3-bromo-2,5-difluorophenyl)ethan-1-amine (1.20 g, 5.08 mmol, 1.00 eq.) in THF (20.0 mL) was added di-tert-butyl dicarbonate (1.22 g, 5.59 mmol, 1.28 mL, 1.10 eq.), and the mixture was stirred at 20° C. for 2 hours. The reaction mixture was concentrated under reduced pressure, and purified by column chromatography (SiO₂, petroleum ether/ethyl acetate=150/1 to 80/1) to give tert-butyl (R)-(1-(3-bromo-2,5-difluorophenyl)ethyl)carbamate (1.30 g, 3.87 mmol, 76.1% yield) as a white solid.

Step E: A mixture of tert-butyl (R)-(1-(3-bromo-2,5-difluorophenyl)ethyl)carbamate (1.20 g, 3.57 mmol, 1.00 eq.), zinc cyanide (838 mg, 7.14 mmol, 453 μL, 2.00 eq.), zinc (23.3 mg, 357 μmol, 0.10 eq.), DPPF (396 mg, 714 μmol, 0.20 eq.) and Pd₂(dba)₃ (327 mg, 357 μmol, 0.10 eq.) in dimethylacetamide (20.0 mL) was degassed and purged with nitrogen (3 times), and the mixture was stirred at 115° C. for 3 hours under a nitrogen atmosphere. The mixture was then cooled 25° C., diluted with ethyl acetate (100 mL), and the organic phase was washed with brine (50.0 mL×3), dried over sodium sulfate, filtered, and concentrated under reduced pressure. The residue was purified by column chromatography (SiO₂, petroleum ether/ethyl acetate=100/1 to 30/1) to give tert-butyl (R)-(1-(3-cyano-2,5-difluorophenyl)ethyl)carbamate (0.90 g, 3.19 mmol, 89.3% yield) as a light yellow solid.

Step F: To a solution of tert-butyl (R)-(1-(3-cyano-2,5-difluorophenyl)ethyl)carbamate (0.90 g, 3.19 mmol, 1.00 eq.) in DCM (10.0 mL) was added TFA (4.62 g, 40.5 mmol, 3.00 mL, 12.7 eq.), and the reaction mixture was stirred at 20° C. for 1 hour. The reaction mixture was then concentrated under reduced pressure, and the residue was diluted with water (10.0 mL). The pH of the solution was adjusted to pH=7 with sodium bicarbonate aqueous solution, and the resulting aqueous solution was extracted with DCM (20.0 mL×2). The combined organic phases were dried over sodium sulfate, filtered, and concentrated under reduced pressure to give (R)-3-(1-aminoethyl)-2,5-difluorobenzonitrile (700 mg, crude) as light-yellow oil. This compound was used directly without further purification.

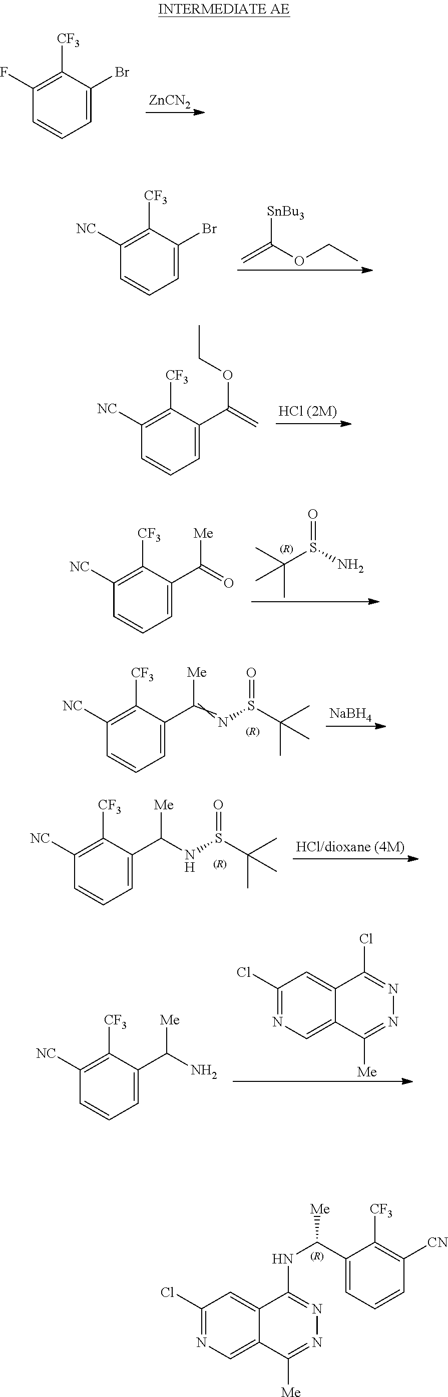

Step A: To a solution of 1-bromo-3-fluoro-2-(trifluoromethyl)benzene (39.0 g, 160 mmol, 1.00 eq.) in dimethylsulfoxide (200 mL) was added zinc cyanide (11.5 g, 176 mmol, 7.56 mL, 1.10 eq.), and the reaction mixture was stirred at 80° C. for 16 hours. The mixture was then cooled to 25° C., diluted with ethyl acetate (1.00 L), and the organic phase phase was separated, washed with water (500 mL×3), dried over sodium sulfate, filtered, and concentrated under reduced pressure. The residue was purified by silica gel chromatography (petroleum ether/ethyl acetate=1/0 to 2/1) to give 3-bromo-2-(trifluoromethyl)benzonitrile (29.0 g, 116 mmol, 72.3% yield) as a white solid.

¹H NMR (400 MHZ, DMSO-d₆) δ=8.20 (d, J=8.0 Hz, 1H), 8.10 (d, J=7.6 Hz, 1H), 7.75 (t, J=8.0 Hz, 1H).

Step B: To a solution of 3-bromo-2-(trifluoromethyl)benzonitrile (29.0 g, 116 mmol, 1.00 eq.) and tributyl(1-ethoxyvinyl) tin (50.3 g, 139 mmol, 47.0 mL, 1.20 eq.) in toluene (250 mL) was added Pd(PPh₃)₄ (6.70 g, 5.80 mmol, 0.05 eq.) under a nitrogen atmosphere, and the mixture was stirred at 100° C. for 16 hours. The reaction mixture was cooled to 25° C., diluted with water (500 mL) and ethyl acetate (200 mL), and finally followed by addition of potassium fluoride (50.0 g) solid. The mixture was stirred at 25° C. for 30 minutes, then the organic layer was separated, dried over sodium sulfate, filtered, and concentrated under reduced pressure. The residue was purified by column chromatography (SiO₂, petroleum ether/ethyl acetate=20/1 to 5/1) to afford a crude product. The crude product was triturated by petroleum ether (50.0 mL), filtered, and the filtrate was concentrated under reduced pressure to give 3-(1-ethoxyvinyl)-2-(trifluoromethyl)benzonitrile (8.00 g, 33.2 mmol, 23.0% yield) as light yellow oil.

¹H NMR (400 MHZ, CDCl₃) δ=7.82 (d, J=7.2 Hz, 1H), 7.70 (d, J=7.2 Hz, 1H), 7.65-7.59 (t, J=7.6 Hz, 1H), 4.37 (d, J=2.8 Hz, 1H), 4.25 (d, J=2.8 Hz, 1H), 3.90 (q, J=7.2 Hz, 2H), 1.36 (t, J=6.8 Hz, 3H).

Step C: To a solution of 3-(1-ethoxyvinyl)-2-(trifluoromethyl)benzonitrile (7.00 g, 29.0 mmol, 1.00 eq.) in tetrahydrofuran (10.0 mL) was added hydrochloric acid (2.00 M, 29.0 mL, 2.00 eq.), and the reaction mixture was stirred at 20° C. for 2 hours. The pH of the mixture was then adjusted to pH=8 with sodium bicarbonate aqueous solution and further diluted with water (100 mL). The resulting solution was extracted with ethyl acetate (50.0 mL×3), and the combined organic organic phases were washed with brine (100 mL), dried over sodium sulfate, filtered, and concentrated under reduced pressure. The residue was purified by column chromatography (SiO₂, petroleum ether/ethyl acetate=20/1 to 5/1) to give 3-acetyl-2-(trifluoromethyl)benzonitrile (5.30 g, 24.8 mmol, 85.6% yield) as colorless oil.

¹H NMR (400 MHZ, DMSO-d₆) δ=8.25 (dd, J=0.8, 7.6 Hz, 1H), 8.07-7.94 (m, 2H), 2.60 (s, 3H).

Step D: To a solution of 3-acetyl-2-(trifluoromethyl)benzonitrile (1.00 g, 4.69 mmol, 1.00 eq.) and (R)-2-methylpropane-2-sulfinamide (625 mg, 5.16 mmol, 1.10 eq.) in tetrahydrofuran (2.00 mL) was added 1,2-dimethoxyethane (423 mg, 4.69 mmol, 488 μL, 1.00 eq.) and titanium (IV) ethoxide (3.21 g, 14.1 mmol, 2.92 mL, 3.00 eq.), and the reaction mixture was stirred at 80° C. for 16 hours. The mixture was concentrated under reduced pressure, and the residue was diluted with ethyl acetate (100 mL) and poured into a mixture of celatom (20.0 g) and saturated sodium bicarbonate (10.0 g) in water (100 mL). The mixture was stirred then filtered, and the filter cake was stirred with ethyl acetate (30.0 mL) and filtered, the procedure was repeated three times until the cake of product was washed away. The combined filtrate was separated, and the aqueous phase was extracted with ethyl acetate (100 mL). The combined organic layers were washed with brine (50.0 mL), dried over sodium sulfate, filtered, and concentrated under reduced pressure. The residue was purified by flash silica gel chromatography (ethyl acetate/petroleum ether, 0-30%) to afford (R)—N-(1-(3-cyano-2-(trifluoromethyl)phenyl)ethylidene)-2-methylpropane-2-sulfinamide (950 mg, 2.99 mmol, 63.7% yield, 99.5% purity) as light yellow oil. LCMS [M+1]⁺: 317.1.

¹H NMR (400 MHZ, CDCl₃) δ=7.92-7.80 (m, 1H), 7.77-7.65 (m, 1H), 7.61-7.37 (m, 1H), 2.74-2.38 (m, 3H), 1.29-1.24 (m, 9H).

Step E: To a solution of (R)—N-(1-(3-cyano-2-(trifluoromethyl)phenyl)ethylidene)-2-methylpropane-2-sulfinamide (1.70 g, 5.37 mmol, 1.00 eq.) in tetrahydrofuran (20.0 mL) was added sodium borohydride (610 mg, 16.0 mmol, 3.00 eq.) portionwise under a nitrogen atmosphere at 0° C. After addition, the mixture was stirred at this temperature for 30 minutes, and then warmed to 25° C. and stirred for an additional 3 hours. The mixture was then diluted with saturated aqueous ammonium chloride (100 mL) dropwise under a nitrogen atmosphere while stirring at 25° C., then extracted with ethyl acetate (150 mL×2). The combined organic layers were dried over anhydrous sodium sulfate, filtered, and concentrated under reduced pressure.

The residue was purified by column chromatography (SiO₂, petroleum ether/ethyl acetate=5/1 to 1/1) to give (R)—N-(1-(3-cyano-2-(trifluoromethyl)phenyl)ethyl)-2-methylpropane-2-sulfinamide (1.50 g, 4.71 mmol, 87.7% yield, mixture of diastereomers) as a white solid. LCMS [M+1]⁺: 319.1.

Step F: A mixture of (R)—N-(1-(3-cyano-2-(trifluoromethyl)phenyl)ethyl)-2-methylpropane-2-sulfinamide (1.4 g, 4.40 mmol, 1.00 eq.) in HCl·dioxane (10.0 mL) was was stirred at 5° C. for 30 minutes. After this time, a white precipitate was formed and the suspension was filtered. The filter cake was collected and dried under vacuum to give 3-(1-aminoethyl)-2-(trifluoromethyl)benzonitrile (850 mg, 3.39 mmol, 77.1% yield, HCl salt) as a white solid. LCMS [M+1]⁺: 215.1.

¹H NMR (400 MHZ, DMSO-d₆) δ=8.84 (s, 3H), 8.38 (br d, J=8.0 Hz, 1H), 8.19 (d, J=7.6 Hz, 1H), 8.12-7.95 (m, 1H), 4.64 (br d, J=6.0 Hz, 1H), 1.56 (d, J=6.4 Hz, 3H).

Step G: A mixture of 3-(1-aminoethyl)-2-(trifluoromethyl)benzonitrile (300 mg, 1.40 mmol, 1.00 eq., HCl salt), 1,7-dichloro-4-methylpyrido[3,4-d]pyridazine (300 mg, 1.40 mmol, 1.00 eq.), diisopropylethylamine (499 mg, 3.86 mmol, 673 μL, 2.76 eq.) and cesium fluoride (400 mg, 2.63 mmol, 97.0 L, 1.88 eq.) in dimethylsulfoxide (1.50 mL) was degassed and purged with nitrogen (3 times), and then the mixture was stirred at 130° C. for 1 hour under a nitrogen atmosphere. The mixture was then cooled to 25° C. and ethyl acetate (60.0 mL) was added, and the organic solution was washed with brine (30.0 mL×2), dried over anhydrous sodium sulfate, filtered and concentrated under reduced pressure. The residue was purified by column chromatography (SiO₂, petroleum ether/ethyl acetate=5/1 to 1/1) to give 3-(1-((7-chloro-4-methylpyrido[3,4-d]pyridazin-1-yl)amino)ethyl)-2-(trifluoromethyl)benzonitrile (160 mg, 408 μmol, 29.2% yield) as a white solid. LCMS [M+1]⁺: 392.1.

3-(1-((7-chloro-4-methylpyrido[3,4-d]pyridazin-1-yl)amino)ethyl)-2-(trifluoromethyl)benzonitrile (160 mg) was further purified using SFC [column: DAICEL CHIRALPAK AD (250 mm×30 mm, 10 μm); mobile phase: phase A: (0.1% NH₄OH) in MeOH, phase B: CO₂; B %: 20%-20%] to give the first eluting isomer as (R)-3-(1-((7-chloro-4-methylpyrido[3,4-d]pyridazin-1-yl)amino)ethyl)-2-(trifluoromethyl)benzonitrile (62.0 mg, 158 μmol, 39.0% yield) as a white solid. LCMS [M+1]⁺: 392.1.

¹H NMR (400 MHZ, CD₃OD) δ=9.24 (d, J=0.8 Hz, 1H), 8.46 (d, J=0.8 Hz, 1H), 8.05 (d, J=8.4 Hz, 1H), 7.80 (d, J=7.2 Hz, 1H), 7.71-7.57 (m, 1H), 5.74 (q, J=6.8 Hz, 1H), 2.74 (s, 3H), 1.68 (d, J=6.8 Hz, 3H).

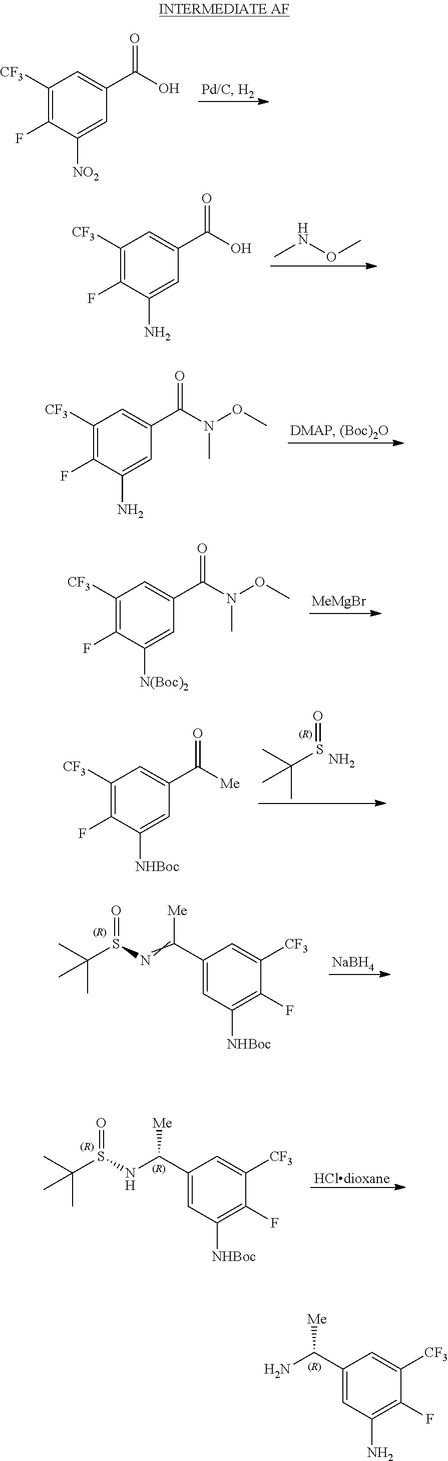

Step A: To a solution of 4-fluoro-3-nitro-5-(trifluoromethyl)benzoic acid (2.00 g, 7.90 mmol, 1.00 eq.) in tetrahydrofuran (15.0 mL) was added palladium on carbon (7.90 mmol, 10% purity, 1.00 eq.) under a nitrogen atmosphere, and the mixture was stirred at 25° C. for 2 hours under a hydrogen atmosphere (15 Psi). The mixture was then filtered and concentrated under reduced pressure to give compound 3-amino-4-fluoro-5-(trifluoromethyl)benzoic acid (1.60 g, 7.17 mmol, 90.8% yield) as a white solid.

¹H NMR (400 MHZ, DMSO-d₆) δ=7.68-7.64 (m, 1H), 7.32-7.29 (m, 1H), 5.95-5.89 (m, 2H).

Step B: To a solution of 3-amino-4-fluoro-5-(trifluoromethyl)benzoic acid (1.50 g, 6.72 mmol, 1.00 eq.) and N,O-dimethylhydroxylamine (830 mg, 13.45 mmol, 2.00 eq.) in N,N-dimethylformamide (10.0 mL) was added HATU (5.11 g, 13.5 mmol, 2.00 eq.) and N,N-diisopropylethylamine (2.61 g, 20.2 mmol, 3.50 mL, 3.00 eq.), and the mixture was stirred at 25° C. for 12 hours. The mixture was diluted with water (50.0 mL) and then extracted with ethyl acetate (50.0 mL×3). The combined organic layers were washed with brine (50.0 mL×3), dried over sodium sulfate, filtered and concentrated under reduced pressure. The residue was purified by column chromatography (silica gel, petroleum ether/ethyl acetate=10/1 to 3/1) to give compound 3-amino-4-fluoro-N-methoxy-N-methyl-5-(trifluoromethyl)benzamide (1.50 g, 5.64 mmol, 83.9% yield) as a yellow oil.

¹H NMR (400 MHZ, CDCl₃) δ=7.38-7.34 (m, 2H), 3.57 (s, 3H), 3.36 (s, 3H)

Step C: To a solution of 3-amino-4-fluoro-N-methoxy-N-methyl-5-(trifluoromethyl)benzamide (1.50 g, 5.64 mmol, 1.00 eq.) in dichloromethane (10.0 mL) was added di-tert-butyl dicarbonate (3.69 g, 16.9 mmol, 3.88 mL, 3.00 eq.) and 4-dimethylaminopyridine (688 mg, 5.64 mmol, 1.00 eq.), and the mixture was stirred at 25° C. for 12 hours. The reaction mixture was diluted with water (50.0 mL) and then extracted with ethyl acetate (50.0 mL×3). The combined organic layers were washed with brine (50.0 mL×3), dried over sodium sulfate, filtered, and concentrated under reduced pressure. The residue was purified by column chromatography (silica gel, petroleum ether/ethyl acetate=10/1 to 3/1) to give compound tert-butyl (tert-butoxycarbonyl) (2-fluoro-5-(methoxy(methyl) carbamoyl)-3-(trifluoromethyl)phenyl)carbamate (2.00 g, 4.29 mmol, 76.1% yield) as a yellow oil.

¹H NMR (400 MHZ, CDCl₃) δ=8.05-8.01 (m, 1H), 7.87-7.84 (m, 1H), 3.55 (s, 3H), 3.39 (s, 3H), 1.42 (s, 18H).

Step D: To a solution of tert-butyl (tert-butoxycarbonyl) (2-fluoro-5-(methoxy(methyl) carbamoyl)-3-(trifluoromethyl)phenyl)carbamate (1.80 g, 3.86 mmol, 1.00 eq.) in tetrahydrofuran (20.0 mL) was added methylmagnesium bromide solution (3.00 M, 3.86 mL, 3.00 eq.) at 0° C., and the mixture was stirred at 0° C. for 12 hours. The reaction mixture was then diluted with water (100 mL), and the solution was extracted with ethyl acetate (100 mL×3). The combined organic layers were washed with brine (100 mL×3), dried over sodium sulfate, filtered, and concentrated under reduced pressure. The residue was purified by column chromatography (silica gel, petroleum ether/ethyl acetate=10/1 to 3/1) to give compound tert-butyl(5-acetyl-2-fluoro-3-(trifluoromethyl)phenyl)carbamate (1.10 g, 3.42 mmol, 88.7% yield) as a yellow oil.

¹H NMR (400 MHZ, CDCl₃) δ=8.98 (d, J=6.4 Hz, 1H), 7.90-7.87 (m, 1H), 6.86 (s, 1H), 2.65 (s, 3H), 1.56 (s, 9H).

Step E: To a solution of tert-butyl(5-acetyl-2-fluoro-3-(trifluoromethyl)phenyl)carbamate (1.10 g, 2.61 mmol, 1.00 eq.) and (R)-2-methylpropane-2-sulfinamide (950 mg, 7.83 mmol, 3.00 eq.) in tetrahydrofuran (10.0 mL) were added titanium (IV) isopropoxide (1.48 g, 5.22 mmol, 1.54 mL, 2.00 eq.) and 1-methoxy-2-(2-methoxyethoxy)ethane (1.87 g, 13.97 mmol, 2.00 mL, 5.35 eq.), and the mixture was stirred at 70° C. for 12 hours. The mixture was then diluted with water (50.0 mL) and extracted with ethyl acetate (50.0 mL×3). The combined organic layers were washed with brine (50.0 mL×3), dried over sodium sulfate, filtered, and concentrated under reduced pressure. The residue was purified by column chromatography (petroleum ether/ethyl acetate=10/1 to 3/1) to give compound tert-butyl (R)-(5-(1-((tert-butylsulfinyl)imino)ethyl)-2-fluoro-3-(trifluoromethyl)phenyl)carbamate (1.00 g, 2.36 mmol, 90.1% yield) as a yellow oil.

¹H NMR (400 MHz, CDCl₃) δ=8.86 (d, J=6.4 Hz, 1H), 7.82 (d, J=6.0 Hz, 1H), 6.85 (s, 1H), 2.79 (s, 3H), 1.54 (s, 9H), 1.33 (s, 9H).

Step F: To a solution of tert-butyl (R)-(5-(1-((tert-butylsulfinyl)imino)ethyl)-2-fluoro-3-(trifluoromethyl)phenyl)carbamate (1.00 g, 2.36 mmol, 1.00 eq.) in tetrahydrofuran (10.0 mL) was added sodium borohydride (268 mg, 7.07 mmol, 3.00 eq.) at 0° C., and the mixture was stirred at 0° C. for 2 hours. The mixture was then diluted with water (50.0 mL) and extracted with ethyl acetate (50.0 mL×3). The combined organic layers were washed with brine (50.0 mL×3), dried over sodium sulfate, filtered, and concentrated under reduced pressure to give a residue. The residue was purified by column chromatography (silica gel, petroleum ether/ethyl acetate=10/1 to 3/1) to give compound tert-butyl(5-((R)-1-(((R)-tert-butylsulfinyl)amino)ethyl)-2-fluoro-3-(trifluoromethyl)phenyl)carbamate (620 mg, 1.45 mmol, 61.7% yield) as a white solid.

¹H NMR (400 MHZ, CDCl₃) δ=8.34 (d, J=6.4 Hz, 1H), 7.23-7.20 (m, 1H), 6.80 (s, 1H), 4.56-5.53 (m, 1H), 1.54-1.52 (m, 12H), 1.24 (s, 9H).

Step G: To a solution of tert-butyl(5-((R)-1-(((R)-tert-butylsulfinyl)amino)ethyl)-2-fluoro-3-(trifluoromethyl)phenyl)carbamate (620 mg, 1.45 mmol, 1.00 eq.) in dichloromethane (5.00 mL) was added hydrochloride (4.00 M in 1,4-dioxane, 5.00 mL, 13.76 eq.), and the mixture was stirred at 25° C. for 1 hour. The mixture was then concentrated under reduced pressure to give compound (R)-5-(1-aminoethyl)-2-fluoro-3-(trifluoromethyl) aniline (280 mg, 1.24 mmol, 85.5% yield, 98.6% purity, HCl salt) as a yellow oil. This compounds was used directly without further purification.

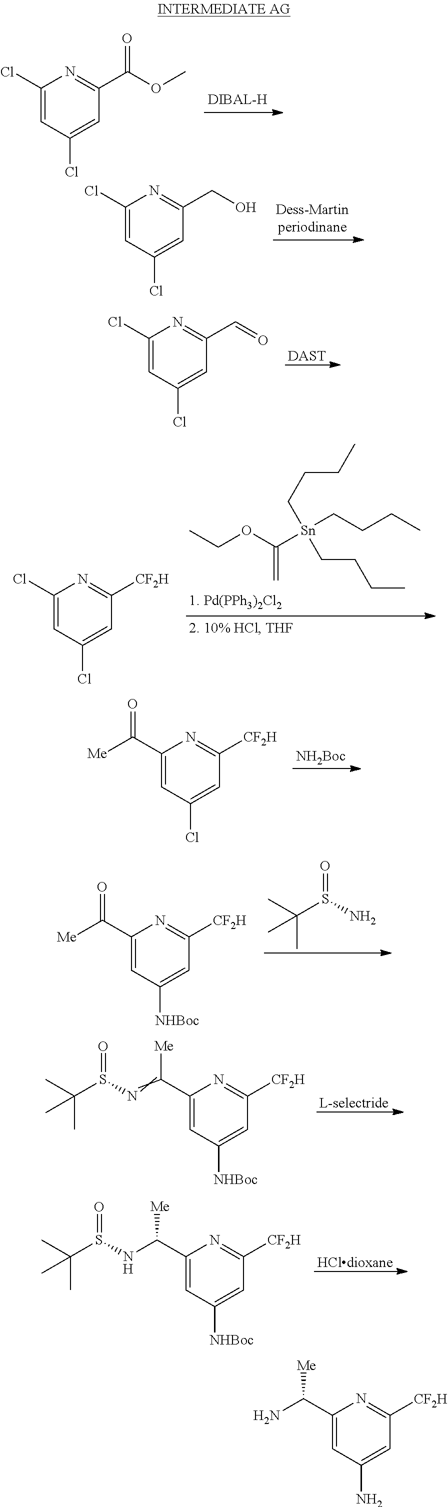

Step A: To a solution of methyl 4,6-dichloropicolinate (4.50 g, 21.8 mmol, 1.00 eq.) in dichloromethane (40.0 mL) was added DIBAL-H (1.0 M, 65.5 mL, 3.00 eq.) dropwise over 10 minutes at −78° C., and the reaction mixture was stirred at −78° C. for 2 hours. The mixture was then diluted with water (2.50 mL) dropwise at 0° C. under a nitrogen atmosphere, followed by addition of sodium hydroxide aqueous solution (2.50 mL, w/w=15%) and water (6.26 mL). The mixture was then stirred at 0° C. for 30 minutes to give a suspension, and the suspension was filtered and the filtrate was concentrated under reduced pressure. The residue was purified by column chromatography (SiO₂, petroleum ether/ethyl acetate=30/1 to 10/1) to give (4,6-dichloropyridin-2-yl) methanol (2.40 g, 13.5 mmol, 61.7% yield) as a yellow oil.

¹H NMR (400 MHZ, DMSO-d₆) δ=7.65 (s, 1H), 7.52 (s, 1H), 5.69 (t, J=6.0 Hz, 1H), 4.53 (d, J=6.0 Hz, 2H).

Step B: To a solution of (4,6-dichloropyridin-2-yl) methanol (2.40 g, 13.5 mmol, 1.00 eq.) in dichloromethane (20.0 mL) was added Dess-Martin periodinane (11.4 g, 27.0 mmol, 8.35 mL, 2.00 eq.) portionwise at 0° C., and the mixture was stirred at 20° C. for 2 hours. The mixture was then poured into water (10.0 mL) and stirred for 15 minutes, then saturated sodium thiosulfate aqueous solution (20.0 mL) was slowly added and the mixture was stirred for an additional 15 minutes. The suspension was filtered, the layers were separated, and the aqueous phase was extracted with DCM (20.0 mL×2). The combined organic layers were washed with brine (20 mL), dried over anhydrous sodium sulfate, filtered, and concentrated under reduced pressure. The residue was purified by column chromatography (SiO₂, petroleum ether/ethyl acetate=50/1 to 10/1) to give 4,6-dichloropicolinaldehyde (1.60 g, 9.09 mmol, 67.4% yield) as a red oil.

¹H NMR (400 MHz, DMSO-d₆) δ=9.87 (s, 1H), 8.14 (d, J=1.6 Hz, 1H), 8.01 (d, J=1.6 Hz, 1H).

Step C: To a solution of 4,6-dichloropicolinaldehyde (1.10 g, 6.25 mmol, 1.00 eq.) in dichloromethane (10.0 mL) was added diethylaminosulfur trifluoride (2.01 g, 12.5 mmol, 1.65 mL, 2.00 eq.) dropwise at −20° C., and the mixture was stirred at 25° C. for 1 hour. The mixture was then slowly poured into saturated sodium bicarbonate aqueous solution (10.0 mL) at 25° C., and the resulting solution was extracted with ethyl acetate (10.0 mL×3). The combined organic phases were washed with brine (5.00 mL×3), dried over anhydrous sodium sulfate, filtered, and concentrated in under reduced pressure. The residue was purified by column chromatography (SiO₂, petroleum ether/ethyl acetate=100/1 to 20/1) to give 2,4-dichloro-6-(difluoromethyl)pyridine (1.00 g, 5.05 mmol, 80.8% yield) as yellow oil.

¹H NMR (400 MHZ, CD₃OD) δ=7.75 (s, 1H), 7.74 (s, 1H), 6.82-6.55 (m, 1H).

Step D: To a solution of tributyl(1-ethoxyvinyl) tin (2.01 g, 5.56 mmol, 1.88 mL, 1.00 eq.) and 2,4-dichloro-6-(difluoromethyl)pyridine (1.10 g, 5.56 mmol, 1.00 eq.) in dioxane (10.0 mL) was added Pd(PPh₃)₂Cl₂ (390 mg, 556 μmol, 0.10 eq) under a nitrogen atmosphere, and the mixture was stirred at 110° C. for 12 hours. The reaction mixture was cooled to 25° C. and slowly poured into a saturated potassium fluoride aqueous solution (20.0 mL). The resulting aqueous solution was extracted with ethyl acetate (50.0 mL×3), and the combined organic layers were washed with brine (30.0 mL×2), dried over anhydrous sodium, filtered, and concentrated under reduced pressure. The residue was purified by column chromatography (SiO₂, petroleum ether/ethyl acetate=100/1 to 20/1) to give 4-chloro-2-(difluoromethyl)-6-(1-ethoxyvinyl)pyridine (1.20 g, 5.14 mmol, 92.5% yield) as a yellow oil which was used in the next step directly.

To a solution of 4-chloro-2-(difluoromethyl)-6-(1-ethoxyvinyl)pyridine (1.00 g, 4.28 mmol, 1.00 eq) in dioxane (5.00 mL) was added hydrochloric acid aqueous solution (2.00 M, 4.28 mL, 2.00 eq) at 20° C., and the mixture was stirred at 20° C. for 1 hour. The pH of the mixture was then adjusted to pH=8 by addition saturated sodium bicarbonate (15.0 mL), and extracted with ethyl acetate (30.0 mL×2). The combined organic phases were washed with brine (10.0 mL×2), dried over anhydrous sodium sulfate, filtered, and concentrated under reduced pressure. The residue was purified by column chromatography (SiO₂, petroleum ether/ethyl acetate=50/1 to 10/1) to give 1-(4-chloro-6-(difluoromethyl)pyridin-2-yl)ethan-1-one (800 mg, 3.89 mmol, 90.9% yield) as a white solid.

¹H NMR (400 MHZ, CD₃OD) δ=8.10-8.16 (m, 1H), 7.95 (d, J=1.6 Hz, 1H), 6.67-6.95 (m, 1H), 2.69 (s, 3H).

Step E: To a solution of 1-(4-chloro-6-(difluoromethyl)pyridin-2-yl)ethan-1-one (0.85 g, 4.13 mmol, 1.00 eq.) and tert-butyl carbamate (1.45 g, 12.4 mmol, 3.00 eq.) in dioxane (6.00 mL) was added cesium carbonate (2.69 g, 8.27 mmol, 2.00 eq.), XPhos (394 mg, 827 μmol, 0.20 eq.), and palladium acetate (92.8 mg, 413 μmol, 0.10 eq.) under a nitrogen atmosphere, and the mixture was stirred at 90° C. for 2 hours. The mixture was then cooled to 25° C. and concentrated under reduced pressure, and the residue was purified by column chromatography (SiO₂, petroleum ether/ethyl acetate=100/1 to 10/1) to give tert-butyl(2-acetyl-6-(difluoromethyl)pyridin-4-yl)carbamate (1.00 g, 3.49 mmol, 84.5% yield) as a white solid. LCMS [M+1]⁺: 287.1.

Step F: To a solution of tert-butyl(2-acetyl-6-(difluoromethyl)pyridin-4-yl)carbamate (1.00 g, 3.49 mmol, 1.00 eq.) and (S)-2-methylpropane-2-sulfinamide (508 mg, 4.19 mmol, 1.20 eq.) in THF (10.0 mL) was added titanium (IV) ethoxide (7.97 g, 34.9 mmol, 7.24 mL, 10.0 eq.), and the mixture was stirred at 75° C. for 12 hours. The mixture was then cooled to 25° C. and poured into water (5.00 mL), then the suspension was filtered, and the filtrate was concentrated under reduced pressure. The residue was purified by column chromatography (SiO₂, petroleum ether/ethyl acetate=50/1 to 5/1) to give tert-butyl (S)-(2-(1-((tert-butylsulfinyl)imino)ethyl)-6-(difluoromethyl)pyridin-4-yl)carbamate (1.00 g, 2.57 mmol, 73.5% yield) as a yellow solid.

¹H NMR (400 MHZ, CD₃OD) δ=8.30 (s, 1H), 7.94 (d, J=1.6 Hz, 1H), 6.52-6.82 (m, 1H), 2.81 (s, 3H), 1.54 (s, 9H), 1.35 (s, 9H).

Step G: To a solution of tert-butyl (S)-(2-(1-((tert-butylsulfinyl)imino)ethyl)-6-(difluoromethyl)pyridin-4-yl)carbamate (1.00 g, 2.57 mmol, 1.00 eq.) in THF (10.0 mL) was added L-selectride (1.0 M, 976 mg, 5.14 mmol, 1.12 mL, 2.00 eq.) dropwise at 0° C., and the mixture was stirred at 0-20° C. for 1 hour. The mixture was poured into saturated ammonium chloride aqueous solution (15.0 mL) and stirred for 10 minutes, then extracted with ethyl acetate (15.0 mL×3). The combined organic phases were washed with brine (15.0 mL×3), dried over anhydrous sodium sulfate, filtered, and filtrate concentrated under reduced pressure. The residue was purified by column chromatography (SiO₂, petroleum ether/ethyl acetate=100/1 to 5/1) to give tert-butyl(2-((R)-1-(((S)-tert-butylsulfinyl)amino)ethyl)-6-(difluoromethyl)pyridin-4-yl)carbamate (550 mg, 1.26 mmol, 49.0% yield, 89.5% purity) as a white solid.

¹H NMR (400 MHZ, CD₃OD) δ=7.70 (s, 1H), 7.61 (d, J=2.0 Hz, 1H), 6.41-6.77 (m, 1H), 4.55 (q, J=6.8 Hz, 1H), 1.58 (d, J=6.8 Hz, 3H), 1.53 (s, 9H), 1.23 (s, 9H).

SFC: Column: Chiralcel OD-3 50×4.6 mm I.D., 3 μm Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA Column Temp: 35° C.; Back Pressure: 100 Bar.

Step H: A solution of tert-butyl(2-((R)-1-(((S)-tert-butylsulfinyl)amino)ethyl)-6-(difluoromethyl)pyridin-4-yl)carbamate (450 mg, 1.15 mmol, 1.00 eq.) in hydrochloric acid/dioxane (2.00 mL) was stirred at 0-20° C. for 1 hour. The mixture was then concentrated under reduced pressure to give a mixture of (R)-2-(1-aminoethyl)-6-(difluoromethyl)pyridin-4-amine and tert-butyl(2-((R)-1-(((S)-tert-butylsulfinyl)amino)ethyl)-6-(difluoromethyl)pyridin-4-yl)carbamate as a white solid which was used directly in the next step directly without purification. LCMS [M+1]⁺: 288.2.

¹H NMR (400 MHZ, CD₃OD) δ=7.74 (s, 1H), 7.65 (d, J=1.6 Hz, 1H), 6.82-6.51 (m, 1H), 4.60-4.45 (m, 2H), 1.61 (d, J=6.8 Hz, 3H), 1.54 (s, 9H).

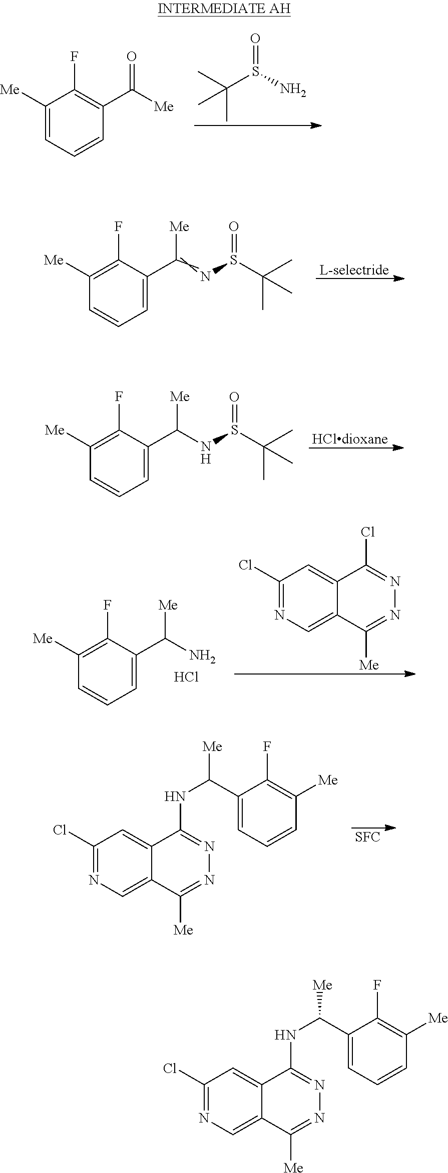

Step A: To a solution of 1-(2-fluoro-3-methylphenyl)ethan-1-one (1.00 g, 6.57 mmol, 1.00 eq.) and (S)-2-methylpropane-2-sulfinamide (1.04 g, 8.54 mmol, 1.30 eq.) in tetrahydrofuran (20.0 mL) were added titanium tetrisopropyloxide (3.73 g, 13.1 mmol, 3.88 mL, 2.00 eq.) under a nitrogen atmosphere, and the mixture was stirred at 70° C. for 12 hours under a nitrogen atmosphere. The reaction mixture was cooled to 25° C. and poured into water (40.0 mL) to give a suspension after stirring for 10 minutes, the suspension was filtered, the resulting aqueous solution was extracted with ethyl acetate (40.0 mL×3). The combined organic layers were washed with brine (30.0 mL), dried over sodium sulfate, filtered, and concentrated under reduced pressure. The residue was purified by column chromatography (silica gel, petroleum ether/ethyl acetate=50/1 to 2/1) to give (S)—N-(1-(2-fluoro-3-methylphenyl)ethylidene)-2-methylpropane-2-sulfinamide (1.50 g, 5.87 mmol, 89.4% yield) as a yellow solid. LCMS [M+1]⁻: 256.2.

¹H NMR (400 MHZ, DMSO-d₆) δ=7.46 (br t, J=6.8 Hz, 1H), 7.30-7.24 (m, 1H), 7.09-7.04 (m, 1H), 2.76 (br d, J=2.8 Hz, 3H), 2.31 (d, J=2.4 Hz, 3H), 1.31 (s, 9H).

Step B: To a solution of (S)—N-(1-(2-fluoro-3-methylphenyl)ethylidene)-2-methylpropane-2-sulfinamide (1.50 g, 5.87 mmol, 1.00 eq.) in tetrahydrofuran (20.0 mL) was added L-selectride (1.0 M, 11.7 mmol, 11.8 mL, 2.00 eq.) at −78° C. under a nitrogen atmosphere, and the mixture was stirred at −78° C. for 2 hours. The reaction mixture was poured into water (10.0 mL) slowly and stirred for 10 minutes, and the resulting mixed solution was extracted with ethyl acetate (10.0 mL×3). The combined organic layers were washed with brine (10.0 mL), dried over sodium sulfate, filtered, and concentrated under reduced pressure. The residue was purified by column chromatography (silica gel, petroleum ether/ethyl acetate=50/1 to 1/1) to give (S)—N-(1-(2-fluoro-3-methylphenyl)ethyl)-2-methylpropane-2-sulfinamide (900 mg, 3.50 mmol, 59.5% yield) as a yellow oil. LCMS [M+1]⁺: 258.4.

¹H NMR (400 MHZ, DMSO-d₆) δ=7.16 (t, J=7.6 Hz, 1H), 7.13-7.08 (m, 1H), 7.04-6.99 (m, 1H), 4.85 (q, J=6.8 Hz, 1H), 2.28 (d, J=2.0 Hz, 3H), 1.58 (d, J=6.8 Hz, 3H), 1.20 (s, 9H).

Step C: To a solution of (S)—N-(1-(2-fluoro-3-methylphenyl)ethyl)-2-methylpropane-2-sulfinamide (900 mg, 3.50 mmol, 1.00 eq.) in dichloromethane (5.00 mL) was added HCl (4.00 M in 1,4-dioxane, 5.00 mL, 5.72 eq.) under nitrogen a atmosphere, and the mixture was stirred at 20° C. for 1 hour. The mixture was concentrated to give 1-(2-fluoro-3-methylphenyl)ethan-1-amine (390 mg, crude, hydrochloride salt) as a yellow solid which was used directly without further purification.

To a solution of 1-(2-fluoro-3-methylphenyl)ethan-1-amine (300 mg, 1.96 mmol, 1.00 eq., hydrochloride salt), 1,7-dichloro-4-methylpyrido[3,4-d]pyridazine (419 mg, 1.96 mmol, 1.00 eq.), N,N-diisopropylethylamine (506 mg, 3.92 mmol, 2.00 eq.) and potassium fluoride (341 mg, 5.87 mmol, 0.14 mL, 3.00 eq.) in dimethyl sulfoxide (5.00 mL) were stirred at 130° C. for 1 hour under a nitrogen atmosphere. The mixture was then cooled to 25° C., poured into water (20.0 mL), and extracted with ethyl acetate (20.0 mL×3). The combined organic layers were washed with brine (20.0 mL), dried over sodium sulfate, filtered, and concentrated under reduced pressure. The residue was purified by prep-HPLC [column: Welch Xtimate C18 150×25 mm×5 um; mobile phase: phase A: water (0.05% HCl), phase B: acetonitrile; B %: 14%-44%] to give 7-chloro-N-(1-(2-fluoro-3-methylphenyl)ethyl)-4-methylpyrido[3,4-d]pyridazin-1-amine (100 mg, 0.30 mmol, 23.2% yield) as a yellow solid. LCMS [M+1]⁺: 331.2.

A racemic 7-chloro-N-(1-(2-fluoro-3-methylphenyl)ethyl)-4-methylpyrido[3,4-d]pyridazin-1-amine (200 mg, 0.60 mmol, 1.00 eq.) was purified by SFC (column: DAICEL CHIRALPAK IG (250 mm×30 mm, 10 um); mobile phase: phase A: 0.1% NH₄OH in MeOH, phase B: CO₂; B %: 30%-30%] to give (R)-7-chloro-N-(1-(2-fluoro-3-methylphenyl)ethyl)-4-methylpyrido[3,4-d]pyridazin-1-amine as the first eluting isomer (80.0 mg, 0.24 mmol, 40.0% yield) as a yellow solid.

The following Examples are intended to illustrate further certain embodiments of the invention and are not intended to limit the scope of the invention.

### Example 12-1

### (R)-4-methyl-7-(4-(1-methyl-1H-pyrazol-4-yl) piperazin-1-yl)-N-(1-(2-methyl-3-(trifluoromethyl)phenyl)ethyl)pyrido[3,4-d]pyridazin-1-amine

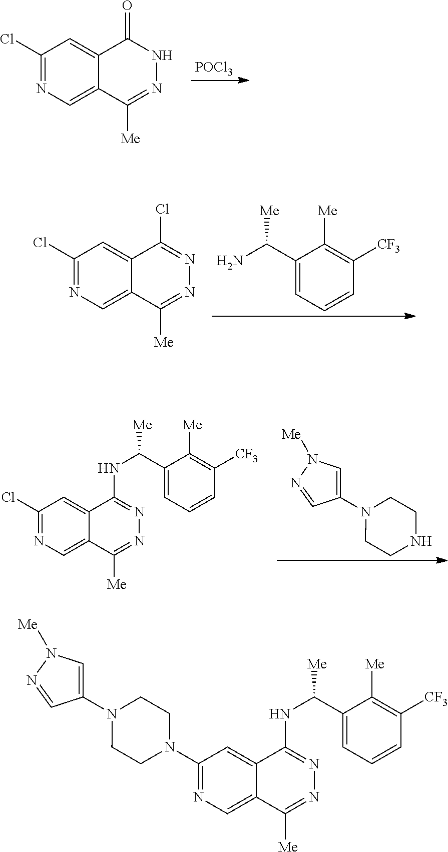

Step A: A solution of 7-chloro-4-methylpyrido[3,4-d]pyridazin-1(2H)-one (5.00 g, 25.6 mmol, 1.00 eq.) in POCl₃ (137 g, 893 mmol, 83.0 mL, 34.9 eq.) was added N,N-diisopropylethylamine (9.91 g, 76.7 mmol, 13.4 mL, 3 eq.) dropwise at 25° C., then the reaction was stirred at 110° C. for 2 h. After this time the mixture was cooled to 25° C. and concentrated under vacuum to give a residue, the residue was diluted with ethyl acetate (300 mL) at 0° C., adjusted to pH=7 with slow addition of sodium bicarbonate saturated aqueous solution. The combined organic phases were washed with brine (200 mL×2), dried over anhydrous sodium sulfate, filtered, and concentrated under vacuum to give 1,7-dichloro-4-methylpyrido[3,4-d]pyridazine (4.10 g, 19.2 mmol, 74.9% yield) as a pink solid.

¹H NMR (400 MHZ, DMSO-d₆) δ=9.65 (s, 1H), 8.22 (s, 1H), 3.02 (s, 3H).

Step B: To a solution of 1,7-dichloro-4-methylpyrido[3,4-d]pyridazine (300 mg, 1.40 mmol, 1.00 eq.) and (R)-1-(2-methyl-3-(trifluoromethyl)phenyl)ethan-1-amine (285 mg, 1.40 mmol, 1.00 eq.) in DMSO (5.00 mL) was added potassium fluoride (244 mg, 4.20 mmol, 98.5 μL, 3.00 eq.) and N,N-diisopropylethylamine (543 mg, 4.20 mmol, 732 μL, 3.00 eq.). The mixture was stirred at 130° C. for 12 hours, then cooled to room temperature and water (20.0 mL) was added. The mixture was extracted with ethyl acetate (10.0 mL×3), and the combined organic layers were washed with brine (5.00 mL×2), dried over sodium sulfate, filtered, and concentrated under reduced pressure to give a residue. The residue was purified by column chromatography (SiO₂, petroleum ether/ethyl acetate=1/0 to 5/1) to give (R)-7-chloro-4-methyl-N-(1-(2-methyl-3-(trifluoromethyl)phenyl)ethyl)pyrido[3,4-d]pyridazin-1-amine (320 mg, 840 μmol, 60.0% yield) as a yellow solid. LCMS [M+1]⁺: 381.0.

Step C: A mixture of (R)-7-chloro-4-methyl-N-(1-(2-methyl-3-(trifluoromethyl)phenyl)ethyl)pyrido[3,4-d]pyridazin-1-amine (40.0 mg, 105 μmol, 1.00 eq.), 1-(1-methyl-1H-pyrazol-4-yl) piperazine (58.9 mg, 210 μmol, 2.00 eq., TFA salt), cesium carbonate (171 mg, 525 μmol, 5.00 eq.), RuPhos Pd G3 (8.79 mg, 10.5 μmol, 0.10 eq.) in dioxane (1.00 mL) was degassed and purged with nitrogen 3 times, and then the mixture was stirred at 80° C. for 10 hours under a nitrogen atmosphere. The reaction mixture was quenched by addition water (15.0 mL) at 20° C., and then extracted with ethyl acetate (5.00 mL×3). The combined organic layers were washed with brine (5.00 mL), dried over sodium sulfate, filtered, and concentrated under reduced pressure to give a residue. The residue was purified by prep-HPLC (column: Phenomenex Gemini-NX C18 75×30 mm×3 μm; mobile phase: phase A: water (0.04% HCl), phase B: acetonitrile; gradient: B %: 30%-60%) to give (R)-4-methyl-7-(4-(1-methyl-1H-pyrazol-4-yl) piperazin-1-yl)-N-(1-(2-methyl-3-(trifluoromethyl)phenyl)ethyl)pyrido[3,4-d]pyridazin-1-amine (9.51 mg, 15.1% yield, HCl salt) as a light yellow solid. LCMS [M+1]⁺: 511.1.

¹H NMR (400 MHZ, DMSO-d₆) δ=9.00 (s, 1H), 7.72 (d, J=7.6 Hz, 1H), 7.57 (d, J=6.4 Hz, 1H), 7.52 (d, J=7.6 Hz, 1H), 7.48 (s, 1H), 7.36 (s, 1H), 7.35-7.29 (m, 1H), 7.25 (s, 1H), 5.63 (quin, J=6.8 Hz, 1H), 3.88-3.83 (m, 4H), 3.75 (s, 2H), 3.78-3.72 (m, 1H), 3.04-2.98 (m, 4H), 2.56 (s, 6H), 1.55 (d, J=6.8 Hz, 3H).

### Example 12-2

### 7-(6-oxa-3-azabicyclo[3.1.1]heptan-3-yl)-4-methyl-N—((R)-1-(2-methyl-3-(trifluoromethyl)phenyl)ethyl)pyrido[3,4-d]pyridazin-1-amine

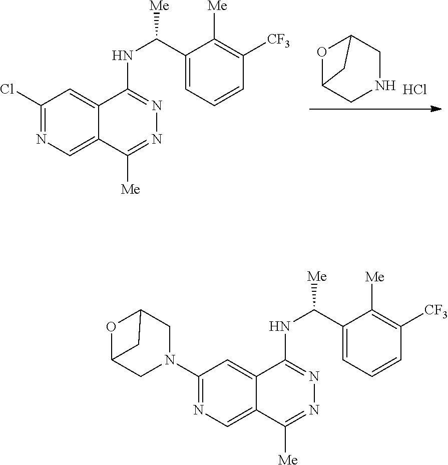 [SMILES: Cc1c([C@@H](C)Nc2nnc(C)c3cnc(Cl)cc23)cccc1C(F)(F)F ; C1NCC2CC1O2 ; Cl ; Cc1c([C@@H](C)Nc2nnc(C)c3cnc(N4CC5CC(C4)O5)cc23)cccc1C(F)(F)F]

To a solution of (R)-7-chloro-4-methyl-N-(1-(2-methyl-3-(trifluoromethyl)phenyl)ethyl)pyrido[3,4-d]pyridazin-1-amine (50.0 mg, 131 μmol, 1.00 eq.) and 6-oxa-3-azabicyclo[3.1.1]heptane (35.6 mg, 263 μmol, 2.00 eq., HCl) in dioxane (2.00 mL) was added cesium carbonate (171 mg, 525 μmol, 4.00 eq.), RuPhos (6.10 mg, 13.1 μmol, 0.10 eq.) and Pd₂(dba)₃ (6.00 mg, 6.60 μmol, 0.05 eq.) under a nitrogen atmosphere. The mixture was stirred at 110° C. for 2 hours then cooled to 25° C., filtered, and the filtrate was quenched with water (10.0 mL), and then extracted with ethyl acetate (30.0 mL). The combined organic layers were washed with brine (10.0 mL), dried over anhydrous sodium sulfate, filtered, and concentrated under reduced pressure to give a residue. The residue was purified by prep-HPLC (column: Waters Xbridge BEH C18 100×25 mm×5 μm; mobile phase: phase A: water (10 mM NH₄HCO₃), phase B: acetonitrile; gradient: B %: 30%-60%) to give 7-(6-oxa-3-azabicyclo[3.1.1]heptan-3-yl)-4-methyl-N—((R)-1-(2-methyl-3-(trifluoromethyl)phenyl)ethyl)pyrido[3,4-d]pyridazin-1-amine (8.64 mg, 18.9 μmol, 14.4% yield) as a yellow solid. LCMS [M+1]⁺: 444.1.

¹H NMR (400 MHZ, DMSO-d₆) δ=9.03 (s, 1H), 7.74 (d, J=7.6 Hz, 1H), 7.57 (s, 1H), 7.52 (d, J=8.0 Hz, 1H), 7.31 (t, J=8.0 Hz, 1H), 7.27 (s, 1H), 5.66 (t, J=7.2 Hz, 1H), 4.80 (d, J=6.4 Hz, 2H), 3.91-3.90 (m, 2H), 3.75-3.68 (m, 2H), 3.23-3.16 (m, 1H), 2.57 (s, 6H), 1.95 (d, J=9.2 Hz, 1H), 1.55 (d, J=7.2 Hz, 3H).

SFC conditions: Chiralcel OD-3 3 μm, 0.46 cm id×5 cm L; Mobile phase: A for SFC CO2 and B for MeOH (0.05% isopropylamine); Gradient: B in A from 10% to 40% in 3 minutes; Flow rate: 4.0 mL/min; Column temperature: 35° C.; Wavelength: 220 nm; System Back Pressure: 100 bar.

Following the teachings of the General Reaction Scheme III, and the procedure described for the preparation of Examples 12-1 and 12-2, the following compound of Example 12-10 shown in Table 1 was prepared.

TABLE 1

| Ex. # | Structure | Spectral Data |
|---|---|---|
| 12-10 | 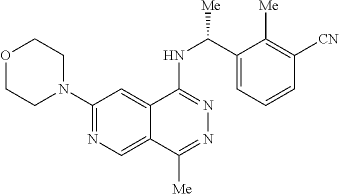 [SMILES: Cc1c(C#N)cccc1[C@@H](C)Nc1nnc(C)c2cnc(N3CCOCC3)cc12] | ¹H NMR (400 MHz, DMSO- d₆) δ = 9.00 (s, 1H), 7.71 (d, J = 8.0 Hz, 1H), 7.61 (d, J = 1.2 7.6, Hz, 1H), 7.53 (d, J = 6.8 Hz, 1H), 7.40 (s, 1H), 7.32 (t, J = 7.6 Hz, 1H), 5.53 (m, 1H), 3.74-3.83 (m, 4H), 3.63-3.73 (m, 4H), 2.66 (s, 3H), 2.56 (s, 3H), 1.54 (d, J = 6.8 Hz, 3H). LCMS [M + 1]⁺: 389.1 |
| | (R)-2-methyl-3-(1-((4-methyl-7- | |
| | morpholinopyrido[3,4-d]pyridazin-1- | |
| | yl)amino)ethyl)benzonitrile | |

The fumaric salt of MRTX-0902 was prepared by reacting MRTX-0902 with fumaric acid in the presence of a solvent.

The maleate salt of MRTX-0902 can be prepared using similar techniques, e.g., by reacting MRTX-0902 with maleic acid in the presence of a solvent.

### Example A

This Example illustrates that exemplary compounds of the present invention bind to SOS1 and prevent a labeled tracer ligand from occupying the SOS1 binding site.

The ability of a provided compounds to bind to SOS1 was measured using a HTRF displacement assay. A recombinant human SOS1 polypeptide (corresponding to amino acids 560-1049, expressed in E. Coli with N-terminal His-TEV-AviTag-SOS1 (MW=59.4 kDa) and lanthanide labeled streptavidin (CisBio) was incubated with an exemplary provided compounds (in a DMSO stock solution) in buffer (25 mM HEPES pH 7.5, 25 mM NaCl, 1 mM DTT, 0.01% Brij 35, 0.02% BSA, 0.1% DMSO). After a 10-15 minute incubation at room temperature, a solution comprised of a custom-made Cy5 labelled tracer and MAb Anti-6HIS Tb cryptate Gold (Cisbio 61H12TLA) in buffer was added to the solution containing the SOS1 polypeptide and exemplary provided compounds. After a 1-hour incubation at room temperature, the HTRF signal was measured using Clairostar plate reader (BMG Labtech) according to the manufacturer's instructions. Excitation filter EX-TR was used, and emission 1 was detected at 650-610 nm and emission 2 detected at 620-610 nm. The HTRF ratio was calculated using the formula: [emission 1/emission 2]*10000.

Background signals were calculated from well with a 10 μM inhibitor, known to inhibit 100% at that concentration. The background subtracted signals were converted to % binding relative to DMSO controls. Data were analyzed using XLFIT software (IDBS) using a Morrison equation for competitive binding and Ki's were generated for provided compounds.

For Example 12-10, Kᵢ (nM) was 1.78.

Thus, exemplary compounds of the present invention potently inhibited the binding of a SOS1 labeled tracer to SOS1 protein.

### Example B

This Example illustrates that exemplary compounds of the present invention prevent KRas-mediated GTP nucleotide exchange mediated by SOS1 to inhibit KRas activity thereby inhibiting the generation of the downstream effector pERK.

MKN1 cells (15,000/w) or H358 (30,000/w) were seeded in a black clear flat bottom 96-well cell culture plate (Corning, #3904) and incubated at 37° C. overnight. Assay day 1, cells were dosed with the compounds of the invention with a 10 μm starting concentration and serially diluted 3× for a total of 9 concentrations. The cells were incubated for approximately 0.5-1 hour with the compounds solubilized in DMSO at 37° C. Cells were immediately fixed by adding 50 μL of 4% formaldehyde to all wells in a fume hood and the plates were incubated for 20 minutes at room temperature. The formaldehyde was discarded from the plates and 150 μL of ice-cold methanol was added to permeabilize the cells for 10 minutes at −20° C. The methanol was discarded from each of the plates and any liquid remaining in the plate by tapping the plate against paper towels. Cells were then blocked with 150 μL of Odyssey blocking buffer (LI-COR Biosciences #927-50010) using 0.05% Tween for 1 hour at room temperature on a shaker. The blocking buffer was discarded and 50 μL of primary antibodies pERK (cell signaling Technology #9101L; Rabbit, 1:500) and GapDH (Millipore #MAB34; Mouse, 1:5000) diluted in Odyssey blocking buffer was added. The plates were incubated overnight at 4° C. on a shaker.

On Assay day 2, the primary antibody solution was removed. Each plate was washed 3× times with 150 μL of 1×PBST (PBS+0.1% Tween 20) and incubated with 50 μL of secondary antibodies: Anti-Rabbit (LI-COR Biosciences #926-32211) and Anti-Mouse (LI-COR Biosciences #68070) at 1:800 dilution in Odyssey blocking buffer with Tween at room temperature on a shaker for 2 hours (protected from light). The secondary antibody solution as removed and each plate was washed with PBST 3× times. Any liquid remaining was discarded and the plate was imaged using the Licor Odyssey machine according to the manufacturer's instruction, using a set focus length at 3 mm and both 800 nm and 700 nm filters. The GAPDH normalized scan values for each well were divided by the average of vehicle wells to get the % of pERK inhibition. The IC₅₀ values were then calculated with the Graph pad Prism software.

The IC₅₀ (nM) for Example 12-10 was 46.

This result illustrates that the compounds of the present invention are capable of potently inhibiting KRas-mediated activation and formation of pERK thereby blocking intracellular KRas-mediated signaling.

### Example C

### Preparation of a Fumarate Salt of MRTX0902

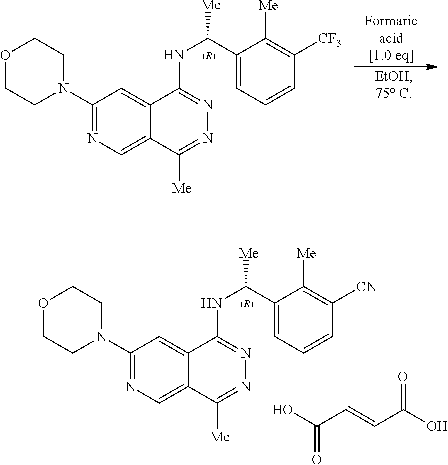

To a 500 L reactor was added (R)-2-methyl-3-(1-((4-methyl-7-morpholinopyrido[3,4-d]pyridazin-1-yl)amino)ethyl)benzonitrile [7.85 kg, 20.2 mol, 1.0 equiv.] and EtOH [86.4 L]. The suspension was heated to 75° C. Crystalline seed material [39.5 g] of the final product was added. Then a prepared solution of fumaric acid [2.40 kg, 20.2 mol, 1.0 equiv.] in 95% aqueous ethanol [52.2 L] was added dropwise. Crystallization was observed upon addition of the fumaric acid solution and stirring was continued at 75° C. for 2 h after the end of the addition. The reaction mixture was cooled to 20° C. over 4 h and then stirring was continued for 4 h at the same temperature. The suspension was filtered and the collected solid was rinsed with EtOH [23.6 L]. The wet cake was then dried at 50° C. under vacuum to afford 8.93 kg of (R)-2-methyl-3-(1-((4-methyl-7-morpholinopyrido[3,4-d]pyridazin-1-yl)amino)ethyl)benzonitrile fumarate in 97% yield.

M.p.: 253.2-253.3° C.

¹H NMR (400 MHz, DMSO-d₆) δ 9.01 (s, 1H), 7.71 (dd, J=7.9, 1.4 Hz, 1H), 7.64-7.55 (m, 2H), 7.42 (s, 1H), 7.32 (t, J=7.8 Hz, 1H), 6.61 (s, 2H), 5.51 (s, 1H), 3.80-3.74 (m, 4H), 3.73-3.66 (m, 4H), 2.65 (s, 3H), 2.56 (s, 3H), 1.54 (d, J=7.0 Hz, 3H).

¹³C NMR (101 MHZ, DMSO-d₆) δ 16.7, 17.7, 21.4, 45.0, 46.7, 65.8, 93.2, 112.3, 113.8, 118.5, 124.9, 127.0, 129.2, 130.8, 134.1, 138.7, 145.8, 147.2, 149.3, 150.8, 159.5, 166.2.

HRMS (ESI) calculated for C₂₂H₂₅N₆O: 389.2085 [M+H]⁺, Found: 389.2085.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A compound selected from the group consisting of:

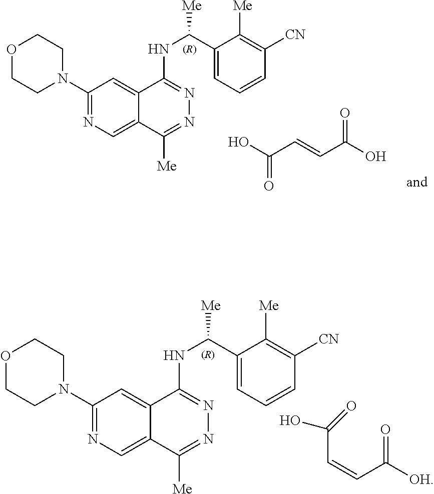

2. The compound of claim 1, wherein the compound is

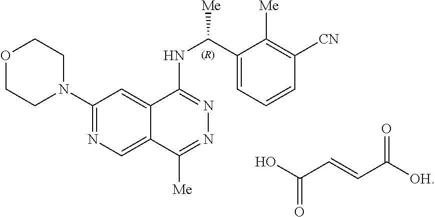

3. The compound of claim 1, wherein the compound is

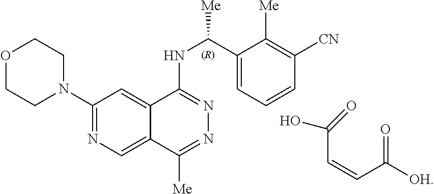

4. A pharmaceutical composition, comprising a therapeutically effective amount of the compound according to claim 1 or a pharmaceutically acceptable salt or solvate thereof, and a pharmaceutically acceptable excipient.

5. A method for inhibiting SOS1 activity in a cell, comprising contacting the cell in which inhibition of SOS1 activity is desired with an effective amount of the compound according to claim 1 or a pharmaceutically acceptable salt or solvate thereof, or a pharmaceutical composition according to claim 4.

6. The method according to claim 5, wherein the cell harbors an activating mutation in a RAS family-member gene.

7. The method according to claim 5, wherein the cell harbors an activating mutation in SOS1 gene.

8. The method according to claim 5, wherein the cell harbors an activating mutation in NF-1 or NF-2 gene.

9. A method for treating cancer comprising administering to a patient having cancer a therapeutically effective amount of the compounds according to claim 1 or a pharmaceutically acceptable salt or solvate thereof, or a pharmaceutically acceptable salt or solvate thereof, alone or combined with a pharmaceutically acceptable carrier, excipient or diluents.

10. The method according to claim 9, wherein the therapeutically effective amount of the compound is between about 0.01 to 300 mg/kg per day.

11. The method according to claim 10, wherein the therapeutically effective amount of the compound is between about 0.1 to 100 mg/kg per day.

12. The method according to claim 9, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial wcarcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

13. The method according to claim 9, wherein the cancer is a Ras family-associated cancer.

14. The method according to claim 13, wherein the Ras family-associated cancer is a KRas, HRas or NRas G12C-associated cancer, a KRas, HRas or NRas G12D-associated cancer, a KRas, HRas or NRas G12S-associated cancer, a KRas, HRas or NRas G12A-associated cancer, a KRas, HRas or NRas G13D-associated cancer, a KRas, HRas or NRas G13C-associated cancer, a KRas, HRas or NRas Q61X-associated cancer, a KRas, HRas or NRas A146T-associated cancer, a KRas, HRas or NRas A146V-associated cancer or a KRas, HRas or NRas A146P-associated cancer.

15. The method according to claim 14, wherein the Ras family-associated cancer is a KRas G12C-associated cancer.

16. The method according to claim 15, wherein the Ras family-associated cancer is non-small cell lung cancer or pancreatic cancer.

17. The method according to claim 9, wherein the cancer is a SOS1-associated cancer.

18. The method according to claim 17, wherein the SOS1-associated cancer is a SOS1 N233S-associated cancer or a SOS1 N233Y-associated cancer.

19. The method according to claim 17, wherein the SOS1-associated cancer is lung adenocarcinoma, embryonal rhabdomyosarcoma, Sertoli cell testis tumor or granular cell tumors of the skin.

20. The method according to claim 9, wherein the cancer is a NF-1/NF-2-associated cancer.
